(19) United States
(12) Patent Application Publication
(10) Pub. No.: US 2005/0070607 A1
(43) Pub. Date: Mar. 31, 2005

(54) N-acetylcysteine compositions and methods for the treatment and prevention of cysteine/glutathione deficiency in diseases and conditions

(76) Inventors: James Andrus, Redwood City, CA (US); Leonard Herzenberg, Stanford, CA (US); Leonore Herzenberg, Stanford, CA (US); David Aiello, Woodbridge (CA); John J. Mantovani, Menlo Park, CA (US)

Correspondence Address: GREENBERG TRAURIG, LLP, 885 Third Avenue, New York, NY, 10022, US

(21) Appl. No.: 10/921,679

(22) Filed: Aug. 19, 2004

Related U.S. Application Data
(60) Provisional application No. 60/496,119, filed on Aug. 19, 2003.
(60) Provisional application No. 60/496,127, filed on Aug. 19, 2003.

(51) Int. Cl. A61K 31/198

(52) U.S. Cl. 514/562

(57) ABSTRACT

Life-threatening hepatotoxicity in the setting of acetaminophen overdose is due to depletion of glutathione (GSH), a vital cysteine-containing tripeptide that protects cells and organs against oxidant injury. Rapid administration of N-acetylcysteine (NAC), which provides the cysteine necessary to replenish the depleted GSH, is the standard of care for preventing injury in acetaminophen overdose. Beneficial effects of NAC treatment have also been demonstrated in respiratory, cardiovascular, endocrine and infectious and other diseases. In fact, over fifty randomized placebo-controlled trials conducted in diverse clinical settings document positive responses to NAC treatment. The present invention relates to cysteine/glutathione (GSH) deficiency as a previously unrecognized clinical entity that can complicate the course of commonly encountered diseases and methods of treatment of this generalized deficiency involving administering N-acetylcysteine (NAC) or a pharmaceutically acceptable salt or derivative to a subject in need thereof and monitoring the subjects appropriate glutathione blood levels as needed.

This application claims the benefit of the filing date of provisional applications Ser. No. 60/496,119 and 60/496,127, both of which were filed Aug. 19, 2003.

### GOVERNMENT SUPPORT

This work is supported at least in part by grants from the N.I.H. CA42509-14. The government may have certain rights in this invention.

### FIELD OF THE INVENTION

The present invention relates to cysteine/glutathione deficiency as a previously unrecognized clinical entity that can complicate the course of commonly encountered diseases and methods of teatment of this generalized deficiency comprising administering N-acetylcysteine or a pharmaceutically acceptable salt or derivative to patients in need thereof and monitoring the appropriate glutathone blood levels as needed.

### SUMMARY OF THE INVENTION

The present invention relates to cysteine/glutathione (GSH) deficiency as a previously unrecognized clinical entity that can complicate the course of commonly encountered diseases and methods of treatment of this generalized deficiency comprising administering N-acetylcysteine (NAC) or a pharmaceutically acceptable salt or derivative and monitoring the appropriate glutathione blood levels as needed.

According to one embodiment of the invention, a method of treatment to prevent development of gluathione deficiency as a consequence of disease, a treatment, or a condition comprises administering to a subject at risk of glutathione deficieny as a consequence of disease, a treatment, or a condition a therapeutic amount of NAC or a pharmaceutically acceptable salt or derivative suficient to increase intracellular glutathione levels or decrease oxidative stress and measuring and monitoring the level of glutathione in the blood of patients as needed. According to another embodiment of the invention, a method of treatment to restore glutathione levels comprises administering to subjects in need of glutathone level restoration, as determined by measurement or by a physician, a therapeutic amount of NAC or a pharmaceutically acceptable salt or derivative thereof sufficient to increase intracellular glutathione levels or decrease oxidative stress and monitoring restoration by measuring the level of glutathione in blood as needed. According to another embodiment of the invention, a method of treatment to decrease oxidized glutathione levels elevated as a consequence of disease, a treatment, or a condition comprises administering to a subject suffering from oxidative stress a therapeutic amount of NAC or a pharmaceutically acceptable salt or derivative sufficient to decrease oxidized glutathione levels elevated as a consequence of disease and monitoring the level of oxidized glutathione in blood as needed.

### BACKGROUND OF THE INVENTION

GSH is a central component of the oxidative-reductive (redox) apparatus of every cell. One of its key functions is to combine with, and thereby inactivate (detoxify), reactive oxidative intermediates (ROI), other oxidative molecules, and certain drugs, exogenous chemicals and toxins. Because GSH is depleted in these reactions, it must continually be replenished to maintain cell and organ viability and to support normal cellular functions. Drug intoxications resulting in severe GSH depletion, notably acetaminophen overdose, cause extensive hepatic injury if treatment to replenish GSH is not initiated before GSH stores are depleted to below critical protective levels.

Synthesis of GSH requires cysteine, a conditionally essential amino acid that must be obtained from dietary sources or by conversion of dietary methionine via the cystathionase pathway. If the supply of cysteine is adequate, normal GSH levels are maintained. In contrast, if supplies of cysteine are inadequate to maintain GSH homeostasis in the face of increased GSH consumption, GSH depletion occurs.

GSH depletion impacts a wide variety of cellular processes, ranging from DNA synthesis and gene expression to sugar metabolism and lactate production. The pleiotropic activity of this key intracellular molecule, which arose very early in evolution, derives from its participation in the energy economy and the synthetic and catabolic activities of virtually all cells. In higher animals, it also participates in regulating the expression or activity of extracellular molecules, including many of the cytokines and adhesion molecules implicated in inflammatory reactions and other disease processes.

Acute GSH depletion causes severe—often fatal—oxidative and/or alkylation injury. This injury can be prevented (in acetaminophen overdose, for example) by rapid treatment with N-acetylcysteine (NAC), an efficient non-toxic source of cysteine, which is necessary to replenish hepatocellular GSH. Chronic or slowly arising GSH deficiency due to administration of GSH-depleting drugs, or to diseases and conditions that deplete GSH, can be similarly debilitating.

Systematic review of a series of randomized placebo-controlled trials conducted over the last 25 years demonstrates that treatment with NAC provides beneficial effects in a number of respiratory, cardiovascular, endocrine and infectious and other disease settings. Rapid administration of NAC is the standard of care for preventing hepatic injury in acetaminophen overdose. It provides the cysteine necessary to replenish a crucial intracellular tripeptide \tilde{□} glutamylcysteinylglycine, commonly known as GSH, which is depleted during detoxification of excessive amounts of acetaminophen. Since orally-administered NAC is rapidly converted by first-pass metabolism to cysteine, it results in replenishment of GSH as well as supplying cysteine for additional metabolic and protein synthetic processes. Thus, the beneficial effects observed following NAC treatment in the trials reviewed here suggest that cysteine/GSH deficiency contributes to the pathophysiology of a wide range of diseases and that avoidance of this deficiency may be important in treating these diseases.

GSH is a central component of the oxidative-reductive (redox) apparatus of every cell. One of its key functions is to combine with, and thereby inactivate (detoxify), reactive oxygen intermediates (ROI), other oxidative molecules, and certain drugs, exogenous chemicals and toxins. Because GSH is depleted in these reactions, it must continually be replenished to maintain cell and organ viability and to support normal cellular functions. Drug intoxications resulting in severe GSH depletion, notably acetaminophen overdose, cause extensive hepatic injury if treatment to replenish GSH is not initiated before GSH stores are depleted to below critical protective levels.

Synthesis of GSH requires cysteine, a conditionally essential amino acid that must be obtained from dietary sources or by conversion of dietary methionine via the cystathionase pathway. If the supply of cysteine is adequate, normal GSH levels are maintained. In contrast, if supplies of cysteine are inadequate to maintain GSH homeostasis in the face of increased GSH consumption, GSH depletion occurs.

GSH depletion impacts a wide variety of cellular processes, ranging from DNA synthesis and gene expression to sugar metabolism and lactate production. The pleiotropic activity of this key intracellular molecule, which arose very early in evolution, derives from its participation in the energy economy and the synthetic and catabolic activities of virtually all cells. In higher animals, it also participates in regulating the expression or activity of extracellular molecules, including many of the cytokines and adhesion molecules implicated in inflammatory reactions and other disease processes.

Acute GSH depletion causes severe—often fatal—oxidative and/or alkylation injury. This injury can be prevented (in acetaminophen overdose, for example) by rapid treatment with NAC, an efficient non-toxic source of cysteine, which is able to replenish hepatocellular GSH. Chronic or slowly arising GSH deficiency due to administration of GSH-depleting drugs, or to diseases and conditions that deplete GSH, can be similarly debilitating[1].

The following discussion provides evidence for the development of cysteine/GSH deficiency in a variety of disease settings and considers the biochemical mechanisms through which this deficiency, and its correction, can impact disease processes.

Search and Inclusion Criteria

The following studies were selected by searching the PubMed database using the Endnote interface. Publications included here describe randomized placebo-controlled NAC trials testing N-acetylcysteine (NAC) for efficacy in a variety of disease settings, were located by a search strategy using the following keywords: placebo AND N-acetylcysteine AND NOT Animal. Abstracts were excluded, as were publications that were not in written in the English language. In addition, studies with fewer than 10 patients and/or unclear endpoints were excluded. All other publications that met the search criteria are included. The included studies are discussed below and summarized in table 1, in which we classify the findings according to whether they reports that NAC treatment results in 1) significant clinical benefit, 2) significant findings whose clinical relevance is unclear, or 3) no significant difference between the control and treatment groups.

Publications describing NAC trials that were not placebo-controlled, or describing observational NAC studies conducted with NAC, were located using the keyword N-acetylcysteine in combination with keywords identifying individual diseases. These trials and studies are not exhaustively reviewed here. Many, but not all, are listed by citation in table 2; some are discussed in later sections. We also discuss, but do not exhaustively review, findings from animal studies and from biochemical and other studies that provide information relevant to disease and GSH depletion mechanisms.

GSH Deficiency and Disease

A role for GSH deficiency in the clinical manifestations of a broad spectrum of diseases and conditions is suggested either by the direct documentation of low GSH levels in these conditions or by the demonstration of significant improvement in patient condition following NAC administration. Over fifty randomized placebo-controlled trials demonstrate beneficial effects of NAC treatment (table 1)[2-62] in diseases and conditions that include systemic inflammatory response syndrome (SIRS), Acute Respiratory Distress Syndrome (ARDS), chronic lung disease (CLD), chronic obstructive pulmonary disease (COPD), neurodegenerative disease, cardiovascular disease, alcoholism, infectious disease (e.g., HIV infection, chronic HCV hepatitis), hepatic and renal failure, diabetes, malnutrition and certain autoimmune diseases.

The mechanisms that underlie the development of GSH deficiency in disease are reasonably well understood, at least in some instances. A wide variety of inflammatory and metabolic stimuli common during active disease increase the production of intracellular oxidants. In addition, neutrophils and other cells present at sites of inflammation release oxidants (reactive oxygen and nitrogen intermediates) that enter other cells and add to the internal oxidant burden. GSH provides the main defense against toxic oxidative intermediates by reducing and thereby inactivating them. However, in so doing, GSH is oxidized to GSH disulfide (GSSG). GSSG is then either rapidly reduced to GSH by GSSG-reductase and NADPH or is excreted from the cell and only in part recovered from the circulation.

Factors that may contribute to GSH deficiency include GSH losses that occur when GSH is enzymatically conjugated to exogenous chemicals (drugs, dietary components and toxins) and excreted from the cell as GSH or acetylcysteine mercapturates (conjugates). In addition, disease processes may decrease the cellular uptake or synthesis of cysteine or cystine, increase GSH efflux[63], or increase the loss of cysteine/GSH sulfur due to accelerated oxidation to the final oxidized forms (sulfate and taurine)[64, 65]. Because a balance between cysteine supply and GSH utilization must be maintained, if oxidant production or levels of substrate for GSH conjugation are high and cysteine supplies for GSH replenishment become limiting, severe GSH deficiency may occur.

Importantly, there are significant potential iatrogenic contributions to GSH depletion. Inadvertent treatment with higher doses of acetaminophen than patients can tolerate is perhaps the most common. This can be particularly dangerous for patients with conditions in which GSH depletion tends to occur as a consequence of the disease process or following treatment with drugs that are detoxified by GSH. In addition, long-term maintenance on parenteral nutrition may result in GSH depletion since parenteral nutrition formulations are not necessarily designed to provide adequate cysteine equivalents to meet the metabolic needs of diseased patients. In the absence of adequate attention to maintenance of adequate cysteine supplies, physicians and other caregivers can inadvertently contribute to GSH deficiency.

Patient behavior may also result in the development of GSH deficiency. Chronic over-consumption of alcohol is well known to deplete GSH in certain tissues, particularly the liver, and thus to render patients susceptible to acetaminophen toxicity at doses well below those that cause toxicity in healthy individuals. Indeed, the FDA has issued a warning to this effect (http://www.fda.gov/ohrms/dockets/ac/O₂/briefing/3882b1.htm). However, chronic consumption of acetaminophen or other GSH-depleting drugs, even well below toxic dose levels, can gradually deplete GSH to the point where these drugs elicit toxicity. Such practices become more dangerous if patients are malnourished or are GSH deficient for other reasons.

In summary, GSH deficiency occurs more frequently than previously suspected. GSH is readily replenished by de novo synthesis as long as sufficient supplies of cysteine are available, either directly from dietary sources or indirectly by conversion of dietary methionine. However, failure to obtain sufficient dietary cysteine to replace that lost when GSH is oxidized or conjugated to drugs or exogenous chemicals results in cysteine, and hence GSH, deficiency (referred to here as cysteine/GSH deficiency) that may necessitate pharmacological intervention.

Dietary Sources of Cysteine

Cysteine utilized in the body is derived from dietary cysteine and methionine, sulfur-containing amino acids that are largely obtained from digested protein. Since mammals obtain cysteine both directly from the diet and by degradation of dietary methionine, the normal cysteine requirement can be satisfied from dietary sources. However, as indicated above, an additional source of cysteine may be required when cysteine loss (e.g., via GSH loss) outstrips the usual dietary supply.

Requirements for sulfur-containing amino acids in humans are based upon nitrogen and sulfur amino acid balance studies conducted with healthy individuals. The average American diet contains about 100 g of protein daily, greater than half of which is animal protein with a relatively high content of sulfur-containing amino acids. The recommended daily allowance (RDA) for sulfur amino acids (SAA) for an adult male is about 1 g (200 mg of methionine and an additional 810 mg methionine that can be replaced by an equivalent amount of cysteine[66]). A healthy, well-fed person will often consume greater than twice the sulfur amino acid RDA. However, poor appetite and/or a tendency to select fresh food with low sulfur amino acid content or bioavailability[67] or processed food depleted of sulfur amino acids[68-70] can result in cysteine deficiency even in otherwise healthy people. Furthermore, as evidence here indicates, the need for sulfur amino acids can be substantially increased in many disease states.

The limited ability of the body to store amino acids is an additional problem. The human liver does contain a reservoir of cysteine (about 1 g) that is largely present in GSH. Since this amount approximates the daily sulfur amino acid requirement, it provides only a short-term source to maintain a stable cysteine supply despite intermittent methionine and cysteine consumption. Under conditions of excessive cysteine requirements or deficient cysteine/methionine consumption, GSH is also released from skeletal muscle and other tissues to supply cysteine. This results in decreased antioxidant and detoxification functions throughout the body. Consequently, even short term inadequate intake of sulfur amino acids can pose a risk to individuals who may consume adequate amounts most of the time[71, 72].

Mechanisms that May Mediate the Clinical Effects of Cysteine/GSH Deficiency

GSH has multiple roles in cells, ranging from neutralization of (ROI) to acting as a co-enzyme in a variety of metabolic processes. The widespread participation of GSH in biochemical reactions of importance to cell growth, differentiation and function offers mechanistic insights into how interfering with GSH homeostasis could influence the course of varied disease processes. A full discussion of the preclinical data bearing on these issues is beyond the present scope. However, to provide a mechanistic context for the clinical findings we discuss, we have summarized some of the key processes regulated by GSH:

Oxidative reactions. In its most well-known role, GSH participates in enzyme mediated reactions to neutralize ROI and thus prevents the accumulation of ROI damage to DNA, proteins and lipids. Glutathione peroxidases play a key role in this process by catalyzing the reaction of GSH with peroxides, including hydrogen peroxide and lipid peroxides. Thus, decreasing GSH can sharply augment oxidative damage and result in cell death or loss of function.

DNA synthesis. Low GSH availability can impair DNA synthesis since GSH acts (via glutaredoxin) as a coenzyme for ribonucleotide reductase, an enzyme required for the synthesis of deoxyribonucleotides[73-75].

Gene expression and signal transduction. GSH has been shown to regulate or influence the expression of several genes, notably inflammatory genes under the control of transcription factors neucular factor kappa B (NF-□B) and activator protein 1 (AP-1), even in settings where there is no marked overproduction of ROI. In addition, GSH has been shown to regulate T cell signaling by controlling phosphorylation of phospholipase C (PLC)□ 1, which is required to stimulate the calcium flux that occurs early in the T cell receptor signaling cascade[76, 77]. Importantly, GSH has also been shown to regulate the expression of vascular cell adhesion molecule-1 (VCAM-1) on vascular endothelial cells, one of the early features in the pathogenesis of atherosclerosis and other inflammatory diseases[21, 78-81].

Enzymes and protein functions. GSH regulates the activity of enzymes and other intracellular molecules by post-translational modifications (glutathionylations) that control the oxidation state of protein-SH groups. When intracellular GSH is at its normal level for a particular cell type in a healthy individual, most of the free protein thiol groups are reduced, i.e., are present as protein-SH. In contrast, when GSH levels are low and/or GSSG levels are increased, GSH is reversibly coupled to many free thiols to create mixed disulfides (protein-S-S-G)[82]. These S-glutathionylated proteins, which may be functionally altered, then persist as such until GSH levels return to normal.

By controlling the activities of a series of enzymes and other intracellular proteins, glutathionylation can rapidly and reversibly alter the metabolic status of cells in response to changes in the redox environment. For example, glutathionylation has been shown to regulate actin polymerization[83], to inhibit the activity of several key enzymes (including glyceraldehyde-3-phosphate dehydrogenase, carbonic anhydrase and protein tyrosine phosphatase) and to activates or stabilize other enzymes (including HIV-1 protease and the NF-□B transcription factor[84]). Nitrosylation of protein thiols has similarly been shown to increase under oxidative conditions[85-88] and to alter functions of key enzymes[89] and other molecules[90-92]. Thus, both glutathionylation and nitrosylation are of central importance to mechanisms through which cysteine/GSH deficiency may impact cell, and hence organ, function.

As indicated above, these types of post-translational modifications are highly sensitive to shifts in the intracellular redox balance. They are rapidly initiated when GSH is depleted and rapidly reversed when GSH is replenished. As such, they provide the kind of flexible response to oxidative stress necessary for organisms living in an oxidative environment. However, at the extreme, they may underlie some of the pathologic changes that occur when chronic cysteine/GSH deficiency occurs in disease.

Glutaredoxin and thioredoxin. Glutaredoxin (Grx) and thioredoxin (Trx) belong to the two major disulfide reductase enzyme families, which take electrons from GSH and NADPH via Grx reductase and Trx reductase, respectively[73-75, 93-95].

Trx and Grx interact with proteins to regulate functional activity, both directly and via glutathionylation. Intracellular GSH and GSSG levels play a major role in this regulation. The activity of Grx is directly regulated by the amount of intracellular GSH and GSSG, which controls the status of the Grx active site. The active sites in Trx (Cys-Gly-Pro-Cys) and Grx (Cys-Pro-Tyr-Cys) contain a dithiol that can be oxidized when GSH levels are low (or GSSG levels increase) to form an internal disulfide between the two cysteine residues or a mixed disulfide in which GSH is bound to one or both cysteine residues in the active site. Formation of the Grx mixed disulfide[82, 93, 96] represents a special case of protein glutathionylation since it arms the Grx for glutathionylation of other proteins. Although Trx can also be glutathionylated[96], current data indicate that glutathionylation is mainly mediated by the Grx mixed disulfide[97-99].

Oxidation of Grx and Trx active sites can also regulate Trx or Grx functions mediated by direct binding to key intracellular proteins. For example, under reducing conditions, Trx and Grx protect cells from apoptosis by binding to and inactivating the apoptosis-signaling kinase I[99, 100] whereas this binding is blocked and apoptosis induction proceeds at low GSH levels (and/or high GSSG levels)[74, 75].

Selenoenzymes. Decreasing GSH increases the intracellular redox potential of the GSH/GSSG couple and puts an additional burden on the thioredoxin-thioredoxin reductase system. This may be quite important in patients who have low selenium levels, since human thioredoxin reductases are selenoenzymes with an essential selenocysteine residue in the active site[75, 101-105]. Cysteine/GSH deficiency in these patients, in whom thioredoxin reductase activity is compromised, may make them particularly susceptible to cell damage under oxidative stress.

Thus, cysteine/GSH deficiency can impact cell and organ function through multiple pathways operating at the same or different sites, depending on the underlying mechanisms responsible for depleting GSH. This potential for affecting different pathways in different diseases perhaps explains why the effects of cysteine/GSH deficiency have not been readily recognizable as a single clinical entity.

N-Acetylcysteine (NAC) Treatment to Relieve Cysteine/GSH Deficiency

Clinical experience in the treatment of acetaminophen toxicity has established that rapid administration of NAC, an essentially non-toxic cysteine source, restores normal GSH levels in solid tissues and the systemic circulation and thus prevents the potentially lethal consequences of severe cysteine/GSH deficiency induced by acetaminophen overdose. In addition to this well-known role for NAC, however, NAC treatment has been shown to be clinically beneficial in a wide variety of diseases and conditions. In fact, over fifty randomized placebo-controlled trials (table 1) have reported beneficial effects of NAC treatment. Collectively, these studies demonstrate that cysteine/GSH deficiency is an important emerging clinical entity and that NAC administration offers an effective method for treating this deficiency.

Although various forms of cysteine and its precursors have been used as nutritional and therapeutic sources of cysteine, NAC is the most widely used and extensively studied*. NAC is about 10 times more stable than cysteine and much more soluble than the stable cysteine disulfide, cystine. L-2-oxothiazolidine-4-carboxylate (procysteine/OTC) has also been used effectively in some studies[106] as have glutathione and glutathione monoethyl ester[107]. In addition, dietary methionine is an effective source of cysteine, as is S-adenosylmethionine (referred to either as SAM or SAM-e)[108]. We focus on NAC in this review because NAC is the cysteine source for used correcting cysteine/GSH deficiency in most studies and because NAC is already approved for therapeutic use for treatment of acetaminophen overdose and as a mucolytic agent in cystic fibrosis.

Surprisingly, given the diverse roles that GSH plays in cellular physiology and regulation of enzyme activity and protein function (see above), GSH deficiency has mainly been discussed from a clinical perspective in terms of the loss of intracellular protection against oxidative stress. Similarly, NAC is principally considered to be an antioxidant rather than a source of cysteine for GSH replenishment. However, while antioxidants such as vitamin E and vitamin C can spare GSH under conditions of oxidative stress, GSH loss due to oxidative or detoxifying reactions can only be offset by GSH resynthesis, which requires a cysteine source.

Cysteine/GSH Deficiency and NAC Therapy in Disease Settings

In the sections that follow, we discuss reported outcomes of NAC therapy in various clinical settings. We largely focus on findings from the randomized placebo-controlled studies (table 1), but also discuss selected findings from observational studies that further illuminate clinical aspects of cysteine/GSH deficiency. Citations for these and other observational NAC treatment studies are presented in table 2, which provides a partial survey of the literature in this area.

Acetaminophen Toxicity.

The toxicity of acetaminophen is due to depletion of GSH in hepatocytes“ ” [109-114]. Acute dose levels of acetaminophen likely to cause severe liver toxicity are well established for healthy individuals[110]. However, under conditions in which GSH levels are compromised, doses of acetaminophen that are within the usual prescribed range can also cause hepatic injury and failure[71, 110]. Thus, acetaminophen usage, and the usage of other GSH-depleting drugs, may be quite important to overall pathology in diseases and conditions such as those discussed in the section that follow, where GSH deficiency is known to occur.

Hepatic and Gastrointestinal Disease.

Hepatic failure (Table 1a). Acetaminophen overdose is a well-known cause of fulminant hepatic failure. In fact, a recent study indicates that acetaminophen overdose and idiosyncratic drug reactions have now replaced viral hepatitis as the most frequent causes of acute liver failure in the United States[115]. N-acetylcysteine (NAC), which provides the cysteine necessary to replenish GSH depleted by toxic agents, is extremely effective in preventing liver damage due to acetaminophen toxicity. Although placebo-controlled studies demonstrating this point are scarse (one report), NAC administered promptly and at a sufficient dose^{e.g.,}[2, 71, 109, 110, 113, 116-159] has been the standard of care for treatment of acetaminophen poisoning for many years[110]. In addition, NAC has been suggested as treatment for Amanita phalloides poisoning[160] and for other exposures to toxic agents^{e.g.,}[161-163].

The importance of administering NAC within the first twenty-four hours of acetaminophen overdose is clear. However, later administration of NAC has also been shown to be effective. In a placebo-controlled study of patients with acetaminophen-induced fulminant hepatic failure who had not previously received NAC, both trial arms showed similar rates of deterioration and recovery of liver function (table 1a). However, subjects in the NAC arm showed a significant increase in survival and a corresponding decrease in the incidence of cerebral edema and hypotension requiring inotropic support [2].

Consistant with these findings, an observational study with one hundred patients who have already developed fulminant hepatic failure due to untreated acetaminophen overdose showed significantly increased survival in the NAC-treated group[138]. A subsequent study, also observational, showed that NAC treatment significantly improves cardiac index, mean oxygen delivery, mean arterial pressure, oxygen consumption and oxygen extraction ratio in patients with fulminant hepatic failure due either to untreated acetaminophen overdose or to other causes[164].

The chronic consumption of alcohol poses a special risk with respect to acetaminophen overdose[139, 147] because alcoholic patients often have lower GSH levels. In such patients, doses of acetaminophen below those usually considered toxic can be expected to deplete GSH below the critical threshold for hepatocellular necrosis[165]. Thus, a recent study suggests that due to an increased risk of developing hepatotoxicity, patients with chronic alcoholism and suspected acetaminophen poisoning should be treated with NAC regardless of risk estimation[166-168].

The US Food and Drug Administration (FDA) recognized the importance of alcohol consumption in predisposing to acetaminophen toxicity by ruling (in 1998) that acetaminophen package labels must include the following warning: “If you consume three or more alcoholic drinks every day, ask your doctor whether you should take acetaminophen or other pain relievers/reducers. Acetaminophen may cause liver damage.” The reasons underlying this ruling are reviewed in a report accessible at <http://www.fda.gov/ohrms/dockets/ac/02/briefing/3882b1 01 E-Final%20Rule.pdf>.

Gastrointestinal disease. Two randomized, placebo-controlled studies showed beneficial effects of NAC treatment on GI-related pathophysiology (see table 1a). In one study conducted as part of an effort to develop chemoprevention for carcinogenesis of the large bowel, NAC treatment was found to lower the proliferative index in the colonic crypt epithelium of subjects who previously had adenomatous polyps [55]. In the second study, NAC treatment in the presence of hyperoxic ventilation better preserved end-organ oxygen utilization and was associated with improved gastric intra-mucosal pH.

A published abstract of a placebo-controlled trial indicated that NAC treatment decreased the sensitivity and specificity of the Helicobacter pylori stool antigen test. However, we were unable to obtain the full text article for evaluation of this conclusion.

Protein-Energy Malnutrition (PEM). Several studies have demonstrated GSH depletion in children with the edematous syndromes of PEM, kwashiorkor and marasmic-kwashiorkor[54, 169-171]. Children with edematous PEM also have elevated levels of biomarkers of cellular oxidant damage[172, 173], indicating that a greater sensitivity to the effects of free radicals on cellular components. The observation that levels of biomarkers of oxidant damage normalize as soon as clinical signs and symptoms resolve[172] suggests that oxidant damage plays an important role in the pathogenesis of the disease.

In a study of children with edematous PEM, Jahoor and colleagues showed that erythrocyte GSH depletion is due to a slower rate of synthesis secondary to inadequate cysteine availability[171]. In another more recent study of a similar group of children with edematous PEM, Jahoor and colleagues demonstrated that GSH synthesis rate and concentration can be restored during the early phase of nutritional rehabilitation if diets are supplemented with NAC[54]. The observation that edema is lost at a faster rate by the group whose GSH pools were restored early with NAC suggests that early restoration of GSH homeostasis in children with edematous PEM accelerates recovery. This possibility is supported by another study showing that increases in GSH levels in children with kwashiorkor are associated with recovery[173].

These findings also raise the question of whether the modest malnutrition common in elderly people, who also frequently have low GSH levels[174], puts the elderly at risk for developing clinically significant cysteine/GSH deficiency and hence at increased risk of hepatic and other tissue injury associated with consumption of GSH-depleting pharmaceuticals such as acetaminophen.

Cardiovascular Disease

Nine placebo-controlled studies showed beneficial effects of NAC treatment in cardiovascular disease (see table 1b). NAC treatment enhanced the coronary vasodilator effects of nitroglycerin in patients with unstable angina, yeilding significant clinical benefit[13, 14, 159, 175-178]. In addition, several placebo-controlled studies demonstrated that treatment with NAC decreased development of nitrate tolerance in patients with stable angina[13, 14, 159, 177]. Nevertheless, the use of NAC in combination with nitroglycerin is limited because it is frequently associated with severe headache[177], most likely due to enhancement of the vasodilator effect.

NAC treatment was also shown to be effective as preventative cardiac measure. A placebo-controlled study evaluating NAC pretreatment in cardiac risk patients examined during periods of hyperoxia showed that the pretreatment attenuated tissue oxygenation impairment and preserved myocardial performance better pretreatment with placebo [11]. NAC treatment in a randomized, placebo-controlled study of 134 chronic hemodialysis patients who are at high risk for cardiovascular events also shows that NAC treatment is associated with a significant decrease (RR 0.60, p=0.03) in composite cardiovascular outcome, including occurrence of myocardial infarction, need for coronary angioplasty or coronary artery bypass surgery, ischemic stroke, or peripheral vascular disease[17]. The NAC-treated group in this study also had much lower serum oxidized LDL values at the end of the study.

In an observational trial with patients undergoing thrombolytic therapy for myocardial ischemia, 24-hour intravenous infusion of GSH significantly decreased the oxidative stress of reperfusion injury as demonstrated by decreased plasma malondialdehyde (MDA) [179]. The GSH-treated patients also had fewer episodes of arrhythmia.

Renal Disease.

Acute renal failure. Nephrotoxicity from acute acetaminophen overdose in the absence of hepatic toxicity has only rarely been described[148, 180]. However, analgesic nephropathy associated with chronic use of compounds containing phenacetin (which is metabolized to acetaminophen) and other non-narcotic analgesic products has been recognized as a cause of renal failure for years[181, 182]. The percentage of incident cases of end-stage kidney failure due to analgesic nephropathy as the principal cause varies from 1% to over 10%, depending on the country considered. In addition, chronic use of these analgesics may contribute to the risk of kidney failure in individuals with chronic kidney injury from other causes. Several large studies have estimated the relative risk of development of kidney failure as a function of analgesic consumption[183, 184]. Most recently, Føred and colleagues reported a dose-dependent increase in the risk of chronic renal failure associated with chronic exposure to acetaminophen in the absence of aspirin use. These investigators showed that the relative risk of chronic renal failure was 5.3 for regular users of acetaminophen consuming ≧1.4 g/day on average[1 84].

Contrast-induced nephropathy (CIN). Six of twelve placebo-controlled studies showed beneficial effects of NAC treatment in CIN (see table 1c). Tepel and colleagues have reported evidence from a placebo-controlled study implicating GSH deficiency in the pathogenesis of contrast nephropathy, a form of acute renal failure[3-5]. These investigators compared NAC to placebo in a study of 83 patients with baseline renal impairment undergoing computed tomography with a nonionic low-osmolality contrast agent. They demonstrated a 90% reduction in the incidence of acute renal failure in subjects treated with NAC, suggesting that GSH replenishment is protective in this clinical setting.

A total of twelve placebo-controlled studies with similar design have been reported[3, 6-10, 185-189]. Five of these[6-10] found about the same level of protection (RR* 0.13 to 0.32) reported by Tepel[3]. Another study, with 183 patients, demonstrated a significant reduction in the incidence of contrast nephropathy (from 8.5% to 0%, p=0.02) only in the subset of patients receiving lower volumes (<140 mL) of contrast agent[190], and yet another[189] showed borderline statistically significant protection for the subgroup of patients with the lowest pre-contrast renal function (creatinine clearances: 30-59 mL/min/1.73 m2). One study [9] addressed the mechanisms by which NAC treatment may protect kidney function and reported increased renal nitric oxide (NO) production in the NAC treated subjects. However, four studies failed to show any protective effect of NAC in preventing contrast nephropathy[185-188]. An additional study using an abbreviated intravenous NAC protocol showed a beneficial effect[191].
*Relative Risk

The striking differences in outcomes (considerable decreases in relative risk vs absence of any protection) in these large, well-designed studies is puzzling, inasmuch as most of the study designs were similar with respect to the degree of renal impairment, percentage of patients with diabetes mellitus, use of saline loading and of nonionic, low-osmolality contrast agents, and the doses of NAC used. The incidence of contrast-induced nephropathy in the placebo-treated groups of the studies that showed protection tended to be higher than in those that did not (23±14% vs 12±6%, P=0.11) although the difference did not achieve statistical significance, suggesting that the difference in outcomes is probably not due predominantly to differences in host factors.

In some negative studies, the follow-up time may have been insufficient to demonstrate the full effects of NAC treatment [189]. In addition, since the source of the NAC used was not stated in most of the studies, it is difficult to exclude differences in NAC potency (related to different sources, vide infra) or the presence of contaminants as a possible factor in the outcomes. Thus, there is currently no obvious explanation for the different findings in these studies. Even so, the majority of the published studies support a strong protective effect of NAC in preventing contrast-induced nephropathy.

Three metaanalyses have recently been published in this highly active area. All found significant heterogeneity among the studies reviewed. Two interpreted the studies to show evidence for reduction in the risk of contrast-induced nephropathy with use of NAC[192, 193]. One concluded that the heterogeneity in the studies precluded aggregation of the data as needed to complete the metaanalysis[194]. Two of the studies recommended initiation of a large randomized placebo-controlled trial to address the efficacy of NAC in this setting[193, 194].

Kidney transplantation Delayed graft function (DGF) after kidney transplantation is probably in large part caused by production of ROI and other reactive species following reperfusion of the transplant organ after a period of warm and cold ischemia. In general, these reactive molecules are detoxified by GSH-dependent mechanisms, including conjugation to GSH by a family of GSH-S-transferase (GST) enzymes, some of which are expressed in large quantity in the proximal tubule of the kidney[195]. In an observational study of 229 kidney transplant recipients, donor (but not recipient) GST MIB polymorphism was associated with significantly lower rates (RR 0.33) of DGF after transplantation[196].

Lack of association with other GST alleles in this study complicates the interpretation of the specific role(s) GST may play in reducing DGF risk. However, since the association was only observed when the protective allele was carried by the transplanted organ, there is reason to suspect that a particular GST (or a closely linked protein) may be protective in this setting.

Endocrine Disease (Insulin Sensitivity)

Three randomized placebo-controlled trials demonstrate beneficial effects of NAC treatment in insulin-related disease (see table 1d). One study demonstrates that oral administration of NAC to patients with non-insulin dependent diabetes mellitus (NIDDM) reverses the elevation of soluble vascular cell adhesion molecule-1 (VCAM-1) [21], a substance which promotes accumulation of macrophages and T-lymphocytes at sites of inflammation and increases progression of vascular damage[79, 80]. A second placebo-controlled study by the same group shows that intravenous GSH infusion significantly increases both intraerythrocytic GSH/GSSG ratio and total glucose uptake in NIDDM patients and suggests that abnormal intracellular GSH redox status in these patients plays an important role in reducing insulin sensitivity[22]. Consistent with these findings, in an ongoing study in type 2 diabetics, Jahoor and colleagues have demonstrated that two weeks of dietary supplementation with NAC elicited significant increases in both erythrocyte GSH concentration and the rate of its synthesis, suggesting that positive clinical effects of NAC are mediated through improved GSH availability[197].

Finally, a study of patients with hyperinsulism secondary to polycystic ovary disease demonstrates that NAC treatment significantly decreases serum insulin levels and insulin sensitivity without altering fasting glucose levels [20]. The authors conclude that NAC may be a new treatment for the improvement of circulating insulin levels and insulin sensitivity in these patients.

Metabolic and Genetic Disease.

Genetic defects that impair GSH synthesis or homeostasis are well known (reviewed by Ristoff[198]). The most common defect affects GSH synthetase (GS) and has a wide range of disease manifestations, including hemolytic anemia, progressive neurological symptoms, metabolic acidosis and, in the most severe form, death during the neonatal period. Data from a small observational study suggests that early supplementation with Vitamins C and E may improve long-term outcome in these patients[199].

Cystic fibrosis. Two placebo-controlled studies report beneficial effects of NAC treatment on lung function in cystic fibrosis (table 1e). A third study reports improvement in measures of lung function, but saw no significant clinical differences between NAC and placebo arm subjects. A very short fourth study (2 weeks) failed to find any significant difference between the trial arms.

Sickle cell disease. In a placebo-controlled trial examining NAC treatment in sickle cell disease, NAC was found to inhibit dense cell formation and decrease the number of episodes of vaso-occlusive disease. These improvements in patient condition were associated with increase in glutathione levels.

Homocysteine metabolism. Two of three NAC randomized, placebo-controlled trials showed beneficial effects of NAC treatment in patients with elevated homocysteine (see table 1e). NAC significantly increased homocysteine removal by hemodialysis. This reduction in plasma homocysteine was significantly correlated with reduction in pulse pressure and improved endothelial function. In a second trial, NAC decreased plasma homocysteine levels in patients with elevated plasma lipoprotein A.

Pulmonary Disease

Chronic lung disease/Chronic Obstructive Pulmonary Disease (CLD/COPD) (see table 1f). In twelve of eighteen randomized, placebo-controlled trials, NAC treated showed beneficial effects for patients with forms of chronic lung disease. Patients to whom NAC was administered orally demonstrated decreased days of illness, improved response to steroids, decreased disease exacerbation rates and a general increase in well-being[23-34]. One study showed a benefit whose clinical relevance is unclear and seven failed to find significant benefit[200-204]. Metaanalyses of the bronchitis studies showed that NAC treatment significantly decreases the costs of hospitalizations, emergency room visits, medications, and work time lost [205-210].

Beneficial effects of NAC treatment have been demonstrated in COPD patients, who showed significant improvements in Forced Expiratory Volume during the first second (FEVI) and Maximum Expiratory Flow (MEF50)[210]. Mucociliary clearance in COPD subjects and in otherwise healthy smokers also showed improvement[36] as did modulation of cancer-associated biomarkers in specific organs in smokers[35];

Systemic Inflammatory Response Syndrome (SIRS) and Acute Lung Injury (ALI); multiple organ system failure (MOSF)(see tables 1f-h). Five of seven randomized placebo-controlled studies showed beneficial effects of NAC as adjunct therapy acute lung injury and end-organ failure. RFesults of these studies indicate that oxidative stress and cysteine/GSH depletion play a major role in inflammation leading to capillary leak syndromes and end-organ failure[44, 46, 211]. These conclusion is supported and partially explained by evidence showing that 1) NAC decreases the cytotoxic effects of TNF-□ and other inflammatory cytokines[212]; 2) NAC decreases neutrophil elastase production in acute lung injury[213-217] and, 3) NAC increases neutrophil protection and decreases mortality in cecal ligation and puncture septic shock[218].

In ALI, results from randomized, placebo controlled trials demonstrate significant antioxidant effects and improved outcome[11, 42, 44, 211, 215, 219-223]. Data from these trials show that NAC treatment decreases the level of respiratory distress, the work of breathing and number of days of mechanical ventilation. In addition, data from the trials show that NAC treatment improves static lung compliance, oxygenation measured either as oxygen index or partial pressure of arterial oxygen (PaO₂), and sustained diaphragmatic muscle activity[211, 219, 222, 223].

Three studies failed to demonstrate significant benefit of NAC treatment in ALI[45, 219, 224], most likely because of the timing of NAC administration. In the studies in which NAC was found to be beneficial [11, 42, 44, 211, 215, 219-223], treatment was initiated 24-48 hours and maintained for 3-10 days after the inciting event. In contrast, in the studies in which no benefit was associated with NAC treatment[45, 219, 224], the treatment was either terminated before day 3 of illness or begun after chronic lung disease with ventilator dependence was well established.

Septic Shock and Infectious Disease.

Septic shock. Three of four randomized placebo-controlled trials demonstrate beneficial effects of NAC treatment in septic shock, as demonstrated by improved tissue oxygenation, decreased veno-arterial PCO₂, decreased IL-8 production, increased cardiac index and overall increased survival. One study, however, demonstrated progressive decrease in mean arterial pressure, cardiac index and stroke work (see table 1g). In this latter study, which is listed as adverse in table 1g, NAC was administered earlier than the preceding studies (two hours after hemodynamic stabilization), which may have confounded the endpoint. In any event, findings from this study suggest that the timing of NAC administration requires further investigation.

HIV disease. A broad series of studies clearly demonstrates GSH levels in erythrocytes, lymphocytes and other peripheral blood mononuclear cells progressively decrease as HIV disease advances[47-49, 225-228]. In addition, careful pharmacokinetic studies demonstrate that the low GSH in HIV-infected individuals is due to limited availability of sufficient cysteine to maintain cellular GSH homeostasis[229, 230]. In fact, a massive peripheral tissue catabolism of sulfur-containing peptides and amino acids has been observed in HIV patients[64, 65].

Five of six randomized placebo-controlled studies show beneficial effects of NAC treatment in HIV infection. Several trials collectively demonstrated that NAC administration to HIV-infected subjects with low GSH levels replenishes lymphocyte and erythrocyte GSH (see table 1 g) [48, 50]. Importantly, one of these studies demonstrates that NAC treatment significantly improves T cell function[50]. This finding supports the idea that cysteine/GSH deficiency contributes to the immunodeficiency in HIV-infected individuals and plays an important and reversible role in the functional impairment of those T cells that are still present at later stages of HIV disease.

Cysteine/GSH deficiency may also contribute to the failure of the innate immune system and the development of opportunistic infections in the final stages of HIV disease. Observational studies have shown that HIV-infected individuals with low CD4 T cell counts and low cellular and systemic GSH levels frequently have elevated blood levels of thioredoxin (Trx), which is an effective chemokine[231]. In mice, circulating Trx (like other chemokines) blocks neutrophil migration to infection sites and hence interferes with innate defense against invading pathogens[218]. Similar interference may occur in HIV infection, since the survival of infected individuals with Trx levels above the normal range is significantly decreased compared to survival of subjects with Trx levels in the normal range[82]. Since NAC treatment lowers Trx levels[94, 232] this may contribute to the observed association between NAC ingestion and prolonged survival in HIV disease[47, 48, 227, 233].

The improvement in T cell function observed in HIV-infected subjects treated with NAC[50] suggests that NAC treatment may be a useful adjunct in HIV vaccination. In addition, this improvement provides a rationale for the strong associations observed between low GSH levels and decreased survival in HIV infection[49] and between NAC administration and improved survival in an open-label NAC study[234].

Influenza. One randomized placebo-controlled trial demonstrated that long-term therapy with oral NAC during “cold” season significantly attenuated the frequency and severity of influenza episodes in elderly subjects and in patients suffering from chronic non-respiratory diseases.

Malaria. NAC (300 mg/kg given intravenously over 20 hours) was tested as adjunctive therapy for severe malaria in two double-blind, placebo-controlled studies[51],[52]. Results from one study (30 subjects[51]) showed that elevated serum lactate levels, an indication of disease severity in potentially life-threatening malaria[235-237], returned to normal twice as quickly in the patients who received NAC than in those who received placebo. Summary of results from a second study (108 subjects [52]) indicate strong positive findings, leading the authors to a call for a large double blind trial of NAC as an adjunctive therapy for severe malaria. (Unfortunately, we were unable to obtain the full text article describing this latter study.)

The mechanism(s) through which NAC produced beneficial effects in these studies were not defined. However, several key processes in falciparum malaria are potential NAC targets. For example, NAC is known to decrease the activity of TNF-□, which has been shown to mediate cerebral dysfunction in murine cerebral malaria[238] and has been implicated in the pathogenesis of severe human malaria[239]. In addition, NAC may act by preventing adherence of Plasmodium falciparum-infected red blood cells to CD36 on postcapillary venular endothelium, an important step in the pathogenesis of severe malaria[240]. Finally, NAC may play a direct role in decreasing serum lactate production by replenishing GSH and thereby reversing the loss of glyceraldehyde phosphate dehydrogenase activity and the shift to glycolytic metabolism likely to occur when GSH is depleted[82].

Multiorgan Failure (table 1h).

NAC treatment for SIRS and MOSF has not been very well studied. One randomized, placebo-controlled study demonstrated that NAC improves ex vivo phagocytosis activity. [220] Three additional studies show a NAC-associated increase in cardiac index and decrease in systemic vascular resistance but have conflicting results with respect to oxygen delivery, oxygen extraction and serum lactate levels [44, 45, 222]. None of these studies report a significant difference in mortality between the NAC and control groups. However, all of the studies were relatively small and had heterogenous SIRS etiologies, suggesting that larger, better-controlled studies are required before conclusions can be drawn with respect to the clinical benefits of NAC treatment in SIRS and MOSF.

The only side effect reported for NAC treatment for inflammatory conditions is mild to moderate anticoagulation[106]. This may be attributable to antioxidant-mediated release of nitric oxide (NO) from sequestered peroxynitrate. It was not found to be clinically significant.

Neurologic and Musculoskeletal Disease (table 1i)

Evidence implicating GSH deficiency in a series of neurogenerative diseases is summarized in a recent review[241] by Schulz and colleagues, who also discuss the use of NAC and other drugs as potential therapeutic approaches. Few randomized placebo-controlled trials have been conducted in this area (table 1i). One, testing NAC treatment in Alzheimer's disease, reported significant benefits for some outcome measures and favorable trends for others[60]. Another, testing NAC treatment in amyotrophic lateral sclerosis, found no significant benefit[242].

NAC treatment was also shown to be beneficial for frail geriatric patients responding to exercise in that it significantly enhanced knee extensor strength and increased the sum of all strength parameters.

Otolaryngolic and Opthamalogic Disease.

Otic disease. Data from a randomized placebo-controlled trial demonstrate that NAC improves response to treatment in otitis media with effusion (OME), a sustained non-specific inflammation of the middle ear mucosa accompanied by secretory transformation of the epithelium and accumulation of fluid in the middle ear space[53] (see table 1j). Instillation of NAC in the middle ear in children who were undergoing their first bilateral insertion of ventilation tubes (VT) due to OME, followed by NAC instillation at day 3 and 7 after VT insertion, significantly decreased OME recurrence, increased the time until VT extrusion (p<0.0167) and decreased VT re-insertions (p<0.025). In addition, the number of episodes of ear problems and visits at the ENT clinic were significantly decreased in the NAC-treated children (p<0.0383).

Pre-clinical studies also point to the importance of oxidative stress and GSH depletion in the genesis of noise and toxin-induced hearing loss[243, 244]. Medications with inner ear toxicity such as aminoglycoside antibiotics and the chemotherapy agent cisplatin have been shown to damage the cochlea through the generation of oxygen free radicals. Hearing loss and cochlear damage associated with administration of these compounds has been shown, in animal models, to be greatly reduced by administration of both NAC and methionine[245-247]. Similarly, studies with animal models show that permanent cochlear damage due to acute acoustic overexposure, which induces ischemia reperfusion, glutamate excitotoxicity, free radical generation and GSH depletion[243, 244, 246, 248], can be almost completely prevented by systemic administration of NAC or methionine[246, 248]. Thus, the stage is now set for clinical trials using NAC or similar agents to prevent or reverse the acute cochlear injury associated with noise or a variety of ototoxins.

Ocular and oral disease. Beneficial effects have been demonstrated following NAC treatment in placebo-controlled studies in chronic blepharitis and in Sjogren's syndrome, where improvements in ocular soreness, ocular irritability, halitosis and daytime thirst were noted. In addition, NAC treatment has been shown to significantly decreas dental plaque formation (table 1j).

Dermatologic Disease (table 1k).

A placebo-controlled trial examining NAC treatment in progressive systemic sclerosis failed to find significant effects of NAC treatment.

Therapeutic Dosing with NAC

NAC is the clinically accepted cysteine source used to treat GSH deficiency due to acetaminophen overdose. It can be administered by intravenous, enteral, and rectal routes. Oral NAC dosages for acetaminophen overdose start with a loading dose of 140 mg/kg body weight followed by doses of 70 mg/kg body weight administered every four hours over a period of three days[129]. Smaller dosages (600 mg-8 g daily) have been administered for substantially longer periods in clinical trials for other conditions (table 1).

Although NAC has been administered orally at quite high dosages, little if any toxicity has been associated with NAC ingestion. The highest reported long-term NAC dosage (an average of 6.9 g/day administered in 3-4 divided doses) was administered to 60 HIV-infected subjects for eight weeks during a placebo-controlled trial and to over 50 subjects for up to six months during the open-label continuation trial[49]. No adverse events requiring physician intervention were observed during either trial segment. During the placebo-controlled segment of the trial, 14/60 subjects reported gastric distress similar to that reported elsewhere[48, 49]. However, half of these subjects (7/14) were in the placebo arm, suggesting that the distress was related to ingestion of the excipient which may have contained significant amounts of lactose.

There are several reports of anaphylactoid and allergic responses in response to intravenous NAC administration[163, 249-256]. These adverse events may in part be explained by findings from preclinical studies demonstrating inflammatory-type responses in animals treated with the oxidized (dimeric) form of NAC[257] which can contaminate NAC preparations that have not been protected against oxidation (see below). In any event, the anaphylactoid reactions to intravenous NAC are easily treated and do not interfere with completion of NAC administration[254].

Finally, caution may be indicated concerning the routine consumption of NAC and other sulfur amino acid precursors in the absence of diseases or conditions leading to cysteine/GSH deficiency, particularly in American and other populations in which the intake animal protein tends to be high and individuals may be ingesting 2-3 times the RDA for sulfur amino acids on a daily basis. We have shown that long-term administration of roughly 6 g/day of NAC to HIV-infected individuals is safe[48, 49]. However, recent observational studies showing that colonic hydrogen sulfide production increases in proportion to consumption of animal protein raises questions[258] as to whether increasing sulfur amino acid intake might predispose toward ulcerative colitis. While the role of hydrogen sulfide in ulcerative colitis is still controversial[259-261], it is important to bear in mind that the long-term risks of routinely increasing sulfur amino acid intake in healthy individuals have not yet been evaluated. Current evidence suggests that 600-900 mg/day, the common daily dosage in Europe for cough and cold relief, may be a reasonable maximum dose for healthy individuals who wish to routinely take NAC.

NAC Formulations

The best known NAC formulation in the US, Mucomyst™ (or the generic version thereof), is available as a 10% or 20% solution of NAC sodium salt that is typically administered orally for treatment of acetaminophen overdose. Since Mucomyst has a strong, disagreeable flavor, it is usually mixed with fruit juice or a soft drink before consumption. Still, as many physicians can attest, patients commonly find it very difficult to tolerate orally, thereby requiring administration via nasogastric tube. Mucomyst is also administered intravenously in some settings, particularly when patients are unconscious or unable to retain the orally administered drug.

To overcome problems with oral administration, European manufacturers produce NAC in pill and capsule formulations. It is also produced and packaged in a variety of effervescent formulations (“fizzy tabs”) that can be dissolved in water, juice or carbonated drinks to create a pleasant tasting, readily tolerated beverage. These formulations are produced under European Good Manufacturing Practice (GMP) standards designed to minimize NAC oxidation to its dimeric form (“di-NAC”), which is pharmacologically active at very low concentrations with immunologic effects opposite to those of NAC[257]. In general, di-NAC constitutes less than 0.1% of the European GMP NAC formulations, which are intended for oral administration and have qualified for health insurance reimbursement[207].

Several US nutraceutical dealers manufacture and sell unbuffered (acidic) NAC. Since the production and packaging of nutraceutical products in the US is not regulated by the FDA, neither the content nor the purity of the NAC formulations currently produced and marketed in the US can be reliably judged. Manufacturing methods for these NAC preparations may not prevent formation of NAC by-products (e.g., di-NAC) and may not have been validated for stability during storage.

We (LAH, JPA, SCD) have recently suggested[262, 263] that NAC be administered, and perhaps co-formulated, with acetaminophen. Animal studies suggest that administration of roughly equimolar amounts of NAC and acetaminophen might be sufficient to accomplish this goal[264]. Co-administration of NAC with a GSH-depleting drug such as acetaminophen could decrease the toxicity of the drug, particular in settings where cysteine/GSH deficiency is likely. Thus, there is strong reason to believe that co-administered NAC would more safely allow administration of acetaminophen or other GSH-depleting drugs when clinically warranted.

### Conclusion: Cysteine/GSH Deficiency—a New Clinical Entity

The evidence reviewed here reveals cysteine/GSH deficiency as an emerging clinical entity. The manifestations of this deficiency may vary in different disease settings, as may the biochemical mechanisms that mediate its effects. However, they are united by a common positive response to NAC therapy in over fifty randomized placebo-controlled trials (see table 1). Thus, the studies we have reviewed collectively argue for consideration of cysteine/GSH deficiency as a significant and treatable clinical entity.

Surprisingly, given the diverse roles that GSH plays in cellular physiology and regulation of enzyme activity and protein function, the consequences of low GSH levels have mainly been discussed from a clinical perspective in terms of the loss of protection against intracellular oxidative stress. However, while antioxidants such as vitamin E and vitamin C can spare GSH under conditions of oxidative stress, GSH loss can only be offset by GSH resynthesis, indicating a central role for this molecule over and above its ability to counteract the effects of intracellular oxidants.

Similarly, although NAC is a well-known source of cysteine for GSH replenishment in acetaminophen toxicity, it is principally cast as an antioxidant in other settings. By and large, physicians and the public in general tend to equate NAC with vitamin C, vitamin E and other antioxidants. Indeed, like GSH, NAC can itself serve as an antioxidant. However, while other antioxidants can replace NAC and GSH in this role, only NAC or another cysteine source can provide the raw material necessary to replenish GSH and to enable GSH-dependent biochemical reactions.

We have pointed out that physicians may find NAC administration useful as adjunct therapy for diseases and conditions in which cysteine/GSH deficiency is likely to play a role. The positive findings in the placebo-controlled studies we have discussed support this argument. However, the absence of large multicenter trials testing NAC in various settings leaves this evidence still open to question. Hopefully, the recognition that cysteine/GSH deficiency is an important clinical entity will encourage support for such trials.

In the meantime, a conservative approach to the findings we have discussed suggests that patients with diseases or conditions in which cysteine/GSH deficiency has been demonstrated may be well advised to avoid unnecessary behaviors and exposures to medications that may exacerbate GSH depletion. In fact, when advising such patients, it seems reasonable for physicians to emphasize that alcohol usage be kept at modest levels and that acetaminophen usage should be kept strictly within the recommended dosing (or perhaps be accompanied by NAC administration).

The availability of over-the-counter NAC, and the low toxicity of this cysteine prodrug in situations where it has been tested, opens the possibility of patient or physician initiated therapy. However, if such therapy is elected, we suggest that the NAC preparation(s) used be prepared under GMP conditions and packaged to prevent oxidation of the product.

### DETAILED DESCRIPTION OF THE INVENTION

The invention described herein provides a method of treatment to prevent development of gluathione deficiency as a consequence of disease, a treatment, or a condition comprising administering to a subject at risk of glutathione deficieny as a consequence of disease, a treatment or a condition a therapeutic amount of NAC or a pharmaceutically acceptable salt or derivative sufficient to increase intracellular glutathione levels or decrease oxidative stress and measuring and monitoring the level of glutathione in blood in patients as needed. It further provides a method of treatment to restore glutathione levels comprising administering to subjects in need of glutathone level restoration, as determined by measurement or by a physician, a therapeutic amount of NAC or a pharmaceutically acceptable salt or derivative thereof sufficient to increase intracellular glutathione levels or decrease oxidative stress and monitoring restoration by measuring the level of glutathione in blood as needed. It futher provides a method of treatment to decrease oxidized glutathione levels elevated as a consequence of disease, a treatment or a condition comprising administering to a subject suffering from oxidative stress a therapeutic amount of NAC or a pharmaceutically acceptable salt or derivative sufficient to decrease oxidized glutathione levels elevated as a consequence of disease and monitoring the level of oxidized glutathione in blood as needed.

The term “pharmaceutical composition” is used herein to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

The term “therapeutically effective amount” is used herein to denote that amount of a drug or pharmaceutical agent that will elicit the therapeutic response of an animal or human that is being sought.

The pharmaceutical compositions containing NAC may be in a form suitable for oral use, for example, as tablets, troches, lozenges, aqueous, or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain NAC in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, to form osmotic therapeutic tablets for controlled release.

Formulations for oral use may also be presented as hard gelatin capsules where NAC is mixed with an inert solid diluent, for example, calcium carbonate, calcium phosphate or kaolin, or a soft gelatin capsules wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents, one or more flavoring agents, and one or more sweetening agents, such as sucrose or saccharin.

Oily suspensions may be formulated by suspending NAC in a vegetable oil, for example arachis oil, olive oil, sesame oil or coconut oil, or in a mineral oil such as a liquid paraffin. The oily suspensions may contain a thickening agent, for example beeswax, hard paraffin or cetyl alchol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide NAC in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing NAC alone or in combination with a therapeutically effective amount of a therapeutic agent with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The NAC alone or in combination with a therapeutically effective amount of a therapeutic agent may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

Pharmaceutically-acceptable salts of NAC alone or in combination with a therapeutically effective amount of a therapeutic agent, where a basic or acidic group is present in the structure, are also included within the scope of the invention. The term “pharmaceutically acceptable salts” refers to non-toxic salts of NAC alone or in combination with a therapeutically effective amount of a therapeutic agent, which are generally prepared by reacting the free base with a suitable organic or inorganic acid or by reacting the acid with a suitable organic or inorganic base. Representative salts include the following salts: Acetate, Benzenesulfonate, Benzoate, Bicarbonate, Bisulfate, Bitartrate, Borate, Bromide, Calcium Edetate, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrocloride, Hydroxynaphthoate, Iodide, Isethionate, Lactate, Lactobionate, Laurate, Malate, Maleate, Mandelate, Methanesulfonate, Methylbromide, Methylnitrate, Methylsulfate, Monopotassium Maleate, Mucate, Napsylate, Nitrate, N-methylglucamine, Oxalate, Pamoate (Embonate), Palmitate, Pantothenate, Phosphate/diphosphate, Polygalacturonate, Potassium, Salicylate, Sodium, Stearate, Subacetate, Succinate, Tannate, Tartrate, Teoclate, Tosylate, Triethiodide, Trimethylammonium and Valerate. When an acidic substituent is present, such as-COOH, there can be formed the ammonium, morpholinium, sodium, potassium, barium, calcium salt, and the like, for use as the dosage form. When a basic group is present, such as amino or a basic heteroaryl radical, such as pyridyl, an acidic salt, such as hydrochloride, hydrobromide, phosphate, sulfate, trifluoroacetate, trichloroacetate, acetate, oxlate, maleate, pyruvate, malonate, succinate, citrate, tartarate, fumarate, mandelate, benzoate, cinnamate, methanesulfonate, ethanesulfonate, picrate and the like, and include acids related to the pharmaceutically-acceptable salts listed in the Journal of Pharmaceutical Science, 66, 2 (I 977) p. 1-19.

Other salts which are not pharmaceutically acceptable may be useful in the preparation of NAC alone or in combination with a therapeutically effective amount of a therapeutic agent and these form a further aspect of the invention.

Thus, in another aspect of the present invention, there is provided a pharmaceutical composition comprising NAC alone or in combination with a therapeutically effective amount of a therapeutic agent, or a pharmaceutically acceptable salt, solvate, or prodrug therof, and one or more pharmaceutically acceptable carriers, excipients, or diluents.

In another aspect of the present invention, there is provided a pharmaceutical composition comprising a therapeutically effective amount of NAC alone or in combination with a therapeutically effective amount of a therapeutic agent, or a pharmaceutically acceptable salt, solvate, or prodrug therof, and one or more pharmaceutically acceptable carriers, excipients, or diluents, wherein said therapeutically effective amount comprises a sufficient amount NAC for at least partial amelioration of cysteine/glutathione deficiency in a disease or condition. In an embodiment of the pharmaceutical composition, the disease comprises chronic obstructive pulmonary disease (COPD). In another embodiment of the pharmaceutical composition, the disease comprises acute renal failure. In another embodiment of the pharmaceutical composition, the disease comprises sickle cell anemia. In another embodiment of the pharmaceutical composition, the disease comprises comprises diabetes mellitus. In another embodiment of the pharmaceutical composition, the disease comprises inflammatory diseases. In another embodiment of the pharmaceutical composition, the disease comprises human immunodeficiency virus mediated disease. In another embodiment of the pharmaceutical composition, the disease comprises malaria. In another embodiment of the pharmaceutical composition, the disease comprises protein-energy malnutrition. In another embodiment of the pharmaceutical composition, the disease comprises otic disease. In another embodiment of the pharmaceutical composition, the disease comprises neurodegenerative disease. In another embodiment of the pharmaceutical composition, the disease comprises cardiovascular disease.

In another aspect, the present invention provides a pharmaceutical composition comprising a therapeutically effective amount of NAC alone or in combination with a therapeutically effective amount of a therapeutic agent, and one or more pharmaceutically acceptable carriers, excipients, or diluents, wherein the pharmaceutical composition is used to replace or supplement compounds that restore cysteine/glutathione levels.

In another aspect, the present invention provides a method for improvement of cysteine/glutathione deficiency in a disease or condition comprising administering to a subject in need thereof a NAC alone or in combination with a therapeutically effective amount of a therapeutic agent, wherein NAC alone or in combination with a therapeutically effective amount of a therapeutic agent is administered to the subject as a pharmaceutical composition comprising a therapeutically effective amount of NAC and one or more pharmaceutically acceptable carriers, excipients, or diluents, wherein said therapeutically effective amount of NAC comprises a sufficient amount of NAC for treatment or prevention of cysteine-glutathione deficiency in the disease or condition.

The term “treatment” as used herein, refers to the full spectrum of treatments for a given disorder from which the patient is suffering, including alleviation of one, most of all symptoms resulting from that disorder, to an outright cure for the particular disorder or prevention of the onset of the disorder.

Generally speaking, the amount of NAC per dosage unit is preferably from 1 mg to 25000 mg, preferably at least 3 mg to 2000 mg per dosage unit for oral administration, and 20-20000 mg for parenteral administration. The unit dose of NAC alone or in combination with a therapeutically effective amount of a therapeutic agent, will usually comprise at least about 1.5 mg/kg to a maximum amount of 70 mg/kg (for pediatric doses), usually at least about 500 mg (for adult doses), and usually not more than about 2000 mg at the physician's discretion. Patients on therapy known to deplete cysteine/glutathione or produce oxidative stress may benefit from higher amounts of NAC. The amount of active ingredient that may be combined with the carrier materials to produce a single dosage will vary depending upon the host treated and the particular mode of administration.

This dosage has to be individualized by the clinician based on the specific clinical condition of the subject being treated. Thus, it will be understood that the specific dosage level for any particular patient will depend upon a variety of factors including the activity of the specific compound employed, the age, body weight, general health, sex, diet, time of administration, route of administration, rate of excretion, drug combination and the severity of the particular disease undergoing therapy.

While the invention has been described and illustrated with reference to certain preferred embodiments therof, those skilled in the art will appreciate that various changes, modifications and substitutions can be made therein without departing from the spirit and scope of the invention. For example, effective dosages other than the preferred dosages as set forth herein may be applicable as a consequence of variations in the responsiveness of the mammal being treated for cysteine/glutathione deficiency in a disease or condition. Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention.

### CITATIONS

1. Meister A, Anderson M E: Glutathione. Annu Rev Biochem 1983, 52:711-760.
2. Keays R, Harrison P M, Wendon J A, Forbes A, Gove C, Alexander G J, Williams R: Intravenous acetylcysteine in paracetamol induced fulminant hepatic failure: a prospective controlled trial. Bmj 1991, 303(6809):1026-1029.
3. Tepel M, van der Giet M, Schwarzfeld C, Laufer U, Liermann D, Zidek W: Prevention of radiographic-contrast-agent-induced reductions in renal function by acetylcysteine. N Engl J Med 2000, 343((20 July)): 180-184.
4. Tepel M, Zidek W: Acetylcysteine for radiocontrast nephropathy. Curr Opin Crit Care 2001, 7(6):390-392.
5. Tepel M: Acetylcysteine for the prevention of radiocontrast-induced nephropathy. Minerva Cardioangiol 2003, 51(5):525-530.
6. Kay J, Chow W H, Chan T M, Lo S K, Kwok O H, Yip A, Fan K, Lee C H, Lam W F: Acetylcysteine for prevention of acute deterioration of renal function following elective coronary angiography and intervention: a randomized controlled trial. Jama 2003, 289(5):553-558.
7. Diaz-Sandoval L J, Kosowsky B D, Losordo D W: Acetylcysteine to prevent angiography-related renal tissue injury (the APART trial). Am J Cardiol 2002, 89(3):356-358.
8. Shyu K G, Cheng J J, Kuan P: Acetylcysteine protects against acute renal damage in patients with abnormal renal function undergoing a coronary procedure. J Am Coll Cardiol 2002, 40(8):1383-1388.
9. Efrati S, Dishy V, Averbukh M, Blatt A, Krakover R, Weisgarten J, Morrow J D, Stein M C, Golik A: The effect of N-acetylcysteine on renal function, nitric oxide, and oxidative stress after angiography. Kidney Int 2003, 64(6):2182-2187.
10. MacNeill B D, Harding S A, Bazari H, Patton K K, Colon-Hemadez P, DeJoseph D, Jang I K: Prophylaxis of contrast-induced nephropathy in patients undergoing coronary angiography. Catheter Cardiovasc Interv 2003, 60(4):458-461.
11. Spies C, Giese C, Meier-Hellmann A, Specht M, Hannemann L, Schaffartzik W, Reinhart K: [The effect of prophylactically administered n-acetylcysteine on clinical indicators for tissue oxygenation during hyperoxic ventilation in cardiac risk patients]. Anaesthesist 1996, 45(4):343-350.
12. Svendsen J H, Klarlund K, Aldershvile J, Waldorff S: N-acetylcysteine modifies the acute effects of isosorbide-5-mononitrate in angina pectoris patients evaluated by exercise testing. J Cardiovasc Pharmacol 1989, 13(2):320-323.
13. Boesgaard S, Aldershvile J, Pedersen F, Pietersen A, Madsen J K, Grande P: Continuous oral N-acetylcysteine treatment and development of nitrate tolerance in patients with stable angina pectoris. J Cardiovasc Pharmacol 1991, 17(6):889-893.
14. Boesgaard S, Aldershvile J, Poulsen H E: Preventive administration of intravenous N-acetylcysteine and development of tolerance to isosorbide dinitrate in patients with angina pectoris. Circulation 1992, 85(1):143-149.
15. Vita J A, Frei B, Holbrook M, Gokce N, Leaf C, Keaney Jr J F: L-2-oxothiazolidine-4-carboxylic acid reverses endothelial dysfunction in patients with coronary artery disease. J Clin Invest 1998, 101(6):1408-1414.
16. Andersen L W, Thiis J, Kharazmi A, Rygg I: The role of N-acetylcystein administration on the oxidative response of neutrophils during cardiopulmonary bypass. Perfusion 1995, 10(1):21-26.
17. Tepel M, van der Giet M, Statz M, Jankowski J, Zidek W: The Antioxidant Acetylcysteine Reduces Cardiovascular Events in Patients With End-Stage Renal Failure. A Randomized, Controlled Trial. Circulation 2003, 107:992-995.
18. Pace B S, Shartava A, Pack-Mabien A, Mulekar M, Ardia A, Goodman S R: Effects of N-acetylcysteine on dense cell formation in sickle cell disease. Am J Hematol 2003, 73(1):26-32.
19. Bromley P N, Cottam S J, Hilmi I, Tan K C, Heaton N, Ginsburg R, Potter D R: Effects of intraoperative N-acetylcysteine in orthotopic liver transplantation. Br J Anaesth 1995, 75(3):352-354.
20. Fulghesu A M, Ciampelli M, Muzj G, Belosi C, Selvaggi L, Ayala G F, Lanzone A: N-acetyl-cysteine treatment improves insulin sensitivity in women with polycystic ovary syndrome. Fertil Steril 2002, 77(6):1128-1135.
21. De Mattia G, Bravi M C, Laurenti O, Cassone-Faldetta M, Proietti A, De Luca O, Armiento A, Ferri C: Reduction of oxidative stress by oral N-acetyl-L-cysteine treatment decreases plasma soluble vascular cell adhesion molecule-1 concentrations in non-obese, non-dyslipidaemic, normotensive, patients with non-insulin-dependent diabetes. Diabetologia 1998, 41(11):1392-1396.
22. De Mattia G, Bravi M C, Laurenti O, Cassone-Faldetta M, Armiento A, Ferri C, Balsano F: Influence of reduced glutathione infusion on glucose metabolism in patients with non-insulin-dependent diabetes mellitus. Metabolism 1998, 47(8):993-997.
23. Bylin G, Hedenstiema G, Lagerstrand L, Wagner P D: No influence of acetylcysteine on gas exchange and spirometry in chronic asthma. Eur J Respir Dis 1987, 71(2):102-107.
24. Grassi C, Morandini G C: A controlled trial of intermittent oral acetylcysteine in the long-term treatment of chronic bronchitis. Eur J Clin Pharmacol 1976, 09(5-6):393-396.
25. Aylward M, Maddock J, Dewland P: Clinical evaluation of acetylcysteine in the treatment of patients with chronic obstructive bronchitis: a balanced double-blind trial with placebo control. Eur J Respir Dis Suppl 1980, 111:81-89.
26. Brocard H, Charpin J, Germouty J: [Multicenter, double-blind study of oral acetylcysteine vs. placebo]. Eur J Respir Dis Suppl 1980, 111:65-69.
27. Grassi C: Long-term oral acetylcysteine in chronic bronchitis. a double-blind controlled study. Eur J Respir Dis Suppl 1980, 111:93-108.
28. Ferrari V, Spinelli W: Life table analysis of long-term randomised trials in pneumology—a worked example and a plea. Eur J Respir Dis Suppl 1980, 110:227-236.
29. Boman G, Backer U, Larsson S, Melander B, Wahlander L: Oral acetylcysteine reduces exacerbation rate in chronic bronchitis: report of a trial organized by the Swedish Society for Pulmonary Diseases. Eur J Respir Dis 1983, 64(6):405-415.
30. Jackson I M, Barnes J, Cooksey P: Efficacy and tolerability of oral acetylcysteine (Fabrol) in chronic bronchitis: a double-blind placebo controlled study. J Int Med Res 1984, 12(3): 198-206.
31. Parr G D, Huitson A: Oral Fabrol (oral N-acetyl-cysteine) in chronic bronchitis. Br J Dis Chest 1987, 81(4):341-348.
32. Rasmussen J B, Glennow C: Reduction in days of illness after long-term treatment with N-acetylcysteine controlled-release tablets in patients with chronic bronchitis. Eur Respir J 1988, 1(4):351-355.
33. Evald T, Hansen M, Balslov S, Brorson-Riis L, Hansen N C, Maltbaek N, Thorshauge H: [Steroid response after long-term treatment with oral N-acetylcysteine in patients with chronic obstructive bronchitis]. Ugeskr Laeger 1989, 151(46):2076-2078.
34. Hansen N C, Skriver A, Brorsen-Riis L, Balslov S, Evald T, Maltbaek N, Gunnersen G, Garsdal P, Sander P, Pedersen J Z et al: Orally administered N-acetylcysteine may improve general well-being in patients with mild chronic bronchitis. Respir Med 1994, 88(7):531-535.
35. Van Schooten F J, Nia A B, De Flora S, D'Agostini F, Izzotti A, Camoirano A, Balm A J, Dallinga J W, Bast A, Haenen G R et al: Effects of oral administration of N-acetyl-L-cysteine: a multi-biomarker study in smokers. Cancer Epidemiol Biomarkers Prev 2002, 11(2):167-175.
36. Olivieri D, Marsico S A, Del Donno M: Improvement of mucociliary transport in smokers by mucolytics. Eur J Respir Dis Suppl 1985, 139:142-145.
37. Travaline J M, Sudarshan S, Roy B G, Cordova F, Leyenson V, Criner G J: Effect of N-acetylcysteine on human diaphragm strength and fatigability. Amer J Respir Crit Care Med 1997, 156(5):1567-1571.
38. Todisco T, Polidori R, Rossi F, Iannacci L, Bruni B, Fedeli L, Palumbo R: Effect of N-acetylcysteine in subjects with slow pulmonary mucociliary clearance. Eur J Respir Dis Suppl 1985, 139:136-141.
39. Verstraeten J M: Mucolytic treatment in chronic obstructive pulmonary disease. Double-blind comparative clinical trial with N-acetylcysteine, bromhexine and placebo. Acta Tuberc Pneumol Belg 1979, 70(1):71-80.
40. Stafanger G, Game S, Howitz P, Morkassel E, Koch C: The clinical effect and the effect on the ciliary motility of oral N-acetylcysteine in patients with cystic fibrosis and primary ciliary dyskinesia. Eur Respir J 1988, 1(2): 161-167.
41. Stafanger G, Koch C: N-acetylcysteine in cystic fibrosis and Pseudomonas aeruginosa infection: clinical score, spirometry and ciliary motility. Eur Respir J 1989, 2(3):234-237.
42. Spies C D, Reinhart K, Witt I, Meier-Hellmann A, Hannemann L, Bredle D L, Schaffartzik W: Influence of N-acetylcysteine on indirect indicators of tissue oxygenation in septic shock patients: results from a prospective, randomized, double-blind study. Crit Care Med 1994, 22(11):1738-1746.
43. Spapen H, Zhang H, Demanet C, Vleminckx W, Vincent J L, Huyghens L: Does N-acetyl-L-cysteine influence cytokine response during early human septic shock? Chest 1998, 113(6):1616-1624.
44. Rank N, Michel C, Haertel C, Lenhart A, Welte M, Meier-Hellmann A, Spies C: N-acetylcysteine increases liver blood flow and improves liver function in septic shock patients: results of a prospective, randomized, double-blind study. Crit Care Med 2000, 28(12):3799-3807.
45. Agusti A G, Togores B, Ibanez J, Raurich J M, Maimo A, Bergada J, Marse P, Jorda R: Effects of N-acetylcysteine on tissue oxygenation in patients with multiple organ failure and evidence of tissue hypoxia. Eur Respir J 1997, 10(9):1962-1966.
46. De Flora S, Grassi C, Carati L: Attenuation of influenza-like symptomatology and improvement of cell-mediated immunity with long-term N-acetylcysteine treatment. Eur Respir J 1997, 10(7):1535-1541.
47. Akerlund B, Jarstrand C, Lindeke B, Sonnerborg A, Akerblad A-C, Rasool O: Effect of N-acetylcysteine (NAC) treatment on HIV-1 infection: a double-blind placebo-controlled trial. Eur J Clin Pharmacol 1996, 50(6):457-461.
48. De Rosa S C, Zaretsky M D, Dubs J G, Roederer M, Anderson M, Green A, Mitra D, Watanabe N, Nakamura H, Tjioe I et al: N-acetylcysteine replenishes glutathione in HIV infection. Eur J Clin Invest 2000, 30(10):915-929.
49. Herzenberg L A, De Rosa S C, Dubs J G, Roederer M, Anderson M T, Ela S W, Deresinski S C: Glutathione deficiency is associated with impaired survival in HIV disease. Proc Natl Acad Sci USA 1997, 94(5):1967-1972.
50. Breitkreutz R, Pittack N, Nebe C T, Schuster D, Brust J, Beichert M, Hack V, Daniel V, Edler L, Droge W: Improvement of immune functions in HIV infection by sulfur supplementation: two randomized trials. J Mol Med 2000, 78(1):55-62.
51. Watt G, Jongsakul K, Ruangvirayuth R: A pilot study of N-acetylcysteine as adjunctive therapy for severe malaria. Qim 2002, 95(5):285-290.
52. Treeprasertsuk S, Krudsood S, Tosukhowong T, Maek A N W, Vannaphan S, Saengnetswang T, Looareesuwan S, Kuhn W F, Brittenham G, Carroll J: N-acetylcysteine in severe falciparum malaria in Thailand. Southeast Asian J Trop Med Public Health 2003, 34(1):37-42.
53. Ovesen T, Felding J U, Tommerup B, Schousboe L P, Petersen C G: Effect of N-acetylcysteine on the incidence of recurrence of otitis media with effusion and re-insertion of ventilation tubes. Acta Oto—Laryngol 2000:79-81.
54. Badaloo A, Reid M, Forrester T, Heird W C, Jahoor F: Cysteine supplementation improves the erythrocyte glutathione synthesis rate in children with severe edematous malnutrition. Am J Clin Nutr 2002, 76(3):646-652.
55. Estensen R D, Levy M, Klopp S J, Galbraith A R, Mandel J S, Blomquist J A, Wattenberg L W: N-acetylcysteine suppression of the proliferative index in the colon of patients with previous adenomatous colonic polyps. 1999, 147(1-2): 109-114.
56. Walters M T, Rubin C E, Keightley S J, Ward C D, Cawley M I: A double-blind, cross-over, study of oral N-acetylcysteine in Sjogren's syndrome. Scand J Rheumatol Suppl 1986, 61:253-258.
57. Yalcin E, Altin F, Cinhuseyinoglue F, Arslan M O: N-acetylcysteine in chronic blepharitis. Cornea 2002, 21(2): 164-168.
58. Medved I, Brown M J, Bjorksten A R, Leppik J A, Sostaric S, McKenna M J: N-acetylcysteine infusion alters blood redox status but not time to fatigue during intense exercise in humans. J Appl Physiol 2003, 94(4): 1572-1582.
59. Hauer K, Hildebrandt W, Sehl Y, Edler L, Oster P, Droge W: Improvement in muscular performance and decrease in tumor necrosis factor level in old age after antioxidant treatment. J Mol Med 2003, 81(2): 118-125.
60. Adair J C, Knoefel J E, Morgan N: Controlled trial of N-acetylcysteine for patients with probable Alzheimer's disease. Neurology 2001, 57(8): 1515-1517.
61. Bowles W H, Goral V: Clinical trial of the anti-plaque activity of a mucolytic agent, N-Acetyl Cysteine. Dent Hyg (Chic) 1985, 59(10):454-456.
62. Marini U, Visconti G, Spotti D, Geniram A: Controlled endoscopic study on gastroduodenal safety of acetylcysteine after oral administration. Eur J Respir Dis Suppl 1980, 111:147-150.
63. Abrams D, Cotton D, Mayer K: AIDS/HIV Treatment Directory, vol. 7 #4. Rockville, Md.: American Foundation for AIDS Research (AmFAR); 1995.
64. Hortin G L, Landt M, Powderly W G: Changes in plasma amino acid concentrations in response to HIV-1 infection. Clin Chem 1994, 40(5):785-789.
65. Breitkreutz R, Holm S, Pittack N, Beichert M, Babylon A, Yodoi J, Droge W: Massive loss of sulfur in HIV infection. AIDS Res Hum Retroviruses 2000, 16(3):203-209.
66. Evaluation of Protein Nutrition. Bethesda, Md.: National Academy Press; 1989.
67. Hitchins A D, McDonough F E, Wells P A: The use of Escherichia coli mutants to measure the bioavailability of essential amino acids in foods. Plant Foods Hum Nutr 1989, 39(1):109-120.
68. Volkin D B, Klibanov A M: Thermal destruction processes in proteins involving cystine residues. J Biol Chem 1987, 262(7):2945-2950.
69. Schnack U, Klostermeyer H: Thermischer Abbau von—Lactalbumin. Milchwissenschaft 1980, 35(4):206-208.
70. Scharf U, Weder J: Model studies on the heating of food proteins. Chem MikrobiolTechnol Lebensm 1983, 8:71-77.
71. Larsen L C, Fuller S H: Management of acetaminophen toxicity. Am Fam Physician 1996, 53(1):185-190.
72. Shriner K, Goetz M B: Severe hepatotoxicity in a patient receiving both acetaminophen and zidovudine. Am J Med 1992, 93(1):94-96.
73. Holmgren A: Thioredoxin and glutaredoxin systems. J Biol Chem 1989, 264(24): 13963-13966.
74. Holmgren A: Antioxidant function of thioredoxin and glutaredoxin systems. Antioxid Redox Signal 2000, 2(4):811-820.
75. Amer E S, Holmgren A: Physiological functions of thioredoxin and thioredoxin reductase. Eur J Biochem 2000, 267(20):6102-6109.
76. Kanner S B, Kavanagh T J, Grossmann A, Hu S L, Bolen J B, Rabinovitch P S, Ledbetter J A: Sulfhydryl oxidation down-regulates T-cell signaling and inhibits tyrosine phosphorylation of phospholipase C gamma 1. Proc Natl Acad Sci USA 1992, 89(1):300-304.
77. Flescher E, Ledbetter J A, Schieven G L, Velaroch N, Fossum D, Dang H, Ogawa N, Talal N: Longitudinal exposure of human T lymphocytes to weak oxidative stress suppresses transmembrane and nuclear signal transduction. Journal of Immunology 1994, 153(11):4880-4889.
78. Ahmad M, Zhang Y, Papharalambus C, Alexander R W: Role of isoprenylcysteine carboxylmethyltransferase in tumor necrosis factor-alpha stimulation of expression of vascular cell adhesion molecule-1 in endothelial cells. Arterioscler Thromb Vasc Biol 2002, 22(5):759-764.
79. Marui N, Offermann M K, Swerlick R, Kunsch C, Rosen C A, Ahmad M, Alexander R W, Medford R M: Vascular cell adhesion molecule-1 (VCAM-1) gene transcription and expression are regulated through an antioxidant-sensitive mechanism in human vascular endothelial cells. J Clin Invest 1993, 92(4): 1866-1874.
80. Schmidt A M, Hori O, Chen J X, Li J F, Crandall J, Zhang J, Cao R, Yan S D, Brett J, Stern D: Advanced glycation endproducts interacting with their endothelial receptor induce expression of vascular cell adhesion molecule-1 (VCAM-1) in cultured human endothelial cells and in mice. A potential mechanism for the accelerated vasculopathy of diabetes. J Clin Invest 1995, 96(3):1395-1403.
81. Weigand M A, Plachky J, Thies J C, Spies-Martin D, Otto G, Martin E, Bardenheuer H J: N-acetylcysteine attenuates the increase in alpha-glutathione S-transferase and circulating ICAM-1 and VCAM-1 after reperfusion in humans undergoing liver transplantation. Transplantation 2001, 72(4):694-698.
82. Ghezzi P, Romines B, Fratelli M, Eberini 1, Gianazza E, Casagrande S, Laragione T, Mengozzi M, Herzenberg L A: Protein glutathionylation: coupling and uncoupling of glutathione to protein thiol groups in lymphocytes under oxidative stress and HIV infection. Mol Immunol 2002, 38(10):773-780.
83. Wang J, Boja E S, Tan W, Tekle E, Fales H M, English S, Mieyal J J, Chock P B: Reversible glutathionylation regulates actin polymerization in A431 cells. J Biol Chem 2001, 276(51):47763-47766.
84. Pineda-Molina E, Klatt P, Vazquez J, Marina A, Garcia de Lacoba M, Perez-Sala D, Lamas S: Glutathionylation of the p50 subunit of NF-kappaB: a mechanism for redox-induced inhibition of DNA binding. Biochemistry 2001, 40(47):14134-14142.
85. Galli F, Rossi R, Di Simplicio P, Floridi A, Canestrari F: Protein thiols and glutathione influence the nitric oxide-dependent regulation of the red blood cell metabolism. Nitric Oxide 2002, 6(2):186-199.
86. Choudhary G, Dudley S C, Jr.: Heart failure, oxidative stress, and ion channel modulation. Congest Heart Fail 2002, 8(3):148-155.
87. Estevez A G, Jordan J: Nitric oxide and superoxide, a deadly cocktail. Ann NY Acad Sci 2002, 962:207-211.
88. Yang E S, Richter C, Chun J S, Huh T L, Kang S S, Park J W: Inactivation of NADP(+)-dependent isocitrate dehydrogenase by nitric oxide. Free Radic Biol Med 2002, 33(7):927-937.
89. Amelle D R, Stamler J S: NO+, NO, and NO− donation by S-nitrosothiols: implications for regulation of physiological functions by S-nitrosylation and acceleration of disulfide formation. Arch Biochem Biophys 1995, 318(2):279-285.
90. Gow A J, Chen Q, Hess D T, Day B J, Ischiropoulos H, Stamler J S: Basal and stimulated protein S-nitrosylation in multiple cell types and tissues. J Biol Chem 2002, 277(12):9637-9640.
91. Marshall H E, Stamler J S: Inhibition of NF-kappa B by S-nitrosylation. Biochemistry 2001, 40(6):1688-1693.
92. Marshall H E, Stamler J S: Nitrosative stress-induced apoptosis through inhibition of NF-kappa B. J Biol Chem 2002, 277(37):34223-34228.
93. Cotgreave I A, Gerdes R, Schuppe-Koistinen I, Lind C: S-glutathionylation of glyceraldehyde-3-phosphate dehydrogenase: role of thiol oxidation and catalysis by glutaredoxin. Methods Enzymol 2002, 348:175-182.
94. Nakamura H, De Rosa S C, Yodoi J, Holmgren A, Ghezzi P, Herzenberg L A: Chronic elevation of plasma thioredoxin: Inhibition of chemotaxis and curtailment of life expectancy in AIDS. Proc Natl Acad Sci USA 2001, 98(5):2688-2693.
95. Vlamis-Gardikas A, Holmgren A: Thioredoxin and glutaredoxin isoforms. Methods Enzymol 2002, 347:286-296.
96. Casagrande S, Bonetto V, Fratelli M, Gianazza E, Eberini 1, Massignan T, Salmona M, Chang G, Holmgren A, Ghezzi P: Glutathionylation of human thioredoxin: a possible crosstalk between the glutathione and thioredoxin systems. Proc Natl Acad Sci USA 2002, 99(15):9745-9749.
97. Daily D, Vlamis-Gardikas A, Offen D, Mittelman L, Melamed E, Holmgren A, Barzilai A: Glutaredoxin protects cerebellar granule neurons from dopamine-induced apoptosis by activating NF-kappa B via Ref-1. J Biol Chem 2001, 276(2): 1335-1344.
98. Shenton D, Perrone G, Quinn K A, Dawes I W, Grant C M: Regulation of protein S-thiolation by glutaredoxin 5 in the yeast Saccharomyces cerevisiae. J Biol Chem 2002, 277(19):16853-16859.
99. Song J J, Rhee J G, Suntharalingam M, Walsh S A, Spitz D R, Lee Y J: Role of glutaredoxin in metabolic oxidative stress: Glutaredoxin as a sensor of oxidative stress mediated by H₂O₂. J Biol Chem 2002.
100. Saitoh M, Nishitoh H, Fujii M, Takeda K, Tobiume K, Sawada Y, Kawabata M, Miyazono K, Ichijo H: Mammalian thioredoxin is a direct inhibitor of apoptosis signal-regulating kinase (ASK) 1. Embo J 1998, 17(9):2596-2606.
101. Gladyshev V N, Jeang K T, Stadtman T C: Selenocysteine, identified as the penultimate C-terminal residue in human T-cell thioredoxin reductase, corresponds to TGA in the human placental gene. Proc Natl Acad Sci USA 1996, 93(12):6146-6151.
102. Gladyshev V N, Khangulov S V, Stadtman T C: Properties of the selenium- and molybdenum-containing nicotinic acid hydroxylase from Clostridium barkeri. Biochemistry 1996, 35(1):212-223.
103. Sandalova T, Zhong L, Lindqvist Y, Holmgren A, Schneider G: Three-dimensional structure of a mammalian thioredoxin reductase: implications for mechanism and evolution of a selenocysteine-dependent enzyme. Proc Natl Acad Sci USA 2001, 98(17):9533-9538.
104. Zhong L, Amer E S, Holmgren A: Structure and mechanism of mammalian thioredoxin reductase: the active site is a redox-active selenolthiol/selenenylsulfide formed from the conserved cysteine-selenocysteine sequence. Proc Natl Acad Sci USA 2000, 97(11):5854-5859.
105. Zhong L, Holmgren A: Essential role of selenium in the catalytic activities of mammalian thioredoxin reductase revealed by characterization of recombinant enzymes with selenocysteine mutations. J Biol Chem 2000, 275(24):18121-18128.
106. Aaseth J, Stoa-Birketvedt G: Glutathione in overweight patients with poorly controlled type 2 diabetes. Journal of Trace Elements in Experimental Medicine 2000, 13(1):105-111.
107. Meister A, Anderson M E, Hwang O: Intracellular cysteine and glutathione delivery systems. J Am Coll Nutr 1986, 5(2):137-151.
108. Castagna A, Legrazie C, Accordini A, Giulidori P, Cavalli G, Bottiglieri T, Lazzarin A: Cerebrospinal fluid S-adenosylmethionine (SAMe) and glutathione concentrations in HIV infection: effect of parenteral treatment with SAMe. Neurology 1995, 45(9):1678-1683.
109. Mitchell J R, Thorgeirsson S S, Potter W Z, Jollow D J, Keiser H: Acetaminophen-induced hepatic injury: protective role of glutathione in man and rationale for therapy. Clin Pharmacol Ther 1974, 16(4):676-684.
110. Peterson R G, Rumack B H: Toxicity of acetaminophen overdose. Jacep 1978, 7(5):202-205.
111. Mitchell J R, Corcoran G B, Smith C V, Hughes H, Lauterburg B H: Alkylation and peroxidation injury from chemically reactive metabolites. Adv Exp Med Biol 1981, 136(Pt A): 199-223.
112. Lauterburg B H, Corcoran G B, Mitchell J R: Mechanism of action of N-acetylcysteine in the protection against the hepatotoxicity of acetaminophen in rats in vivo. J Clin Invest 1983, 71(4):980-991.
113. Smilkstein M J, Knapp G L, Kulig K W, Rumack B H: Efficacy of oral N-acetylcysteine in the treatment of acetaminophen overdose. Analysis of the national multicenter study (1976 to 1985). N Engl J Med 1988, 319(24):1557-1562.
114. Ostapowicz G, Fontana R J, Schiodt F V, Larson A, Davern T J, Han S H, McCashland T M, Shakil A O, Hay J E, Hynan L et al: Results of a prospective study of acute liver failure at 17 tertiary care centers in the United States. Ann Intern Med 2002, 137(12):947-954.
115. Ostapowicz G, Lee W M: Acute hepatic failure: a Western perspective. J Gastroenterol Hepatol 2000, 15(5):480-488.
116. Lyons L, Studdiford J S, Sommaripa A M: Treatment of acetaminophen overdosage with N-acetylcysteine. N Engl J Med 1977, 296(3):174-175.
117. Prescott L F, Park J, Ballantyne A, Adriaenssens P, Proudfoot A T: Treatment of paracetamol (acetaminophen) poisoning with N− acetylcysteine. Lancet 1977, 2(8035):432-434.
118. Peterson R G, Rumack B H: N-acetylcysteine for acetaminophen overdosage (cont.). N Engl J Med 1977, 296(9):515.
119. Peterson R G, Rumack B H: Treating acute acetaminophen poisoning with acetylcysteine. Jama 1977, 237(22):2406-2407.
120. Marquardt E D: Treatment of acetaminophen toxicity. Am J Hosp Pharm 1977, 34(8):805-806.
121. Maurer W G, Zeisler J: Intravenous acetylcysteine as treatment for acetaminophen overdose. Am J Hosp Pharm 1978, 35(9):1025, 1030.
122. Macy A M: Preventing hepatotoxicity in acetaminophen overdose. Am J Nurs 1979, 79(2):301-303.
123. Stewart D M, Dillman R O, Kim H S, Stewart K: Acetaminophen overdose: a growing health care hazard. Clin Toxicol 1979, 14(5):507-513.
124. Bailey B O: Acetaminophen hepatotoxicity and overdose. Am Fam Physician 1980, 22(1):83-87.
125. Black M: Acetaminophen hepatotoxicity. Gastroenterology 1980, 78(2):382-392.
126. Sellers E M, Freedman F: Treatment of acetaminophen poisoning. Can Med Assoc J 1981, 125(8):827-829.
127. Rumack B H, Peterson R C, Koch G G, Amara I A: Acetaminophen overdose. 662 cases with evaluation of oral acetylcysteine treatment. Arch Intern Med 1981, 141(3 Spec No):380-385.
128. Prescott L F, Critchley J A: The treatment of acetaminophen poisoning. Annu Rev Pharmacol Toxicol 1983, 23:87-101.
129. Miller L F, Rumack B H: Clinical safety of high oral doses of acetylcysteine. Semin Oncol 1983, 10(1 Suppl 1):76-85.
130. Rumack B H: Acetaminophen overdose in young children. Treatment and effects of alcohol and other additional ingestants in 417 cases. Am J Dis Child 1984, 138(5):428-433.
131. Rumack B H: Acetaminophen overdose in children and adolescents. Pediatr Clin North Am 1986, 33(3):691-701.
132. Davis M: Protective agents for acetaminophen overdose. Semin Liver Dis 1986, 6(2):138-147.
133. Larrauri A, Fabra R, Gomez-Lechon M J, Trullenque R, Castell J V: Toxicity of paracetamol in human hepatocytes. Comparison of the protective effects of sulfhydryl compounds acting as glutathione precursors. Mol Toxicol 1987, 1(4):301-311.
134. Slattery J T, Wilson J M, Kalhom T F, Nelson S D: Dose-dependent pharmacokinetics of acetaminophen: evidence of glutathione depletion in humans. Clin Pharmacol Ther 1987, 41:413-418.
135. Slattery J T, McRorie T I, Reynolds R, Kalhorn T F, Kharasch E D, Eddy A C: Lack of effect of cimetidine on acetaminophen disposition in humans. Clin Pharmacol Ther 1989, 46(5):591-597.
136. Burgunder J M, Varriale A, Lauterburg B H: Effect of N-acetylcysteine on plasma cysteine and glutathione following paracetamol administration. Eur J Clin Pharmacol 1989, 36(2):127-131.
137. Beckett G J, Donovan J W, Hussey A J, Proudfoot A T, Prescott L F: Intravenous N-acetylcysteine, hepatotoxicity and plasma glutathione S-transferase in patients with paracetamol overdosage. Hum Exp Toxicol 1990, 9(3):183-186.
138. Harrison P M, Keays R, Bray G P, Alexander G J, Williams R: Improved outcome of paracetamol-induced fulminant hepatic failure by late administration of acetylcysteine. Lancet 1990, 335(8705):1572-1573.
139. Bray G P, Mowat C, Muir D F, Tredger J M, Williams R: The effect of chronic alcohol intake on prognosis and outcome in paracetamol overdose. Hum Exp Toxicol 1991, 10(6):435-438.
140. Smilkstein M J, Bronstein A C, Linden C, Augenstein W L, Kulig K W, Rumack B H: Acetaminophen overdose: a 48-hour intravenous N-acetylcysteine treatment protocol. Ann Emerg Med 1991, 20(10):1058-1063.
141. Winkler E, Halkin H: Paracetamol overdose in Israel—1992. Isr J Med Sci 1992, 28(11):811-812.
142. Lee W M: Drug-induced hepatotoxicity. Ailment Pharmacology Theraphy 1993, 7:477-485.
143. Lee W M: Drug-induced hepatotoxicity. N Engl J Med 1995, 333(17): 1118-1127.
144. Lee W M: Management of acute liver failure. Semin Liver Dis 1996, 16(4):369-378.
145. De Roos F J, Hoffman R S: Drug-induced hepatotoxicity. N Engl J Med 1996, 334(13):863; discussion 864.
146. Perry H E, Shannon M W: Efficacy of oral versus intravenous N-acetylcysteine in acetaminophen overdose: results of an open-label, clinical trial [see comments]. J Pediatr 1998, 132(1):149-152.
147. Salgia A D, Kosnik S D: When acetaminophen use becomes toxic. Treating acute accidental and intentional overdose. Postgrad Med 1999, 105(4):81-84, 87, 90.
148. Ammenti A, Ferrante R, Spagna A: Renal impairment without hepatic damage after acetaminophen overdose. Pediatr Nephrol 1999, 13(3):271-272.
149. Buckley N A, Whyte I M, O'Connell D L, Dawson A H: Oral or intravenous N-acetylcysteine: which is the treatment of choice for acetaminophen (paracetamol) poisoning? J Toxicol Clin Toxicol 1999, 37(6):759-767.
150. Broughan T A, Soloway R D: Acetaminophen hepatoxicity. Dig Dis Sci 2000, 45(8):1553-1558.
151. Kearns G L, Leeder J S, Wasserman G S: Acetaminophen intoxication during treatment: what you don't know can hurt you. Clin Pediatr (Phila) 2000, 39(3):133-144.
152. Woo O F, Mueller P D, Olson K R, Anderson I B, Kim S Y: Shorter duration of oral N-acetylcysteine therapy for acute acetaminophen overdose. Ann Emerg Med 2000, 35(4):363-368.
153. Amirzadeh A, McCotter C: The intravenous use of oral acetylcysteine (mucomyst) for the treatment of acetaminophen overdose. Arch Intern Med 2002, 162(1):96-97.
154. Rumack B H: Acetaminophen hepatotoxicity: the first 35 years. J Toxicol Clin Toxicol 2002, 40(1):3-20.
155. Schmidt L E, Dalhoff K, Poulsen H E: Acute versus chronic alcohol consumption in acetaminophen-induced hepatotoxicity. Hepatology 2002, 35(4):876-882.
156. Jones A: Over-the-counter analgesics: a toxicology perspective. Am J Ther 2002, 9(3):245-257.
157. Kearns G L: Acetaminophen poisoning in children: treat early and long enough. J Pediatr 2002, 140(5):495-498.
158. Peterson J D, Herzenberg L A, Vasquez K, Waltenbaugh C: Glutathione levels in antigen-presenting cells modulate Th1 versus Th2 response patterns. Proc Natl Acad Sci USA 1998, 95(6):3071-3076.
159. Beckett G J, Chapman B J, Dyson E H, Hayes J D: Plasma glutathione S-transferase measurements after paracetamol overdose: evidence for early hepatocellular damage. Gut 1985, 26(1):26-31.
160. Montanini S, Sinardi D, Pratico C, Sinardi A U, Trimarchi G: Use of acetylcysteine as the life-saving antidote in Amanita phalloides (death cap) poisoning. Case report on 11 patients. Arzneimittelforschung 1999, 49(12): 1044-1047.
161. Hansen R M, Varma R R, Hanson G A: Gold induced hepatitis and pure red cell aplasia. Complete recovery after corticosteroid and N-acetylcysteine therapy. J Rheumatol 1991, 18(8):1251-1253.
162. Shartava A, Shah A K, Goodman S R: N-acetylcysteine and clotrimazole inhibit sickle erythrocyte dehydration induced by 1-chloro-2,4-dinitrobenzene. Am J Hematol 1999, 62(1):19-24.
163. Flanagan R J, Meredith T J: Use of N-acetylcysteine in clinical toxicology. Am J Med 1991, 91(3C):131s⁻¹³⁹S.
164. Harrison P M, Wendon J A, Gimson A E, Alexander G J, Williams R: Improvement by acetylcysteine of hemodynamics and oxygen transport in fulminant hepatic failure. N Engl J Med 1991, 324(26):1852-1857.
165. Lauterburg B H, Velez M E: Glutathione deficiency in alcoholics: risk factor for paracetamol hepatotoxicity. Gut 1988, 29:1153-1157.
166. Johnston S C, Pelletier L L, Jr.: Enhanced hepatotoxicity of acetaminophen in the alcoholic patient. Two case reports and a review of the literature. Medicine (Baltimore) 1997, 76(3):185-191.
167. Ozaras R, Tahan V, Aydin S, Uzun H, Kaya S, Senturk H: N-acetylcysteine attenuates alcohol-induced oxidative stress in the rat. World J Gastroenterol 2003, 9(1): 125-128.
168. Moss M, Guidot D M, Wong-Lambertina M, Ten Hoor T, Perez R L, Brown L A S: The effects of chronic alcohol abuse on pulmonary glutathione homeostasis. American Journal of Respiratory and Critical Care Medicine 2000, 161(2):414-419.
169. Golden M H, Ramdath D: Free radicals in the pathogenesis of kwashiorkor. Proc Nutr Soc 1987, 46(1):53-68.
170. Jackson A A: Blood glutathione in severe malnutrition in childhood. Trans R Soc Trop Med Hyg 1986, 80(6):911-913.
171. Reid M, Badalo A, Forrester T, Morlese J F, Frazer M, Heird W C, Jahoor F: In vivo rates of erythrocyte glutathione synthesis in children with severe protein-energy malnutrition. American Journal of Physiology—Endocrinology and Metabolism 2000, 278(3):E405-E412.
172. Lenhartz H, Ndasi R, Anninos A, Botticher D, Mayatepek E, Tetanye E, Leichsenring M: The clinical manifestation of the kwashiorkor syndrome is related to increased lipid peroxidation. J Pediatr 1998, 132(5):879-881.
173. Fechner A, Bohme C, Gromer S, Funk M, Schirmer R, Becker K: Antioxidant status and nitric oxide in the malnutrition syndrome kwashiorkor. Pediatr Res 2001, 49(2):237-243.
174. Anderson M T, Trudell J R, Voehringer D W, Tjioe I M, Herzenberg L A: An improved monobromobimane assay for glutathione utilizing tris-(2-carboxyethyl)phosphine as the reductant. Anal Biochem 1999, 272(1):107-109.
175. Horowitz J D, Henry C A, Syrjanen M L, Louis W J, Fish R D, Antman E M, Smith T W: Nitroglycerine/N-acetylcysteine in the management of unstable angina pectoris. Eur Heart J 1988, 9 Suppl A:95-100.
176. Horowitz J D, Henry C A, Syrjanen M L, Louis W J, Fish R D, Smith T W, Antman E M: Combined use of nitroglycerin and N-acetylcysteine in the management of unstable angina pectoris. Circulation 1988, 77(4):787-794.
177. Ardissino D, Merlini P A, Savonitto S, Demicheli G, Zanini P, Bertocchi F, Falcone C, Ghio S, Marinoni G, Montemartini C et al: Effect of transdermal nitroglycerin or N-acetylcysteine, or both, in the long-term treatment of unstable angina pectoris. J Am Coll Cardiol 1997, 29(5):941-947.
178. Ward K P, Arthur J R, Russell G, Aggett P J: Blood selenium content and glutathione peroxidase activity in children with cystic fibrosis, coeliac disease, asthma, and epilepsy. Eur JPediatr 1984, 142(1):21-24.
179. Altomare E, Colonna P, Dagostino C, Castellaneta G, Vendemiale G, Grattagliano 1, Cirelli F, Bovenzi F, Colonna L: High-dose antioxidant therapy during thrombolysis in patients with acute myocardial infarction. Current Therapeutic Research 1996, 57(2):131-141.
180. Boutis K, Shannon M: Nephrotoxicity after acute severe acetaminophen poisoning in adolescents. J Toxicol Clin Toxicol 2001, 39(5):441-445.
181. Klag M J, Whelton P K, Perneger T V: Analgesics and chronic renal disease. Curr Opin Nephrol Hypertens 1996, 5(3):236-241.
182. Duggin G G: Combination analgesic-induced kidney disease: the Australian experience. Am J Kidney Dis 1996, 28(1 Suppl l):S39-47.
183. Pemeger T V, Whelton P K, Klag M J: Risk of kidney failure associated with the use of acetaminophen, aspirin, and nonsteroidal antiinflammatory drugs. N Engl J Med 1994, 331(25):1675-1679.
184. Fored C M, Ejerblad E, Lindblad P, Fryzek J P, Dickman P W, Signorello L B, Lipworth L, Elinder C G, Blot W J, McLaughlin J K et al: Acetaminophen, aspirin, and chronic renal failure. NEngl J Med 2001, 345(25):1801-1808.
185. Durham J D, Caputo C, Dokko J, Zaharakis T, Pahlavan M, Keltz J, Dutka P, Marzo K, Maesaka J K, Fishbane S: A randomized controlled trial of N-acetylcysteine to prevent contrast nephropathy in cardiac angiography. Kidney Int 2002, 62(6):2202-2207.
186. Allaqaband S, Tumuluri R, Malik A M, Gupta A, Volkert P, Shalev Y, Bajwa T K: Prospective randomized study of N-acetylcysteine, fenoldopam, and saline for prevention of radiocontrast-induced nephropathy. Catheter Cardiovasc Interv 2002, 57(3):279-283.
187. Boccalandro F, Amhad M, Smalling R W, Sdringola S: Oral acetylcysteine does not protect renal function from moderate to high doses of intravenous radiographic contrast. Catheter Cardiovasc Interv 2003, 58(3):336-341.
188. Oldemeyer J B, Biddle W P, Wurdeman R L, Mooss A N, Cichowski E, Hilleman D E: Acetylcysteine in the prevention of contrast-induced nephropathy after coronary angiography. Am Heart J 2003, 146(6):E23.
189. Kefer J M, Hanet C E, Boitte S, Wilmotte L, De Kock M: Acetylcysteine, coronary procedure and prevention of contrast-induced worsening of renal function: which benefit for which patient? Acta Cardiol 2003, 58(6):555-560.
190. Briguori C, Manganelli F, Scarpato P, Elia P P, Golia B, Riviezzo G, Lepore S, Librera M, Villari B, Colombo A et al: Acetylcysteine and contrast agent-associated nephrotoxicity. J Am Coll Cardiol 2002, 40(2):298-303.
191. Baker C S, Wragg A, Kumar S, De Palma R, Baker L R, Knight C J: A rapid protocol for the prevention of contrast-induced renal dysfunction: the RAPPID study. J Am Coll Cardiol 2003, 41(12):2114-2118.
192. Birck R, Krzossok S, Markowetz F, Schnulle P, van der Woude F J, Braun C: Acetylcysteine for prevention of contrast nephropathy: meta-analysis. Lancet 2003, 362(9384):598-603.
193. Pannu N, Manns B, Lee H, Tonelli M: Systematic review of the impact of N-acetylcysteine on contrast nephropathy. Kidney Int 2004, 65(4):1366-1374.
194. Kshirsagar A V, Poole C, Motti A, Shoham D, Franceschini N, Tudor G, Agrawal M, Denu-Ciocca C, Magnus Ohman E, Finn W F: N-acetylcysteine for the prevention of radiocontrast induced nephropathy: a meta-analysis of prospective controlled trials. J Am Soc Nephrol 2004, 15(3):761-769.
195. Davies S J, D'Sousa R, Philips H, Mattey D, Hiley C, Hayes J D, Aber G M, Strange R C: Localisation of alpha, mu and pi class glutathione S-transferases in kidney: comparison with CuZn superoxide dismutase. Biochim Biophys Acta 1993, 1157(2):204-208.
196. St Peter S D, Imber C J, Jones D C, Fuggle S V, Watson C J, Friend P J, Marshall S E:

Genetic determinants of delayed graft function after kidney transplantation. Transplantation 2002, 74(6):809-813.
197. McKay S, Morlese J, Balasubramanyam A, Reeds P, Jahoor F: Impact of glycine and cysteine supplementation on oxidative stress and glutathione synthesis in type 2 diabetes. A stable isotope approach. In: 5th Inter Synp on Amino Acid/Protein Metabolism in Health and Disease. 1999; Perugia, Italy: Diabetes, Nutrition & Metabolism; 1999: 174.
198. Ristoff E, Larsson A: Oxidative stress in inborn errors of metabolism: lessons from glutathione deficiency. J Inherit Metab Dis 2002, 25(3):223-226.
199. RistoffE, Mayatepek E, Larsson A: Long-term clinical outcome in patients with glutathione synthetase deficiency. J Pediatr 2001, 139(1):79-84.
200. Heinig J H, Pedersen B, Andersen I, Dalgaard C E, Rasmussen O, Weeke E R, Enk B: [The mucolytic effects of acetylcysteine compared with bromhexine and a placebo in patients with chronic bronchitis]. Ugeskr Laeger 1985, 147(46):3694-3697.
201. Millar A B, Pavia D, Agnew J E, Lopez-Vidriero M T, Lauque D, Clarke S W: Effect of oral N-acetylcysteine on mucus clearance. Br J Dis Chest 1985, 79(3):262-266.
202. McGavin C: Oral N-acetylcysteine and exacerbation rates in patients with chronic bronchitis and severe airways obstruction. British Thoracic Society Research Committee. Thorax 1985, 40(11):832-835.
203. Bibi H, Seifert B, Oullette M, Belik J: Intratracheal N-acetylcysteine use in infants with chronic lung disease. Acta Paediatr 1992, 81(4):335-339.
204. Dueholm M, Nielsen C, Thorshauge H, Evald T, Hansen N C, Madsen H D, Maltbaek N: N-acetylcysteine by metered dose inhaler in the treatment of chronic bronchitis: a multi-centre study. Respir Med 1992, 86(2):89-92.
205. Poole P J, Black P N: Oral mucolytic drugs for exacerbations of chronic obstructive pulmonary disease: systematic review. Bmj 2001, 322(7297):1271-1274.
206. Stey C, Steurer J, Bachmann S, Medici T C, Tramer M R: The effect of oral N-acetylcysteine in chronic bronchitis: a quantitative systematic review. Eur Respir J 2000, 16(2):253-262.
207. Grandjean E M, Berthet P, Ruffiann R, Leuenberger P: Cost-effectiveness analysis of oral N-acetylcysteine as a preventive treatment in chronic bronchitis. Pharmacol Res 2000, 42(1):39-50.
208. Grandjean E M, Berthet P, Ruffinann R, Leuenberger P: Efficacy of oral long-term N-acetylcysteine in chronic bronchopulmonary disease: a meta-analysis of published double-blind, placebo-controlled clinical trials. Clin Ther 2000, 22(2):209-221.
209. Ekberg-Jansson A, Larson M, MacNee W, Tunek A, Wahlgren L, Wouters E F, Larsson S: N-isobutyrylcysteine, a donor of systemic thiols, does not reduce the exacerbation rate in chronic bronchitis. Eur Respir J 1999, 13(4):829-834.
210. Kasielski M, Nowak D: Long-term administration of N-acetylcysteine decreases hydrogen peroxide exhalation in subjects with chronic obstructive pulmonary disease. Respir Med 2001, 95(6):448-456.
211. Suter P M, Domenighetti G, Schaller M-D, Layerriere M-C, Ritz R, Perret C: N-acetylcysteine enhances recovery from acute lung injury in man. A randomized, double-blind, placebo-controlled clinical study. Chest 1994, 105:190-194.
212. Zimmerman R J, Marafino Jr. B J, Chan A, Landre P, Winkelhake J L: The role of oxidant injury in tumor cell sensitivity to recombinant human tumor necrosis factor in vivo. Implications for mechanism of action. Journal of Immunology 1989, 142:1405-1409.
213. Borregaard N, Jensen H S, Bjerrum O W: Prevention of tissue damage: inhibition of myeloperoxidase mediated inactivation of alpha 1-proteinase inhibitor by N-acetyl cysteine, glutathione, and methionine. Agents Actions 1987, 22(3-4):255-260.
214. De Backer W A, Amsel B, Jorens P G, Bossaert L, Hiemstra P S, van Noort P, van Overveld F J: N-acetylcysteine pretreatment of cardiac surgery patients influences plasma neutrophil elastase and neutrophil influx in bronchoalveolar lavage fluid. Intensive Care Med 1996, 22(9):900-908.
215. Laurent T, Markert M, Feihi F, Schaller M D, Perret C: Oxidant-antioxidant balance in granulocytes during ARDS. Effect of N-acetylcysteine. Chest 1996, 109(1): 163-166.
216. Eklund A, Eriksson O, Hakansson L, Larsson K, Ohlsson K, Venge P, Bergstrand H, Bjornson A, Brattsand R, Glennow C et al: Oral N-acetylcysteine reduces selected humoral markers of inflammatory cell activity in BAL fluid from healthy smokers: correlation to effects on cellular variables. Eur Respir J 1988, 1(9):832-838.
217. Moriuchi H, Zaha M, Fukumoto T, Yuizono T: Activation of polymorphonuclear leukocytes in oleic acid-induced lung injury. Intensive Care Med 1998, 24(7):709-715.
218. Villa P, Saccani A, Sica A, Ghezzi P: Glutathione protects mice from lethal sepsis by limiting inflammation and potentiating host defense. J Infect Dis 2002, 185(8):1115-1120.
219. Domenighetti G, Suter P M, Schaller M D, Ritz R, Perret C: Treatment with N-acetylcysteine during acute respiratory distress syndrome: a randomized, double-blind, placebo-controlled clinical study. J Crit Care 1997, 12(4):177-182.
220. Heller A R, Groth G, Heller S C, Breitkreutz R, Nebe T, Quintel M, Koch T: N-acetylcysteine reduces respiratory burst but augments neutrophil phagocytosis in intensive care unit patients. Crit Care Med 2001, 29(2):272-276.
221. Bernard G R: N-acetylcysteine in experimental and clinical acute lung injury. Am J Med 1991, 91(3C):54S-59S.
222. Bernard G R, Wheeler A P, Arons M M, Morris P E, Paz H L, Russell J A, Wright P E, Bernard G R, Arons M M, Wheeler A P et al: A trial of antioxidants N-acetylcysteine and procysteine in ARDS. Chest 1997, 112(1):164-172.
223. Jepsen S, Herlevsen P, Knudsen P, Bud M I, Klausen N O: Antioxidant treatment with N-acetylcysteine during adult respiratory distress syndrome: a prospective, randomized, placebo-controlled study. Crit Care Med 1992, 20(7):918-923.
224. Konrad F, Schoenberg M H, Wiedmann H, Kilian J, Georgieff M: [The application of n-acetylcysteine as an antioxidant and mucolytic in mechanical ventilation in intensive care patients. A prospective, randomized, placebo-controlled, double-blind study]. Anaesthesist 1995, 44(9):651-658.
225. Droge W, Breitkreutz R: N-acetyl-cysteine in the therapy of HIV-positive patients. Curr Opin Clin Nutr Metab Care 1999, 2(6):493-498.
226. Clotet B, Gomez M, Ruiz L, Sirera G, Romeu J: Lack of short-term efficacy of N-acetyl-L-cysteine in human immunodeficiency virus-positive patients with CD4 cell counts <250/mm(3) {Letter}. Journal of Acquired Immune Deficiency Syndromes and Human Retrovirology 1995, 9(1):98-99.
227. Spada C, Treitinger A, Reis M, Masokawa I Y, Verdi J C, Luiz M C, Silveira M V, Michelon C M, Avila-Junior S, Gil D O et al: The effect of N-acetylcysteine supplementation upon viral load, CD4, CD8, total lymphocyte count and hematocrit in individuals undergoing antiretroviral treatment. Clin Chem Lab Med 2002, 40(5):452-455.
228. Verhagen H, Hageman G J, Rauma A L, Versluis-de Haan G, van Herwijnen M H, de Groot J, Torronen R, Mykkanen H: Biomonitoring the intake of garlic via urinary excretion of allyl mercapturic acid. Br JNutr 2001, 86 Suppl I:S111-114.
229. Droge W, Eck H-P, Gmunder H, Mihm S: Modulation of lymphocyte functions and immune responses by cysteine and cysteine derivatives. Am J Med 1991, 91 (Suppl. 3C): 140S-144S.
230. Roederer M, Staal F J, Raju P A, Ela S W, Herzenberg L A: Cytokine-stimulated human immunodeficiency virus replication is inhibited by N-acetyl-L-cysteine. Proc Natl Acad Sci USA 1990, 87(12):4884-4888.
231. Bertini R, Howard O M, Dong H F, Oppenheim J J, Bizzarri C, Sergi R, Caselli G, Pagliei S, Romines B, Wilshire J A et al: Thioredoxin, a redox enzyme released in infection and inflammation, is a unique chemoattractant for neutrophils, monocytes, and T cells. J Exp Med 1999, 189(11):1783-1789.
232. Nakamura H, Masutani H, Yodoi J: Redox Imbalance and Its Control in HIV Infection. Antioxid Redox Signal 2002, 4(3):455-464.
233. Roederer M, Ela S W, Staal FJ T, Herzenberg L A, Herzenberg L A: N-Acetylcysteine: a new approach to anti-HIV therapy. AIDS Res Human Retrov 1992, 8(2):209-217.
234. Herzenberg L A, De Rosa S C, Herzenberg L A: Low Glutathione Levels in CD4 T Cells Predict Poor Survival in AIDS; N-Acetyclsteine May Improve Survival. In: Oxidative Stress in Cancer, AIDS and Neuodegerative Diseases. Edited by Montagnier L, Pasquier C, vol. 1: Marcel Dekker, Inc.; 1998: 379-387.
235. English M, Sauerwein R, Waruiru C, Mosobo M, Obiero J, Lowe B, Marsh K: Acidosis in severe childhood malaria. Qim 1997, 90(4):263-270.
236. Marsh K, Forster D, Waruiru C, Mwangi I, Winstanley M, Marsh V, Newton C, Winstanley P, Warn P, Peshu N et al: Indicators of life-threatening malaria in African children. N Engl J Med 1995, 332(21):1399-1404.
237. Day N P, Phu N H, Mai N T, Chau T T, Loc P P, Chuong L V, Sinh D X, Holloway P, Hien T T, White N J: The pathophysiologic and prognostic significance of acidosis in severe adult malaria. Crit Care Med2000, 28(6):1833-1840.
238. Grau G E, Fajardo L F, Piguet P F, Allet B, Lambert P H, Vassalli P: Tumor necrosis factor (cachectin) as an essential mediator in murine cerebral malaria. Science 1987, 237(4819):1210-1212.
239. Day N P, Hien T T, Schollaardt T, Loc P P, Chuong L V, Chau T T, Mai N T, Phu N H, Sinh D X, White N J et al: The prognostic and pathophysiologic role of pro- and antiinflammatory cytokines in severe malaria. JInfect Dis 1999, 180(4): 1288-1297.
240. Gruarin P, Primo L, Ferrandi C, Bussolino F, Tandon N N, Arese P, Ulliers D, Alessio M: Cytoadherence of Plasmodium falciparum-infected erythrocytes is mediated by a redox-dependent conformational fraction of CD36. J Immunol 2001, 167(11):6510-6517.
241. Schulz J B, Lindenau J, Seyfried J, Dichgans J: Glutathione, oxidative stress and neurodegeneration. Eur J Biochem 2000, 267(16):4904-4911.
242. Louwerse E S, Weverling G J, Bossuyt P M, Meyjes F E, de Jong J M: Randomized, double-blind, controlled trial of acetylcysteine in amyotrophic lateral sclerosis. Arch Neurol 1995, 52(6):559-564.
243. Kopke R D, Coleman J K M, Huang X, Weisskopf P A, Jackson R L, Liu J J, Hoffer M E, Wood K A, Kil J, Van De Water T R: Novel Strategies to Prevent and Reverse Noise-induced Hearing Loss—Chapter 4. In: Treatments and Protection In: Noise Induced Hearing Loss Basic Mechanisms, Prevention and Control. Edited by Don Henderson D P, Richard Kopke, Richard Salvi and Roger Hamernik. London, England: Noise Research Network Publications; 2001: 231-253.
244. Kopke R D, Allen K A, Henderson D: Toxins and trauma share common pathways in hair cell death. A Radical Demise. In: Ototoxicity: Basic Sciences and Clinical Applications. Edited by D. Henderson R, A Quaranta, S. McFadden, and R. Buckard, vol. 884. New York: Annals of the New York Academy of Sciences.; 1999.
245. Kopke R D, Liu W, Gabaizadeh R, Jacono A, Feghali J, Spray D, Garcia P, Steinman H, Malgrange B, Ruben R J et al: Use of organotypic cultures of Corti's organ to study the protective effects of antioxidant molecules on cisplatin-induced damage of auditory hair cells. Am J Otol 1997, 18(5):559-571.
246. Kopke R D, Weisskopf P A, Boone J L, Jackson R L, Wester D C, Hoffer M E, Lambert D C, Charon C C, Ding D L, McBride D: Reduction of noise-induced hearing loss using L-NAC and salicylate in the chinchilla. Hear Res 2000, 149(1-2):138-146.
247. Sha S H, Schacht J: Antioxidants attenuate gentamicin-induced free radical formation in vitro and ototoxicity in vivo: D-methionine is a potential protectant. Hear Res 2000, 142(1-2):34-40.
248. Kopke R D, Coleman J K, Liu J, Campbell K C, Riffenburgh R H: Candidate's Thesis: Enhancing Intrinsic Cochlear Stress Defenses to Reduce Noise-Induced Hearing Loss. Laryngoscope 2002, 112(9):1515-1532.
249. Schmidt L E, Dalhoff K P: [Side-effects of N-acetylcysteine treatment in patients with paracetamol poisoning]. Ugeskr Laeger 1999, 161(18):2669-2672.
250. Walton N G, Mann T A, Shaw K M: Anaphylactoid reaction to N-acetylcysteine. Lancet 1979, 2(8155):1298.
251. Vale J A, Wheeler D C: Anaphylactoid reaction to acetylcysteine. Lancet 1982, 2(8305):988.
252. Bonfiglio M F, Traeger S M, Hulisz D T, Martin B R: Anaphylactoid reaction to intravenous acetylcysteine associated with electrocardiographic abnormalities. Ann Pharmacother 1992, 26(1):22-25.
253. Stavem K: [Anaphylactic reaction to N-acetylcysteine after poisoning with paracetamol]. Tidsskr Nor Laegeforen 1997, 117(14):2038-2039.
254. Bailey B, McGuigan M A: Management of anaphylactoid reactions to intravenous N-acetylcysteine. Ann Emerg Med 1998, 31(6):710-715.
255. Huitema A D, Soesan M, Meenhorst P L, Koks C H, Beijnen J H: A dose-dependent delayed hypersensitivity reaction to acetaminophen after repeated acetaminophen intoxications. Hum Exp Toxicol 1998, 17(7):406-408.
256. Bateman D N, Woodhouse K W, Rawlins M D: Adverse reactions to N-acetylcysteine. Hum Toxicol 1984, 3(5):393-398.
257. Sandstrom P A, Murray J, Folks T M, Diamond A M: Antioxidant defenses influence HIV-1 replication and associated cytopathic effects. Free Radical Biology and Medicine 1998, 24(9):1485-1491.
258. Magee E A, Richardson C J, Hughes R, Cummings J H: Contribution of dietary protein to sulfide production in the large intestine: an in vitro and a controlled feeding study in humans. Am J Clin Nutr 2000, 72(6):1488-1494.
259. Andus T, Gross V: Etiology and pathophysiology of inflammatory bowel disease—environmental factors. Hepatogastroenterology 2000, 47(31):29-43.
260. Babidge W, Millard S, Roediger W: Sulfides impair short chain fatty acid beta-oxidation at acyl-CoA dehydrogenase level in colonocytes: implications for ulcerative colitis. Mol Cell Biochem 1998, 181(1-2):117-124.
261. Pitcher M C, Beatty E R, Cummings J H: The contribution of sulphate reducing bacteria and 5-aminosalicylic acid to faecal sulphide in patients with ulcerative colitis. Gut 2000, 46(1):64-72.
262. Andrus J P, Herzenberg L A, DeRosa S C: Effects of legislation restricting pack sizes of paracetamol on self poisoning. Paracetamol should be packaged with its antidote. Bmj 2001, 323(7313):634.
263. Andrus J P, De Rosa S C, Herzenberg L A, Herzenberg L A: Fomulation for the prevention of Acetaminophen Toxicity. In USA: BioAdvantex Pharma.; 2003.
264. Hazelton G A, Hjelle J J, Klaassen C D: Effects of cysteine pro-drugs on acetaminophen-induced hepatotoxicity. J Pharmacol Exp Ther 1986, 237(1):341-349.
265. Grant P R, Black A, Garcia N, Prieto J, Garson J A: Combination therapy with interferon-alpha plus N-acetyl cysteine for chronic hepatitis C: a placebo controlled double-blind multicentre study. J Med Virol 2000, 61(4):439-442.
266. Reinhart K, Spies C D, Meier-Hellmann A, Bredle D L, Hannemann L, Specht M, Schaffartzik W: N-acetylcysteine preserves oxygen consumption and gastric mucosal pH during hyperoxic ventilation. Am JRespir Crit Care Med 1995, 151(3 Pt 1):773-779.
267. Kinnunen J, Pietila J, Ahovuo J, Mankinen P, Tervahartiala P: Double contrast barium meal and acetylcysteine. Eur J Radiol 1989, 9(4):258-259.
268. Demirturk L, Yazgan Y, Tarcin O, Ozel M, Diler M, Oncul O, Yildirim S: Does N-acetyl cystein affect the sensitivity and specificity of Helicobacter pylori stool antigen test? Helicobacter 2003, 8(2):120-123.
269. Weber S, Auclert L, Touiza K, Pellois A, Guerin C, Blin P, Guerin F: [Evaluation of tolerance during intravenous administration of low dose of isosorbide dinitrate in the treatment of unstable angina]. Arch Mal Coeur Vaiss 1992, 85(1):59-65.
270. Fischer U M, Tossios P, Huebner A, Geissler H J, Bloch W, Mehlhom U: Myocardial apoptosis prevention by radical scavenging in patients undergoing cardiac surgery. J Thorac Cardiovasc Surg 2004, 128(1): 103-108.
271. Drager L F, Andrade L, Barros de Toledo J F, Laurindo F R, Machado Cesar L A, Seguro A C: Renal effects of N-acetylcysteine in patients at risk for contrast nephropathy: decrease in oxidant stress-mediated renal tubular injury. Nephrol Dial Transplant 2004, 19(7):1803-1807.
272. Goldenberg I, Shechter M, Matetzky S, Jonas M, Adam M, Pres H, Elian D, Agranat O, Schwammenthal E, Guetta V: Oral acetylcysteine as an adjunct to saline hydration for the prevention of contrast-induced nephropathy following coronary angiography. A randomized controlled trial and review of the current literature. Eur Heart J 2004, 25(3):212-218.
273. Scholze A, Rinder C, Beige J, Riezler R, Zidek W, Tepel M: Acetylcysteine reduces plasma homocysteine concentration and improves pulse pressure and endothelial function in patients with end-stage renal failure. Circulation 2004, 109(3):369-374.
274. Ratjen F, Wonne R, Posselt H G, Stover B, Hofmann D, Bender S W: A double-blind placebo controlled trial with oral ambroxol and N-acetylcysteine for mucolytic treatment in cystic fibrosis. Eur J Pediatr 1985, 144(4):374-378.
275. Gotz M, Kraemer R, Kerrebijn K F, Popow C: Oral acetylcysteine in cystic fibrosis. A co-operative study. Eur J Respir Dis Suppl 1980, 111:122-126.
276. Friedman A N, Bostom A G, Laliberty P, Selhub J, Shemin D: The effect of N-acetylcysteine on plasma total homocysteine levels in hemodialysis: A randomized, controlled study. Am J Kidney Dis 2003, 41(2):442-446.
277. Wiklund O, Fager G, Andersson A, Lundstam U, Masson P, Hultberg B: N-acetylcysteine treatment lowers plasma homocysteine but not serum lipoprotein(a) levels. Atherosclerosis 1996, 119(1):99-106.
278. Koechlin C, Couillard A, Simar D, Cristol J P, Bellet H, Hayot M, Prefaut C: Does oxidative stress alter quadriceps endurance in chronic obstructive pulmonary disease? Am J Respir Crit Care Med 2004, 169(9):1022-1027.
279. Ahola T, Lapatto R, Raivio K O, Selander B, Stigson L, Jonsson B, Jonsbo F, Esberg G, Stovring S, Kjartansson S et al: N-acetylcysteine does not prevent bronchopulmonary dysplasia in immature infants: a randomized controlled trial. J Pediatr 2003, 143(6):713-719.
280. Jepsen S, Nielsen P H, Klaerke A, Nielsen S T, Simonsen 0: Peroral N-acetylcysteine as prophylaxis against bronchopulmonary complications of pulmonary surgery. Scand J Thorac Cardiovasc Surg 1989, 23(2):185-188.
281. Peake S L, Moran J L, Leppard P I: N-acetyl-L-cysteine depresses cardiac performance in patients with septic shock. Crit Care Med 1996, 24(8): 1302-1310.
282. Droge W, Breitkreutz R: Glutathione and immune function. Proc Nutr Soc 2000, 59(4):595-600.
283. Barditch-Crovo P, Noe D, Skowron G, Lederman M, Kalayjian R C, Borum P, Buier R, Rowe W B, Goldberg D, Lietman P: A phase I/II evaluation of oral L-2-oxothiazolidine-4-carboxylic acid in asymptomatic patients infected with human immunodeficiency virus. J Clin Pharmacol 1998, 38(4):357-363.
284. Akerlund B, Tynell E, Bratt G, Bielenstein M, Lidman C: N-acetylcysteine treatment and the risk of toxic reactions to trimethoprim-sulphamethoxazole in primary Pneumocystis carinii prophylaxis in HIV-infected patients. J Infect 1997, 35(2):143-147.
285. Molnar Z, MacKinnon K L, Shearer E, Lowe D, Watson I D: The effect of N-acetylcysteine on total serum anti-oxidant potential and urinary albumin excretion in critically ill patients. Intensive Care Med 1998, 24(3):230-235.
286. Medved I, Brown M J, Bjorksten A R, Murphy K T, Petersen A C, Sostaric S, Gong X, McKenna M J: N-acetylcysteine enhances muscle cysteine and glutathione availability and attenuates fatigue during prolonged exercise in endurance-trained individuals. J Appl Physiol 2004.
287. Furst D E, Clements P J, Harris R, Ross M, Levy J, Paulus H E: Measurement of clinical change in progressive systemic sclerosis: a 1 year double-blind placebo-controlled trial of N-acetylcysteine. Ann Rheum Dis 1979, 38(4):356-361.
288. James L P, Wells E, Beard R H, Farrar H C: Predictors of outcome after acetaminophen poisoning in children and adolescents. J Pediatr 2002, 140(5):522-526.
289. Prescott L F: New approaches in managing drug overdosage and poisoning. Br Med J (Clin Res Ed) 1983, 287(6387):274-276.
290. Bondy S C: Ethanol toxicity and oxidative stress. (Comment). Toxicol Lett 1992, 63:231-241.
291. Loguercio C, Nardi G, Argenzio F, Aurilio C, Petrone E, Grella A, Del Vecchio Blanco C, Coltorti M: Effect of S-adenosyl-L-methionine administration on red blood cell cysteine and glutathione levels in alcoholic patients with and without liver disease. Alcohol Alcohol 1994, 29(5):597-604.
292. Sprietsma J E: Modern diets and diseases: NO-zinc balance. Under Thl, zinc and nitrogen monoxide (NO) collectively protect against viruses, AIDS, autoimmunity, diabetes, allergies, asthma, infectious diseases, atherosclerosis and cancer. Med Hypotheses 1999, 53(1):6-16.
293. Tripi S, Di Gaetano G, Soresi M, Carroccio A, Bonfissuto G, Savi A, Vuturo O, Montalto G: Acetylcysteine therapy for chronic hepatitis C: Are its effects synergistic with interferon alpha? A pilot study. Clinical Drug Investigation 1998, 16(4):297-302.
294. Di Bisceglie A M: Hepatitis C. Lancet 1998, 351(9099):351-355.
295. Suarez M, Beloqui O, Ferrer J V, Gil B, Qian C, Garcia N, Civeira P, Prieto J: Glutathione depletion in chronic hepatitis C. Internatl Hepatol Commun 1993, 1:215-221.
296. Grattagliano I, Vendemiale G, Sabba C, Buonamico P, Altomare E: Oxidation of circulating proteins in alcoholics: role of acetaldehyde and xanthine oxidase. Journal of Hepatology 1996, 25(1):28-36.
297. Beloqui O, Prieto J, Suarez M, Gil B, Qian C H, Garcia N, Civeira M P: N-acetyl cysteine enhances the response to interferon-alpha in chronic hepatitis C: a pilot study. J Interferon Res 1993, 13(4):279-282.
298. Barbaro G, Di Lorenzo G, Soldini M, Parrotto S, Bellomo G, Belloni G, Grisorio B, Barbarini G: Hepatic glutathione deficiency in chronic hepatitis C: quantitative evaluation in patients who are HIV positive and HIV negative and correlations with plasmatic and lymphocytic concentrations and with the activity of the liver disease. Am J Gastroenterol 1996, 91(12):2569-2573.
299. Barbaro G, Di Lorenzo G, Soldini M, Bellomo G, Belloni G, Grisorio B, Barbarini G: Vagal system impairment in human immunodeficiency virus-positive patients with chronic hepatitis C: Does hepatic glutathione deficiency have a pathogenetic role? Scandinavian Journal of Gastroenterology 1997, 32(12):1261-1266.
300. Kes P: Hyperhomocysteinemia in end-stage renal failure. Acta Med Croatica 2000, 54(4-5):175-181.
301. Moberly J B, Logan J, Borum P R, Story K O, Webb L E, Jassal S V, Mupas L, Rodela H, Alghamdi G A, Moran J E et al: Elevation of whole-blood glutathione in peritoneal dialysis patients by L-2-oxothiazolidine-4-carboxylate, a cysteine prodrug (Procysteine(R)). Journal of the American Society of Nephrology 1998, 9(6): 1093-1099.
302. Bostom A G, Shemin D, Yoburn D, Fisher D H, Nadeau M R, Selhub J: Lack of effect of oral N-acetylcysteine on the acute dialysis-related lowering of total plasma homocysteine in hemodialysis patients. Atherosclerosis 1996, 120(1-2):241-244.
303. Boushey C J, Beresford S A, Omenn G S, Motulsky A G: A quantitative assessment of plasma homocysteine as a risk factor for vascular disease. Probable benefits of increasing folic acid intakes. Jama 1995, 274(13):1049-1057.
304. Morrison J A, Jacobsen D W, Sprecher D L, Robinson K, Khoury P, Daniels S R: Serum glutathione in adolescent males predicts parental coronary heart disease. Circulation 1999, 100(22):2244-2247.
305. Sasazuki S, Kodama H, Yoshimasu K, Liu Y, Washio M, Tanaka K, Tokunaga S, Kono S, Arai H, Doi Y et al: Relation between green tea consumption and the severity of coronary atherosclerosis among Japanese men and women. Ann Epidemiol 2000, 10(6):401-408.
306. Tice J A, Ross E, Coxson P G, Rosenberg I, Weinstein M C, Hunink M G, Goldman P A, Williams L, Goldman L: Cost-effectiveness of vitamin therapy to lower plasma homocysteine levels for the prevention of coronary heart disease: effect of grain fortification and beyond. Jama 2001, 286(8):936-943.
307. Andrews N P, Prasad A, Quyyumi A A: N-acetylcysteine improves coronary and peripheral vascular function. J Am Coll Cardiol 2001, 37(1):117-123.
308. Prasad A, Andrews N P, Padder F A, Husain M, Quyyumi A A: Glutathione reverses endothelial dysfunction and improves nitric oxide bioavailability. Journal of the American College of Cardiology 1999, 34(2):507-514.
309. Dincer Y, Akcay T, Konukogku D, Hatemi H: Erythrocyte susceptibility to lipid peroxidation in patients with coronary atherosclerosis. Acta Medica Okayama 1999, 53(6):259-264.
310. Usal A, Acarturk E, Yuregir G T, Unlukurt I, Demirci C, Kurt H I, Birand A: Decreased glutathione levels in acute myocardial infarction. Jpn Heart J 1996, 37(2):177-182.
311. Yucel D, Aydogdu S, Cehreli S, Saydam G, Canatan H, Senes M, Topkaya B C, Nebioglu S: Increased oxidative stress in dilated cardiomyopathic heart failure. Clinical Chemistry 1998, 44(1):148-154.
312. Straface E, Rivabene R, Masella R, Santulli M, Paganelli R, Malorni W: Structural changes of the erythrocyte as a marker of non-insulin-dependent diabetes: protective effects of N-acetylcysteine. Biochem Biophys Res Commun 2002, 290(5):1393-1398.
313. Jain S K, McVie R, Jaramillo J J, Palmer M, Smith T: Effect of modest vitamin E supplementation on blood glycated hemoglobin and triglyceride levels and red cell indices in type I diabetic patients. J Am Coll Nutr 1996, 15(5):458-461.
314. Yoshida K, Hirokawa J, Tagami S, Kawakami Y, Urata Y, Kondo T: Weakened cellular scavenging activity against oxidative stress in diabetes mellitus: regulation of glutathione synthesis and efflux. Diabetologia 1995, 38(2):201-210.
315. Konukoglu D, Hatemi H, Ozer E M, Gonen S, Akcay T: The erythrocyte glutathione levels during oral glucose tolerance test. Journal of Endocrinological Investigation 1997, 20(8):471-475.
316. Jain S K, Krueger K S, McVie R, Jaramillo J J, Palmer M, Smith T: Relationship of blood thromboxane-B2 (TxB2) with lipid peroxides and effect of vitamin E and placebo supplementation on TxB2 and lipid peroxide levels in type 1 diabetic patients. Diabetes Care 1998, 21(9):1511-1516.
317. Graber R, Farine J C, Fumagalli I, Tatti V, Losa G A: Apoptosis and oxidative status in peripheral blood mononuclear cells of diabetic patients. Apoptosis 1999, 4(4):263-270.
318. Murakami K, Kondo T, Ohtsuka Y, Fujiwara Y, Shimada M, Kawakami Y: Impairment of glutathione metabolism in erthrocytes from patients with diabetes mellitus. Metabolism, Clinical & Experimental 1989, 38:753-758.
319. Konukoglu D, Akcay T, Dincer Y, Hatemi H: The susceptibility of red blood cells to autoxidation in type 2 diabetic patients with angiopathy. Metabolism 1999, 48(12):1481-1484.
320. Tessier D, Khalil A, Fulop T: Effects of an oral glucose challenge on free radicals/antioxidants balance in an older population with type II diabetes. Journals of Gerontology Series A—Biological Sciences and Medical Sciences 1999, 54(11):M541-M545.
321. Jain S K, McVie R: Hyperketonemia can increase lipid peroxidation and lower glutathione levels in human erythrocytes in vitro and in type 1 diabetic patients. Diabetes 1999, 48(9):1850-1855.
322. Jain S K, McVie R, Smith T: Vitamin E supplementation restores glutathione and malondialdehyde to normal concentrations in erythrocytes of type 1 diabetic children. Diabetes Care 2000, 23(9):1389-1394.
323. Rizvi S I, Zaid M A: Intracellular reduced glutathione content in normal and type 2 diabetic erythrocytes: effect of insulin and (−)epicatechin. J Physiol Pharmacol 2001, 52(3):483-488.
324. Miller M: [Clinical experimentation using a combination with antibiotic and mucolytic activity in the treatment of respiratory and oto-rhino-laryngologic infections with allergic components]. Brux Med 1975, 55(6):349-351.
325. Kupczyk M, Kuna P: [Mucolytics in acute and chronic respiratory tract disorders. II. Uses for treatment and antioxidant properties]. Pol Merkuriusz Lek 2002, 12(69):248-252.
326. Kupczyk M, Kuna P: [Mucolytics in acute and chronic respiratory tract disorders. I. Pathophysiology and mechanisms of action]. Pol Merkuriusz Lek 2002, 12(69):245-247.
327. Ruffmann R, Wendel A: GSH rescue by N-acetylcysteine. Klinische Wochenschrift 1991, 69(18):857-862.
328. Kelly F J: Gluthathione: in defence of the lung. Food Chem Toxicol 1999, 37(9-10):963-966.
329. White A C, Thannickal V J, Fanburg B L: Glutathione deficiency in human disease. J Nutr Biochem 1994, 5:218-226.
330. Melillo G, Chiummariello A, Scala G: [On the use of a new molecular combination of acetylcysteine with thiamphenicol glycinate in bronchopulmonary suppurations]. G Ital Chemioter 1966, 13(1):156-160.
331. Mayaud C, Lentschner C, Bouchoucha S, Marsac J: [Thiamphenicol glycinate acetylcysteinate in the treatment of acute respiratory infections with mucostasis]. Eur J Respir Dis Suppl 1980, 111:70-73.
332. Bernard G R: Potential of N-acetylcysteine as treatment for the adult respiratory distress syndrome. Eur Respir J Suppl 1990, 11:496s-498s.
333. Christman B W, Bernard G R: Antilipid mediator and antioxidant therapy in adult respiratory distress syndrome. New Horiz 1993, 1(4):623-630.
334. Gillissen A, Nowak D: Characterization of N-acetylcysteine and ambroxol in anti-oxidant therapy. Respiratory Medicine 1998, 92(4):609-623.
335. Behr J, Maier K, Degenkolb B, Krombach F, Vogelmeier C: Antioxidative and clinical effects of high-dose N-acetylcysteine in fibrosing alveolitis. Adjunctive therapy to maintenance immunosuppression. Am J Respir Crit Care Med 1997, 156(6):1897-1901.
336. Behr J, Degenkolb B, Krombach F, Vogelmeier C: Intracellular glutathione and bronchoalveolar cells in fibrosing alveolitis: effects of N-acetylcysteine. Eur Respir J 2002, 19(5):906-911.
337. Dick C A, Brown D M, Donaldson K, Stone V: The role of free radicals in the toxic and inflammatory effects of four different ultrafine particle types. Inhal Toxicol 2003, 15(1):39-52.
338. Riise G C, Larsson S, Larsson P, Jeansson S, Andersson B A: The intrabronchial microbial flora in chronic bronchitis patients: a target for N-acetylcysteine therapy? Eur Respir J 1994, 7(1):94-101.
339. Lamson D W, Brignall M S: The use of nebulized glutathione in the treatment of emphysema: a case report [In Process Citation]. Altern Med Rev 2000, 5(5):429-431.
340. Chikina S, lagmurov B, Kopylev I D, Soodaeva S K, Chuchalin A G: [N-Acetylcysteine: low and high doses in the treatment of chronic obstructive lung diseases in Chernobyl accident liquidators]. Ter Arkh 2002, 74(3):62-65.
341. Winklhofer-Roob B M: Cystic fibrosis: nutritional status and micronutrients [In Process Citation]. Curr Opin Clin Nutr Metab Care 2000, 3(4):293-297.
342. Shapiro B L, Smith Q T, Warwick W J: Red cell glutathione and glutathione reductase in cystic fibrosis. Proc Soc Exp Biol Med 1973, 144(1): 181-183.
343. Shapiro B L, Smith Q T, Warick W J: Serum glutathione reductase and cystic fibrosis. Pediatr Res 1975, 9(12):885-888.
344. Roum J H, Buhl R, Mcelvaney N G, Borok Z, Crystal R G: Systemic deficiency of glutathione in cystic fibrosis. Journal of Applied Physiology 1993, 75(6):2419-2424.
345. Mangione S, Patel D D, Levin B R, Fiel S B: Erythrocytic glutathione in cystic fibrosis. A possible marker of pulmonary dysfunction. Chest 1994, 105(5):1470-1473.
346. Lands L C, Grey V L, Grenier C: Total plasma antioxidant capacity in cystic fibrosis. Pediatr Pulmonol 2000, 29(2):81-87.
347. Cantin A M, R. C. H, Crystal R G: Glutathione deficiency in the epithelial lining fluid of the lower respiratory tract in idiopathic pulmonary fibrosis. Am Rev Respir Dis 1989, 139:370-372.
348. Rahman Q, Abidi P, Afaq F, Schiffmnn D, Mossman B T, Kamp D W, Athar M: Glutathione redox system in oxidative lung injury. Critical Reviews in Toxicology 1999, 29(6):543-568.
349. Meyer A, Buhl R, Magnussen H: The effect of oral N-Acetylcysteine on lung glutathione levels in idiopathic pulmonary fibrosis. European Respiratory Journal 1994, 7(3):431-436.
350. Meyer A, Buhl R, Kampf S, Magnussen H: Intravenous N-acetylcysteine and lung glutathione of patients with pulmonary fibrosis and normals. American Journal of Respiratory and Critical Care Medicine 1995, 152(3):1055-1060.
351. Rahman I, Skwarska E, Henry M, Davis M, O'Connor C M, FitzGerald M X, Greening A, MacNee W: Systemic and pulmonary oxidative stress in idiopathic pulmonary fibrosis. Free Radical Biology and Medicine 1999, 27(1-2):60-68.
352. Roum J H, Borok Z, McElvaney N G, Grimes G J, Bokser A D, Buhl R, Crystal R G: Glutathione aerosol suppresses lung epithelial surface inflammatory cell-derived oxidants in cystic fibrosis. Journal of Applied Physiology 1999, 87(1):438-443.
353. Sprince H, Parker C M, Smith G G, Gonzales L J: Protective action of ascorbic acid and sulfur compounds against acetaldehyde toxicity: implications in alcoholism and smoking. Agents Actions 1975, 5(2):164-173.
354. Michelet F, Gueguen R, Leroy P, Wellman M, Nicolas A, Siest G: Blood and plasma glutathione measured in healthy subjects by HPLC: relation to sex, aging, biological variables, and life habits. Clinical Chemistry 1995, 41(10):1509-1517.
355. Pendyala L, Schwartz G, Bolanowska-Higdon W, Hitt S, Zdanowicz J, Murphy M, Lawrence D, Creaven P J: Phase I/pharmacodynamic study of N-acetylcysteine/oltipraz in smokers: early termination due to excessive toxicity. Cancer Epidemiol Biomarkers Prev 2001, 10(3):269-272.
356. Sprince H: Protective action of sulfur compounds against aldehyde toxicants of cigarette smoke. Eur J Respir Dis Suppl 1985, 139:102-112.
357. Schmidinger M, Budinsky A C, Wenzel C, Piribauer M, Brix R, Kautzky M, Oder W, Locker G J, Zielinski C C, Steger G G: Glutathione in the prevention of cisplatin induced toxicities. A prospectively randomized pilot trial in patients with head and neck cancer and non small cell lung cancer. Wien Klin Wochenschr 2000, 112(14):617-623.
358. Hammarqvist F, Luo J L, Cotgreave I A, Andersson K, Wernerman J: Skeletal muscle glutathione is depleted in critically ill patients. Crit Care Med 1997, 25(1):78-84.
359. Kretzschmar M, Pfeiffer L, Schmidt C, Schimmeister W: Plasma levels of glutathione, alpha-tocopherol and lipid peroxides in polytraumatized patients; evidence for a stimulating effect of TNF alpha on glutathione synthesis. Exp Toxicol Pathol 1998, 50(4-6):477-483.
360. Kiefer P, Vogt J, Radermacher P: From mucolytic to antioxidant and liver protection: new aspects in the intensive care unit career of N-acetylcysteine. Crit Care Med 2000, 28(12):3935-3936.
361. Henderson A, Hayes P: Acetylcysteine as a cytoprotective antioxidant in patients with severe sepsis: potential new use for an old drug. Ann Pharmacother 1994, 28(9):1086-1088.
362. Weikert L F, Bernard G R: Pharmacotherapy of sepsis. Clin Chest Med 1996, 17(2):289-305.
363. Ortolani O, Conti A, De Gaudio A R, Moraldi E, Novelli G P: [Glutathione and N-acetylcysteine in the prevention of free-radical damage in the initial phase of septic shock]. Recenti Prog Med 2002, 93(2):125-129.
364. Pacht E R, Timerman A P, Lykens M G, Merola A J: Deficiency of alveolar fluid glutathione in patients with sepsis and the adult respiratory distress syndrome. Chest 1991, 100(5):1397-1403.
365. Pena L R, Hill D B, McClain C J: Treatment with glutathione precursor decreases cytokine activity. J Parenter Enteral Nutr 1999, 23(1):1-6.
366. Verjee Z H, Behal R: Protein-calorie malnutrition: a study of red blood cell and serum enzymes during and after crisis. Clin Chim Acta 1976, 70(1):139-147.
367. Repetto M, Reides C, Carretero M L G, Costa M, Griemberg G, Llesuy S: Oxidative stress in blood of HIV infected patients. Clinica ChimicaActa 1996, 255(2):107-117.
368. Sulkowski M S, Thomas D L, Chaisson R E, Moore R D: Hepatotoxicity associated with antiretroviral therapy in adults infected with human immunodeficiency virus and the role of hepatitis C or B virus infection. Jama 2000, 283(1):74-80.
369. Gisolf E H, Dreezen C, Danner S A, Weel J L, Weverling G J: Risk factors for hepatotoxicity in HIV-1-infected patients receiving ritonavir and saquinavir with or without stavudine. Prometheus Study Group. Clin Infect Dis 2000, 31(5):1234-1239.
370. Droge W, Eck H P, Naher H, Pekar U, Daniel V: Abnormal amino-acid concentrations in the blood of patients with acquired immunodeficiency syndrome (AIDS) may contribute to the immunological defect. Biol Chem Hoppe Seyler 1988, 369(3): 143-148.
371. Droge W, Eck H P, Mihm S: HIV-induced cysteine deficiency and T-cell dysfunction-a rationale for treatment with N-acetylcysteine. Immunol Today 1992, 13(6):211-214.
372. Buhl R, Holroyd K J, Mastrangeli A, Cantin A M, Jaffe H A, Wells F B, Saltini C, Crystal R G: Systemic glutathione deficiency in symptom-free HIV-seropositive individuals. Lancet 1989, ii(December 2):1294-1298.
373. Staal F J, Roederer M, Herzenberg L A: Intracellular thiols regulate activation of nuclear factor kappa B and transcription of human immunodeficiency virus. Proc Natl Acad Sci USA 1990, 87(24):9943-9947.
374. Staal F J, Ela S W, Roederer M, Anderson M T, Herzenberg L A: Glutathione deficiency and human immunodeficiency virus infection. Lancet 1992, 339(8798):909-912.
375. Roederer M, Raju P A, Staal F J, Herzenberg L A: N-acetylcysteine inhibits latent HIV expression in chronically infected cells. AIDS Research & Human Retroviruses 1991, 7(6):563-567.
376. Roederer M, Staal F J T, Osada H, Herzenberg L A, Herzenberg L A: CD4 and CD8 T cells with high intracellular glutathione levels are selectively lost as the HIV infection progresses. Intl Immunol 1991, 3(9):933-937.
377. Eylar E, Rivera-Quinones C, Molina C, Baez I, Molina F, Mercado C M: N-acetylcysteine enhances T cell functions and T cell growth in culture. Int Immunol 1993, 5(1):97-101.
378. Cayota A, Vuillier F, Gonzalez G, Dighiero G: CD4+ lymphocytes from HIV-infected patients display impaired CD45-associated tyrosine phosphatase activity which is enhanced by anti-oxidants. Clin Exp Immunol 1996, 104(1):11-17.
379. de Quay B, Malinverni R, Lauterburg B H: Glutathione depletion in HIV-infected patients: role of cysteine deficiency and effect of oral N-acetylcysteine. Aids 1992, 6(8):815-819.
380. Staal F J, Roederer M, Israelski D M, Bubp J, Mole L A, McShane D, Deresinski S C, Ross W, Sussman H, Raju P A et al: Intracellular glutathione levels in T cell subsets decrease in HIV-infected individuals. AIDS Res Hum Retroviruses 1992, 8(2):305-311.
381. Holroyd K J, Buhl R, Borok Z, Roum J H, Bokser A D, Grimes G J, Czerski D, Cantin A M, Crystal R G: Correction of glutathione deficiency in the lower respiratory tract of HIV seropositive individuals by glutathione aerosol treatment. Thorax 1993, 48(10):985-989.
382. Smith C V, Rogers L K, Rabin R L, Maldonado Y A, Herzenberg L A, Herzenberg L A, Petru A: Effects of human immunodeficiency virus (HIV) infection on plasma glutathione status in children. Pediatr Res 1994, 35:196A (Abstract #1163).
383. Jeannin P, Delneste Y, Lecoanet-Henchoz S, Gauchat J-F, Life P, Holmes D, Bonnefoy J-Y: Thiols decrease human interleukin (IL) 4 production and IL-4-induced immunoglobulin synthesis. Journal of Experimental Medicine 1995, 182(6): 1785-1792.
384. Helbling B, von Overbeck J, Lauterburg B H: Decreased release of glutathione into the systemic circulation of patients with HIV infection. Eur J Clin Invest 1996, 26(1):38-44.
385. Skurnick J H, Bogden J D, Baker H, Kemp F W, Sheffet A, Quattrone G, Louria D B: Micronutrient profiles in HIV-1-infected heterosexual adults. JAcq Immun Defic Synd Hum R 1996, 12(1):75-83.
386. Pacht E R, Diaz P, Clanton T, Hart J, Gadek J E: Alveolar fluid glutathione decreases in asymptomatic HIV-seropositive subjects over time. Chest 1997, 112(3):785-788.
387. Walmsley S L, Winn L M, Harrison M L, Uetrecht J P, Wells P G: Oxidative stress and thiol depletion in plasma and peripheral blood lymphocytes from HIV-infected patients: toxicological and pathological implications. AIDS 1997, 11 (14):1689-1697.
388. Jahoor F, Jackson A, Gazzard B, Philips G, Sharpstone D, Frazer M E, Heird W: Erythrocyte glutathione deficiency in symptom-free HIV infection is associated with decreased synthesis rate. Am J Physiol 1999, 276(1 Pt 1):E205-211.
389. Kim H, Lim J W, Seo J Y, Kim K H: Oxidant-sensitive transcription factor and cyclooxygenase-2 by Helicobacter pylori stimulation in human gastric cancer cells. J Environ Pathol Toxicol Oncol 2002, 21(2):121-129.
390. Verhulst M-L, Van Oijen A H A M, Roelofs H M J, Peters W H M, Jansen J B M J: Antral glutathione concentration and glutathione S-transferase activity in patients with and without Helicobacter pylori. Digestive Diseases and Sciences 2000, 45(3):629-632.
391. Boon A C, Vos A P, Graus Y M, Rimmelzwaan G F, Osterhaus A D: In vitro effect of bioactive compounds on influenza virus specific B- and T-cell responses. Scand J Immunol 2002, 55(1):24-32.
392. Ben-Menachem E, Kyllerman M, Marklund S: Superoxide dismutase and glutathione peroxidase function in progressive myoclonus epilepsies. Epilepsy Research 2000, 40(1):33-39.
393. Hurd R W, Wilder B J, Helveston W R, Uthman B M: Treatment of four siblings with progressive myoclonus epilepsy of the Unverricht-Lundborg type with N-acetylcysteine. Neurology 1996, 47(5):1264-1268.
394. Sung L, Simons J A, Dayneka N L: Dilution of intravenous N-acetylcysteine as a cause of hyponatremia. Pediatrics 1997, 100(3 Pt 1):389-391.
395. Selwa L M: N-acetylcysteine therapy for Unverricht-Lundborg disease. Neurology 1999, 52(2):426-427.
396. Sido B, Hack V, Hochlehnert A, Lipps H, Herfarth C, Droge W: Impairment of intestinal glutathione synthesis in patients with inflammatory bowel disease. Gut 1998, 42(4):485-492.
397. lantomasi T, Marraccini P, Favilli F, Vincenzini M T, Ferretti P, Tonelli F: Glutathione metabolism in Crohn's disease. Biochemical Medicine and Metabolic Biology 1994, 53(2):87-91.
398. Ruan E A, Rao S, Burdick J S, Stryker S J, Telford G L, Otterson M F, Opara E C, Koch T R: Glutathione levels in chronic inflammatory disorders of the human colon. Nutrition Research 1997, 17(3):463-473.
399. Miralles-Barrachina O, Savoye G, Belmonte-Zalar L, Hochain P, Ducrotte P, Hecketsweiler B, Lerebours E, Dechelotte P: Low levels of glutathione in endoscopic biopsies of patients with Crohn's colitis: the role of malnutrition. Clinical Nutrition 1999, 18(5):313-317.
400. Peters W H, Roelofs H M, Hectors M P, Nagengast F M, Jansen J B: Glutathione and glutathione S-transferases in Barrett's epithelium. Br J Cancer 1993, 67(6):1413-1417.
401. Mutimer D, Neuberger J: Acute liver failure: improving outcome despite a paucity of treatment options. Q J Med 1993, 86(7):409-411.
402. Fontana R J, McCashland T M, Benner K G, Appelman H D, Gunartanam N T, Wisecarver J L, Rabkin J M, Lee W M: Acute liver failure associated with prolonged use of bromfenac leading to liver transplantation. The Acute Liver Failure Study Group. Liver Transpl Surg 1999, 5(6):480-484.
403. Montanini S, Sinardi D, Pratico C, Sinardi A U, Trimarchi G: Use of acetylcysteine as the life-saving antidote in Amanita phalloides (Death cap) poisoning—Case report on 11 patients. Arzneimittel—Forschung—Drug Research 1999, 49(12): 1044-1047.
404. Ben-Ari Z, Vaknin H, Tur-Kaspa R: N-acetylcysteine in acute hepatic failure (Non-paracetamol-induced). Hepato—Gastroenterology 2000, 47(33):786-789.
405. Angulo P, Lindor K D: Treatment of nonalcoholic fatty liver: present and emerging therapies. Semin Liver Dis 2001, 21(1):81-88.
406. Altomare E, Vendemiale G, Albano 0: Hepatic glutathione content in patients with alcoholic and non alcoholic liver disease. Life Sci 1988, 43:991-998.
407. Bianchi G, Bugianesi E, Ronchi M, Fabbri A, Zoli M, Marchesini G: Glutathione kinetics in normal man and in patients with liver cirrhosis. Journal of Hepatology 1997, 26(3):606-613.
408. Tatebe S, Sinicrope F A, Kuo M T: Induction of multidrug resistance proteins MRP1 and MRP3 and gamma-glutamylcysteine synthetase gene expression by nonsteroidal anti-inflammatory drugs in human colon cancer cells. Biochem Biophys Res Commun 2002, 290(5):1427-1433.
409. Gotoh Y, Noda T, Iwakiri R, Fujimoto K, Rhoads C A, Aw T Y: Lipid peroxide-induced redox imbalance differentially mediates CaCo-2 cell proliferation and growth arrest. Cell Prolif 2002, 35(4):221-235.
410. Guan X, Hoffman B N, McFarland D C, Gilkerson K K, Dwivedi C, Erickson A K, Bebensee S, Pellegrini J: Glutathione and mercapturic acid conjugates of sulofenur and their activity against a human colon cancer cell line. Drug Metab Dispos 2002, 30(3):331-335.
411. Nakagawa Y, Akao Y, Morikawa H, Hirata I, Katsu K, Naoe T, Ohishi N, Yagi K: Arsenic trioxide-induced apoptosis through oxidative stress in cells of colon cancer cell lines. Life Sci 2002, 70(19):2253-2269.
412. Lou M F, Dickerson Jr J E, Tung W H, Wolfe J K, Chylack Jr L T: Correlation of nuclear color and opalescence with protein S-thiolation in human lenses. Experimental Eye Research 1999, 68(5):547-552.
413. Chasan-Taber L, Willett W C, Seddon J M, Stampfer M J, Rosner B, Colditz G A, Speizer F E, Hankinson S E: A prospective study of carotenoid and vitamin A intakes and risk of cataract extraction in US women. Am J Clin Nutr 1999, 70(4):509-516.
414. Brown L, Rimm E B, Seddon J M, Giovannucci E L, Chasan-Taber L, Spiegelman D, Willett W C, Hankinson S E: A prospective study of carotenoid intake and risk of cataract extraction in US men. Am J Clin Nutr 1999, 70(4):517-524.
415. Saxena S, Kumar D, Srivastava P, Khanna V K, Seth P K: Low levels of platelet glutathione in Eales' disease. Medical Science Research 1999, 27(9):625-626.
416. Tenenbein M: Hypersensitivity-like reactions to N-acetylcysteine. Vet Hum Toxicol 1984, 26(Suppl 2):3-5.
417. Kokcam I, Naziroglu M: Antioxidants and lipid peroxidation status in the blood of patients with psoriasis. Clinica Chimica Acta 1999, 289(1-2):23-31.
418. Bijlmer-lest J C, Baart de la Faille H, van Asbeck B S, van Hattum J, van Weelden H, Marx J J, Koningsberger J C: Protoporphyrin photosensitivity cannot be attenuated by oral N-acetylcysteine. Photodermatol Photoimmunol Photomed 1992, 9(6):245-249.
419. Hansen R M, Csuka M E, McCarty D J, Saryan L A: Gold induced aplastic anemia. Complete response to corticosteroids, plasmapheresis, and N-acetylcysteine infusion. J Rheumatol 1985, 12(4):794-797.
420. Maurice M M, Nakamura H, van der Voort E A M, van Viet Al, Staal F J T, Tak P-P, Breedveld F C, Verweij C L: Evidence for the role of an altered redox state in hyporesponsiveness of synovial T cells in rheumatoid arthritis. J Immunol 1997, 158(3): 1458-1465.
421. Gringhuis S I, Leow A, Papendrecht-van der Voort E A M, Remans P H J, Breedveld F C, Verweij C L: Displacement of linker for activation of T cells from the plasma membrane due to redox balance alterations results in hyporesponsiveness of synovial fluid T lymphocytes in rheumatoid arthritis. Journal of Immunology 2000, 164(4):2170-2179.
422. Aukrust P, Svardal A M, Muller F, Lunden B, Berge R K, Froland S S: Decreased levels of total and reduced glutathione in CD4+ lymphocytes in common variable immunodeficiency are associated with activation of the tumor necrosis factor system: possible immunopathogenic role of oxidative stress. Blood 1995, 86(4): 1383-1391.
423. Giovannucci E, Rimm E B, Stampfer M J, Colditz G A, Willett W C: Diabetes mellitus and risk of prostate cancer (United States). Cancer Causes Control 1998, 9(1):3-9.
424. Chiao J W, Chung F L, Kancherla R, Ahmed T, Mittelman A, Conaway C C: Sulforaphane and its metabolite mediate growth arrest and apoptosis in human prostate cancer cells. Int J Oncol 2002, 20(3):631-636.
425. Hursting S D, Shen J C, Sun X Y, Wang T T, Phang J M, Perkins S N: Modulation of cyclophilin gene expression by N-4-(hydroxyphenyl)retinamide: association with reactive oxygen species generation and apoptosis. Mol Carcinog 2002, 33(1):16-24.
426. Davidson S D, Milanesa D M, Mallouh C, Choudhury M S, Tazaki H, Konno S: A possible regulatory role of glyoxalase I in cell viability of human prostate cancer. Urol Res 2002, 30(2):116-121.
427. Bounous G: Whey protein concentrate (WPC) and glutathione modulation in cancer treatment. Anticancer Res 2000, 20(6C):4785-4792.
428. Xu D, Finkel T: A role for mitochondria as potential regulators of cellular life span. Biochem Biophys Res Commun 2002, 294(2):245-248.
429. Shan X, Aw T Y, Jones D P: Glutathione-dependent protection against oxidative injury. Pharmacol Ther 1990, 47:61-71.
430. Ponnappan U: Ubiquitin-proteasome pathway is compromised in CD45RO+ and CD45RA+ T lymphocyte subsets during aging. Exp Gerontol 2002, 37(2-3):359-367.
431. Lang C A, Naryshkin S, Schnieder D L, Mills B J, Lindeman R D: Low blood glutathione in healthy aging adults. J Lab Clin Med 1992, 120(5):720-725.
432. Julius M, Lang C A, Gleiberman L, Harburg E, DiFranceisco W, Schork A: Glutathione and morbidity in a community-based sample of elderly. Journal of Clinical Epidemiology 1994, 47(9):1021-1026.
433. Sastre J, Pallardo F V, Vina J: Glutathione, oxidative stress and aging. Age 1996, 19(4):129-139.
434. Samiec P S, Drews-Botsch C, Flagg E W, Kurtz J C, Sternberg P, Reed R L, Jones D P: Glutathione in human plasma: Decline in association with aging, age-related macular degeneration, and diabetes. Free Radical Biology and Medicine 1998, 24(5):699-704.
435. Nuttall S L, Dunne F, Kendall M J, Martin U: Age-independent oxidative stress in elderly patients with non-insulin-dependent diabetes mellitus. Qjm—Monthly Journal of the Association of Physicians 1999, 92(1):33-38.
436. Sastre J, Pallardo F V, Garcia de la Asuncion J, Vina J: Mitochondria, oxidative stress and aging. Free Radic Res 2000, 32(3):189-198.
437. Martin D S, Willis S E, Cline D M: N-acetylcysteine in the treatment of human arsenic poisoning. J Am Board Fam Pract 1990, 3(4):293-296.
438. Whitekus M J, Li N, Zhang M, Wang M, Horwitz M A, Nelson S K, Horwitz L D, Brechun N, Diaz-Sanchez D, Nel A E: Thiol antioxidants inhibit the adjuvant effects of aerosolized diesel exhaust particles in a murine model for ovalbumin sensitization. J Immunol 2002, 168(5):2560-2567.
439. Banerjee B D, Seth V, Bhattacharya A, Pasha S T, Chakraborty A K: Biochemical effects of some pesticides on lipid peroxidation and free-radical scavengers. Toxicology Letters 1999, 107(1-3):33-47.
440. Hultberg B, Andersson A, Isaksson A: Interaction of metals and thiols in cell damage and glutathione distribution: potentiation of mercury toxicity by dithiothreitol. Toxicology 2001, 156(2-3):93-100.
441. Winterboum C C, Peskin A V, Parsons-Mair H N: Thiol oxidase activity of copper, zinc superoxide dismutase. J Biol Chem 2002, 277(3):1906-1911.
442. Knapen M F C M, Mulder T P J, Van Rooij I A L M, Peters W H M, Steegers E A P: Low whole blood glutathione levels in pregnancies complicated by preeclampsia or the hemolysis, elevated liver enzymes, low platelets syndrome. Obstetrics and Gynecology 1998, 92(6):1012-1015.
443. Mutlu-Turkoglu U, Ademoglu E, Ibrahimoglu L, Aykac-Toker G, Uysal M: Imbalance between lipid peroxidation and antioxidant status in preeclampsia. Gynecologic and Obstetric Investigation 1998, 46(1):37-40.
444. Smith C V, Hansen T N, Martin N E, McMicken H W, Elliott S J: Oxidant stress responses in premature infants during exposure to hyperoxia. Pediatr Res 1993, 34(3):360-365.
445. Saugstad O D: Bronchopulmonary dysplasia and oxidative stress: are we closer to an understanding of the pathogenesis of BPD? Acta Paediatrica 1997, 86(12):1277-1282.
446. Seema, Kumar R, Mandal R N, Tandon A, Randhawa V S, Mehta G, Batra S, Ray G N, Kapoor A K: Serum TNF-alpha and free radical scavengers in neonatal septicemia. Indian J Pediatr 1999, 66(4):511-516.
447. van Bakel M M, Printzen G, Wermuth B, Wiesmann U N: Antioxidant and thyroid hormone status in selenium-deficient phenylketonuric and hyperphenylalaninemic patients. Am J Clin Nutr 2000, 72(4):976-981.
448. Krenzelok E P: New developments in the therapy of intoxications. Toxicol Lett 2002, 127(1-3):299-305.
449. Brok J, Buckley N, Gluud C: Interventions for paracetamol (acetaminophen) overdoses. Cochrane Database Syst Rev 2002(3):CD003328.
450. Listed N A: N-acetylcysteine. Altern Med Rev 2000, 5(5):467-471.
451. Sochman J: N-acetylcysteine in acute cardiology: 10 years later: what do we know and what would we like to know? J Am Coll Cardiol 2002, 39(9): 1422-1428.
452. Bains J S, Shaw C A: Neurodegenerative disorders in humans: the role of glutathione in oxidative stress-mediated neuronal death. Brain Research Reviews 1997, 25(3):335-358.
453. Banaclocha M M: Therapeutic potential of N-acetylcysteine in age-related mitochondrial neurodegenerative diseases. Med Hypotheses 2001, 56(4):472-477.
454. Reid M, Jahoor F: Glutathione in disease. Curr Opin Clin Nutr Metab Care 2001, 4(1):65-71.
   Table 1
   NAC Treatment in Randomzed Placebo-Controlled Studies

Tables entries all refer to randomized placebo-controlled studies in which NAC, or in some cases another GSH-replenishing drug, were administered. Dosage shown is for NAC unless otherwise indicated. The treatment effect is scored as “Beneficial” when reported outcome(s) differs significantly from the placebo group (p<0.05) for a clinical parameter of importance to patient well-being in the disease under study. If a significant difference between the NAC and placebo group was observed by the clinical relevance of the finding is unclear, the treatment effect is scored as “Significant (? Clin rel)”. Failure to find a significant difference is scored as “Not significant” while significant negative effects of NAC treatment are scored as “Adverse”.

TABLE 1a
Hepatic and GI pathophysiology

| NAC use in hepatic and GI | | Study | | Treatment |
|---|---|---|---|---|
| pathophysiology | n | length | Dosage | effect |
| Acetaminophen Toxicity*. IV NAC | 50 | 21 days | 150 mg/kg | Beneficial |
| administration to subjects with | | | for 15 min; | |
| acetaminophen-induced fulminant | | | 50 mg/kg | |
| hepatic failure who had not previously | | | for 4 h; | |
| received NAC (late presentation) | | | 100 mg/kg | |
| increased survival and decreased both | | | over 16 h. | |
| the incidence of cerebral edema and | | | | |
| the frequency of hypotension requiring | | | | |
| inotropic support. Rates of | | | | |
| deterioration and recovery of liver | | | | |
| function were not affected. Keays, | | | | |
| 1991[2] | | | | |
| Hepatic transplant patients. NAC | 50 | 5 hours | 150 mg/kg | Beneficial |
| induces mild vasodilation, improves | | | for 15 min; | |
| oxygen delivery and consumption, and | | | 12.5 mg/kg | |
| reduces base deficit. Bromley | | | for 4 h. | |
| 1995[19] | | | | |
| Hepatitis C. Treatment with NAC in | 147 | 1 year | 1800 mg NAC | not |
| addition to conventional therapy with | | | daily with | significant |
| interferon--□ did not result in | | | Interferon-□ | |
| significant improvement of the | | | | |
| treatment group. Grant 2000[265] | | | | |
| Colon cancer. NAC lowered the | 64 | 12 weeks | 800 mg NAC | Beneficial |
| proliferative index in the colonic crypt | | | daily, po | |
| epithelium of human volunteers who | | | | |
| previously had adenomatous polyps. | | | | |
| Estensen, 1999[55] | | | | |
| NAC safety. Gastroduodenoscopic | 20 | 7 days | 200 mg 3× | Safe (no |
| examination following oral NAC | | | daily | effect) |
| administration disclosed no lesions. | | | | |
| Histological examination of biopsy | | | | |
| specimens from the antrum and corpus | | | | |
| of the stomach disclosed no | | | | |
| pathological changes. Marini, | | | | |
| 1980[62] | | | | |
| Hyperoxic ventilation. NAC | 38 | 72 hours | 150 ml/kg IV | Beneficial |
| preserved whole body oxygen uptake, | | | over a period | |
| oxygen extraction ratio, and gastric | | | of 15 min | |
| intramucosal pH during brief | | | | |
| hyperoxic ventilation. Reinhart, | | | | |
| 1995[266] | | | | |
| Gastric mucosa. The systemic effect | 100 | 3 days | 600 mg daily | not |
| of NAC on gastric mucus was not | | prior to | | significant |
| powerful enough to improve the | | treatment | | |
| barium coating of the gastric mucosa. | | | | |
| Kinnunen, 1989[267] | | | | |
| Helicobacter Pylori. NAC decreased | 107 | 5 ml of | 107 | Interferes |
| the sensitivity and specificity of the | | 4% NAC | | with |
| Helicobacter pylori stool antigen test. | | TID for | | traditional |
| Demiturk, 2003[268] | | 3 days | | clinical |
| | | | | assay |
| Protein-energy malnutrition. | 32 | 50 days | 0.5 mmol per | Beneficial |
| Malnourished children admitted for | | | kg per day | |
| treatment of infection were treated | | | | |
| with NAC or placebo (alanine). NAC | | | | |
| restored GSH synthesis rate and | | | | |
| concentration when administered | | | | |
| during the early phase of treatment | | | | |
| Badaloo, 2002[54] | | | | |

*Note:
Although NAC is the accepted treatment for acetaminophen overdose, there do not appear to be any placebo-controlled trials that supported its initial acceptance. A series of studies that are not placebo-controlled, e.g., Smilkstein et al¹²⁷, have served this purpose (see the citations for acetaminophen under Toxic Agents in Table 2).

TABLE 1b
NAC use in cardiovascular pathophysiology

| | | Study | | Beneficial |
|---|---|---|---|---|
| | n | length | Dosage | effects |
| Hyperoxic ventilation in cardiac risk | 30 | 30 min | 150 mg/kg, IV | Beneficial |
| patients. NAC helped preserve whole- | | | | |
| body oxygen consumption (VO2), oxygen | | | | |
| delivery, cardiac index, left ventricular | | | | |
| stroke work index and venoarterial carbon | | | | |
| dioxide gradient during brief hyperoxia. | | | | |
| Clinical signs of myocardial ischemia such | | | | |
| as ST-depression did not occur if patients | | | | |
| were prophylactically treated with NAC. | | | | |
| Spies, 1996[11] | | | | |
| Stable angina and nitrate tolerance. | 10 | 24 hours | 4800 mg, po | Beneficial |
| None of the patients had developed nitrate | | | | |
| tolerance at inclusion. NAC in combination | | | | |
| with the long-acting nitrate, isosorbide-5- | | | | |
| mononitrate (5-ISMN) significantly | | | | |
| prolonged the total exercise time as | | | | |
| compared with placebo/5-ISMN. No | | | | |
| further effect was obtained after additional | | | | |
| NTG doses. Svendsen, 1989[12] | | | | |
| Stable angina and nitrate tolerance. The | 7 | 8 days | 2400 mg daily, | Beneficial |
| reduction in ST segment depression was | | | po | |
| significantly more pronounced during | | | | |
| isosorbide dinitrate (ISDN) + NAC as | | | | |
| compared with ISDN + placebo. Exercise | | | | |
| time and time to angina pectoris were | | | | |
| unaffected. NAC augmented the | | | | |
| antischemic effects of ISDN as assessed by | | | | |
| ECG. Boesgaard, 1991[13] | | | | |
| Stable angina and nitrate tolerance. | 10 | 30 hours | 2 g IV bolus | Beneficial |
| Infusion of high doses of NAC in | | | followed by | |
| combination with isosorbide dinitrate | | | 5 g/kg/hr IV | |
| (ISDN) affects and partially prevents the | | | | |
| development of tolerance to antianginal | | | | |
| effects normally observed during infusion | | | | |
| with ISDN. Boesgaard, 1992[14] | | | | |
| Unstable angina. Combined | 46 | 6 hours | 5 g, IV | Beneficial |
| administration of nitroglycerin (NTG) and | | | | |
| NAC in patients with unstable angina | | | | |
| pectoris augments the clinical efficacy of | | | | |
| NTG, largely by reducing the incidence of | | | | |
| acute myocardial infarction. However, the | | | | |
| high incidence of severe hypotension with | | | | |
| NTG/NAC suggests that this regimen | | | | |
| should be used with some caution. | | | | |
| Horowitz, 1988[175, 176] | | | | |
| Unstable angina. The combination of | 200 | 4 months | 600 mg, po | Beneficial |
| nitroglycerin and NAC, associated with | | | | |
| conventional medical therapy reduces the | | | | |
| occurrence of outcome events. The high | | | | |
| incidence of side effects (mainly | | | | |
| intolerable headaches) limits the clinical | | | | |
| applicability of this therapeutic strategy at | | | | |
| least at the dosage used in the present | | | | |
| study. Ardissino, 1997[177] | | | | |
| Unstable angina and nitrate tolerance. | 19 | 72 hours | 10 g q 24 h, | not |
| Blood pressure and the response to the | | | IV | significant |
| glyceryl trinitrate bolus we note | | | | |
| significantly affected by the co- | | | | |
| administration of intravenous NAC with | | | | |
| intravenous isosorbide trinitrate (ISDN). | | | | |
| Weber, 1992[269] | | | | |
| Atherosclerosis. Oral L-2-oxothiazolidine- | 48 | 4 hours | 4.5 g OTC, po | Beneficial |
| 4-carboxylic acid (OTC) improves brachial | | | | |
| artery flow-mediated dilation and | | | | |
| endothelium-derived nitric oxide action in | | | | |
| human atherosclerosis. Vita, 1998[15] | | | | |
| Cardiac bypass. The oxidative burst | 12 | 24 hours | 100 mg/kg | Beneficial |
| response of peripheral neutrophils in the | | | bolus; then | |
| patients receiving NAC was significantly | | | of 20 mg/kg | |
| lower at all times during bypass. Andersen | | | continuous | |
| 1995[16] | | | IV | |
| Cardiac Bypass. Data shows that | 36 | 36 hours ? | 100 mg/kg | NEED |
| cardioplegic arrest initiates the apoptosis | | | followed | Paper |
| signal cascade in human left ventricalr car- | | | by infusion | |
| diac myocytes. This apoptosis induction | | | 20 mg/kg. | |
| can effectively ne prevented by NAC. | | | | |
| Fischer, 2004. [270] | | | | |
| Myocardial ischemia. 24-hour | 96 | 24 hours | GSH, 1800 mg | Beneficial |
| intravenous infusion of GSH in addition to | | | IV bolus; | |
| thrombolytic therapy significantly | | | 20□g/kg | |
| decreased plasma MDA, an oxidative | | | per min for | |
| stress indicator that is significantly | | | 24 h. | |
| elevated by thrombolytic treatment for | | | | |
| acute myocardial infarction. Altomare, | | | | |
| 1996[179] | | | | |

TABLE 1c
NAC use in renal pathophysiology

| | | Study | | Beneficial |
|---|---|---|---|---|
| NAC use in renal pathophysiology | n | length | Dosage | effects |
| Contrast-induced nephropathy (CIN). | 83 | 2 days | 600 mg PO bid | Beneficial |
| Prophylactic oral administration of NAC | | | | |
| along with hydration in patients with | | | | |
| chronic renal insufficiency prevents the | | | | |
| reduction in renal function induced by | | | | |
| iopromide, a nonionic low-osmolality | | | | |
| contrast agent (relative risk, RR 0.10) | | | | |
| Tepel, 2000, 2001 and 2003[3-5] | | | | |
| CIN. NAC protects patients with | 200 | 3 days | 600 mg PO bid | Beneficial |
| moderate chronic renal insufficiency | | | | |
| from contrast-induced deterioration in | | | | |
| renal function after coronary | | | | |
| angiographic procedures, with minimal | | | | |
| adverse effects (RR 0.32). Kay, 2003[6] | | | | |
| CIN. NAC decreased the incidence of | 54 | 2 days | 600 mg PO bid | Beneficial |
| post-catheterization nephropathy (RR | | | | |
| 0.21) in patients with stable chronic renal | | | | |
| insufficiency undergoing elective cardiac | | | | |
| catheterization. Diaz-Sandoval, 2002[7] | | | | |
| CIN. NAC decreased the incidence of | 121 | 7 days | 400 mg PO bid | Beneficial |
| contrast-induced nephropathy (RR 0.13) | | | for 2 days | |
| in patients with stable chronic renal | | | | |
| insufficiency undergoing coronary | | | | |
| angiography. Shyu, 2002[8] | | | | |
| CIN. In patients with serum creatinine > | 49 | 4 days | 1000 mg daily | Beneficial |
| 1.2 mg/dL (106 μmol/L) undergoing | | | | |
| elective coronary angiography, NAC was | | | | |
| associated with preservation of creatinine | | | | |
| clearance at 24 and 96 hours, which | | | | |
| decreased significantly in placebo-treated | | | | |
| patients (21% at 24 hours and 18% at 96 | | | | |
| hours, both P < O.005). Efarti 2003. [9] | | | | |
| CIN. NAC treatment prevented the | 43 | 3 days | 600 mg 5 doses | Beneficial |
| development of CIN by 72 hours in | | | | |
| patients with moderate renal | | | | |
| insufficiency (serum creatinine ≧ 1.5 | | | | |
| mg/dL) undergoing elective cardiac | | | | |
| catheterization (4.8% vs 32.2%, P = | | | | |
| 0.046). MacNeil 2003. [10] | | | | |
| CIN: NAC is strongly associated with | 24 | 4 days | 1200 mg daily | Beneficial |
| suppression of oxidative stress-mediated | | | | |
| proximal tubular injury. Drager, 2004. | | | | |
| [271] | | | | |
| CIN. NAC failed to prevent CIN at 24 | 108 | 1 day | 1200 mg | not |
| hours following coronary angiography in | | | | significant |
| the group as a whole. The overall | | | | |
| incidence was low (4.8%). Kefer et al. | | | | |
| [189] | | | | |
| CIN. NAC failed to prevent CIN by 48 | 96 | 2 days | 1500 mg for 4 | not |
| hours in patients with moderate chronic | | | doses | significant |
| renal insufficiency (serum creatinine > | | | | |
| 1.2 mg/dL and creatinine clearance < 50 | | | | |
| mL/min) undergoing elective coronary | | | | |
| angiography. The incidence of CIN in | | | | |
| both groups was low (NAC 8.2%, control | | | | |
| 6.4%). Oldemeyer 2003 [188] | | | | |
| CIN. In patients with reduced renal | 183 | 2 days | 600 mg PO bid | not |
| function undergoing angiography and/or | | | | significant |
| angioplasty, the amount of contrast agent, | | | | |
| but not the administration of prophylactic | | | | |
| NAC, was a predictor of renal function | | | | |
| deterioration. Brigouri, 2000[190] | | | | |
| CIN. NAC failed to protect against | 123 | 2 days | 600 mg PO bid | not |
| radiocontrast-induced nephropathy in | | | | significant |
| patients with baseline renal dysfunction | | | | |
| (serum creatinine > 1.6 mg/dL or | | | | |
| creatinine clearance < 60 mL/min) | | | | |
| undergoing “cardiovascular | | | | |
| interventions” using low-osmolality | | | | |
| nonionic contrast media. Allaqaband et | | | | |
| al, 2002[186] | | | | |
| CIN. NAC failed to protect against | 79 | 8 | 1200 mg 1 hr | not |
| radiocontrast-induced nephropathy in | | | prior and 3 hrs | significant |
| patients with baseline renal dysfunction | | | after procedure | |
| (serum creatinine > 1.7 mg/dL) | | | | |
| undergoing cardiac catheterization using | | | | |
| low-osmolality nonionic contrast media. | | | | |
| Durham, 2002[185] | | | | |
| CIN. NAC administration had no | 179 | 2 days | 600 mg 4 times | not |
| influence on the incidence of CIN at 48 | | | daily | significant |
| hours (NAC, 13%; control 12%) after | | | | |
| elective cardiac catheterization in patients | | | | |
| with a serum creatinine > 1.2 mg/dL or a | | | | |
| creatinine clearance < 50 mL/min. | | | | |
| Boccalandro 2003. [187] | | | | |
| CIN. NAC administration had no | 80 | 1 day | 600 mg PO tid | not |
| significant effect vs. placebo. | | | | significant |
| Goldenberg 2004. [272] | | | | |
| Hemodyalysis. Acetylcysteine- | 20 | | 5 g in 5% | Beneficial |
| dependant increase of homocysteine | | | glucose and | |
| removal during a hemodialysis session | | | 150 mg/kg for | |
| improves plasma homocysteine | | | nephrophy. | |
| concentration, pulse pressure and | | | | |
| endothelial function in patients withf | | | | |
| end-stage renal failure. Scholze, 2004. | | | | |
| [273] | | | | |
| Hemodialysis. Treatment of patients | 134 | 1-24 months | 600 mg bid | Beneficial |
| with NAC reduces composite | | | | |
| cardiovascular end points. Median | | | | |
| treatment time was 14.5 months. Tepel | | | | |
| 2003[17] | | | | |

TABLE 1d
NAC use in endocrine pathophysiology

| | | Study | | Beneficial |
|---|---|---|---|---|
| NAC use in endocrine pathophysiology | n | length | Dosage | effects |
| Polycystic Ovary Syndrome. NAC | 33 | 6 weeks | 1.8-3 g daily | Beneficial |
| decreases circulating insulin levels and | | | depending on | |
| decreases insulin sensitivity in patients | | | weight | |
| with polycystic ovary syndrome. Fulghesu | | | | |
| 2002[20] | | | | |
| Diabetes. Oral NAC treatment increased | 15 | 1 month | 1200 mg/day, po | Beneficial |
| erythrocyte GSH concentrations and | | M | | |
| GSH:GSSG ratio. NAC also decreased | | | | |
| plasma soluble vascular cell adhesion | | | | |
| molecule (VCAM-1), a measure of | | | | |
| vascular damage in non-insulin dependent | | | | |
| diabetes. De Mattia, 1998[21] | | | | |
| Diabetes. IV administration of GSH | 20 | 2 hours | GSH, 1.35 | Beneficial |
| increased both intra-erythrocytic | | | gm²min⁻¹ IV | |
| GSH/GSSG ratio and total glucose uptake | | | | |
| in non-insulin dependent diabetic | | | | |
| (NIDDM) patients. Significant | | | | |
| correlations were found between | | | | |
| GSH/GSSG ratio. De Mattia, 1998[22] | | | | |

TABLE 1e
NAC use in metabolic and genetic pathophysiology

| NAC use in metabolic and genetic | | Study | | Beneficial |
|---|---|---|---|---|
| pathophysiology | n | length | Dosage | effects |
| Sickle Cell Disease/Vaso-occlusive | 21 | 12 Months | 2,400 mg daily | Beneficial |
| disease. NAC inhibited dense cell | | | | |
| formation, restored glutathione levels | | | | |
| toward normal, and decreased vaso- | | | | |
| occlusive episodes (0.03 to 0.006 episodes | | | | |
| per person-days). Pace 2003[18] | | | | |
| Cystic Fibrosis (CF) and primary ciliary | 54 | 3 months | 600 mg < 30 kg | Beneficial |
| dyskinesia (PCD). No effect was seen in | | | or 800 mg > 30 kg | |
| PCD patients. However, but lung function | | | perday, po | |
| improved in NAC-treated CF patients in | | | | |
| the period when the patients suffer most | | | | |
| from lower airway infections. | | | | |
| Stafanger, 1988[40] | | | | |
| CF. NAC treatment of patients with | 52 | 3 months | 600 mg < 30 kg | Beneficial |
| chronic pulmonary Pseudomonas | | | or 800 mg > 30 kg | to a |
| aeruginosa infection did not significantly | | | per day, po | subgroup |
| improve lung function in the overall | | | | |
| treatment group. In patients with peak | | | | |
| expiratory flow rate (PEFR) below 70% of | | | | |
| predicted normal values, NAC | | | | |
| significantly increased PEFR, forced vital | | | | |
| capacity (FVC) and forced expiratory | | | | |
| volume in one second (FEV1) during the | | | | |
| treatment period. Stafanger, 1989[41] | | | | |
| CF. NAC and Ambroxol improved air | 36 | 12 weeks | NAC or | Beneficial |
| trapping and FEV1. No other clinical | | | Ambroxol | |
| differences were observed between the three | | | 200 mg 3 | |
| groups. Ratjen, 1985[274] | | | times daily | |
| CF. Although the forced expiratory | 21 | 14 days^{M} | 9.5 mg/kg/day | not |
| volume in one second (FEV1) improved in | | | po (average) | significant |
| a number of NAC-trteated patients, the | | | | |
| mean increase was within the range of the | | | | |
| intra-individual variability. Gotz, | | | | |
| 1980[275] | | | | |
| Homocysteine in hemodialysis. NAC | 20 | 1 session | Intravenous 5 g | Beneficial |
| significantly increased the removal of | | | in 5% glucose | |
| homocysteine by hemodialisys. Plasma | | | solution | |
| homocysteine levels decreased to 12 +/− 7% | | | | |
| (mean +/− SD) of predialysis level in the | | | | |
| treatment group compared with 58 +/− 22% | | | | |
| in the control group (P < 0.01). The | | | | |
| reduction of plasma homocysteine | | | | |
| concentration was significantly correlated | | | | |
| with a reduction of pulse pressure and | | | | |
| improved in endothelial function. Scholze | | | | |
| 2004[273] | | | | |
| Homocysteine in hemodialysis. Plasma | 38 | 4 weeks | 1200 mg BID | not |
| homocysteine levels decreased with | | | | significant |
| chronic oral NAC therapy (−19%) but did | | | | |
| not significantly differ from placebo- | | | | |
| treated subjects (−8%, P = 0.07). | | | | |
| Friedman, 2003 [276] | | | | |
| High plasma homocysteine. In a double | 11 | 14 days | 4000 mg daily, | Beneficial |
| blind cross over trial in patients with high | | | po | |
| plasma Lp(a) (>0.3 g/l), NAC treatment | | | | |
| had no effect on plasma lipoprotein(a) | | | | |
| levels but significantly reduced plasma | | | | |
| homocysteine. Wiklund, 1996[277] | | | | |

TABLE 1f
NAC use in Pulmonary pathophysiology

| NAC use in Pulmonary | | Study | | Beneficial |
|---|---|---|---|---|
| pathophysiology | n | length | Dosage | effects |
| Asthma. NAC medication had no effect | 25 | 9 weeks | 600 mg | Not |
| on any spirometric, lung mechanic or | | | | significant |
| gas exchange variable, nor on the | | | | |
| frequency of pulmonary symptoms. | | | | |
| Bylin 1987[23] | | | | |
| Bronchitis. The frequency of | 69 | 6 months | 600 mg/day, | Beneficial |
| exacerbations was significantly lower in | | | 3 days/wk, po | |
| the NAC-treated group. Grassi and | | | | |
| Morandini, 1976[24] | | | | |
| Bronchitis. Oral NAC changes the | 29 | 4 weeks | 600 mg/day, po | Beneficial |
| consistency of sputum with resultant | | | | |
| ease of expectoration and the | | | | |
| expectoration of increased volumes of | | | | |
| sputum; peak expiratory flow rate was | | | | |
| increased. Aylward, 1980[25] | | | | |
| Bronchitis. NAC was effective in | 215 | 10 days | 600 mg/day, po | Beneficial |
| decreasing sputum volume and viscosity | | | | |
| in patients with acute and chronic | | | | |
| bronchitis. Brocard, 1980[26] | | | | |
| Bronchitis. Oral NAC was associated | 744 | 6 months | 400 mg/day, po | Beneficial |
| with decreased thickness of sputum, | | | | |
| improved ease of expectoration, and | | | | |
| decreased incidence of exacerbations. | | | | |
| Grassi, 1980, Ferrari, 1980[27, 28] | | | | |
| Bronchitis. The exacerbation rate was | 259 | 6 months | 400 mg/day, po | Beneficial |
| significantly lower in the NAC-treated | | | | |
| group. Boman, 1983[29] | | | | |
| Bronchitis. Although improvement in | 155 | 3 months | 600 mg/day, po | Beneficial |
| subjective symptoms (sputum viscosity | | | | |
| and character, difficulty in expectoration | | | | |
| and cough severity) occurred in both | | | | |
| treatment groups, improvements in | | | | |
| difficulty in expectoration and cough | | | | |
| severity were greater in patients | | | | |
| receiving NAC. Jackson, 1984[30] | | | | |
| Bronchitis. The number of days on | 526 | 6 months | 600 mg/day, po | Beneficial |
| which subjects in the NAC group were | | | | |
| incapacitated was significantly | | | | |
| decreased. There was no statistically | | | | |
| significant difference in the number of | | | | |
| exacerbations between the NAC and | | | | |
| placebo groups although there was a | | | | |
| slight trend towards improvement in the | | | | |
| NAC group during the first 3 months. | | | | |
| Parr and Huitson, 1987[31] | | | | |
| Bronchitis. NAC-treated group had | 116 | 6 months | 600 mg/day, po | Beneficial |
| reduced number of sick-leave days | | | | |
| caused by exacerbations of chronic | | | | |
| bronchitis. Rasmussen and Glennow, | | | | |
| 1988[32] | | | | |
| Bronchitis. Pre-treatment with NAC | 37 | 22 weeks | 1200 mg/day, po | Beneficial |
| resulted in a significantly greater | | | | |
| increase in evening peak flows. Evald, | | | | |
| 1989[33] | | | | |
| Bronchitis. NAC was significantly | 153 | 22 weeks | 1200 mg/day, po | Beneficial |
| superior to placebo in terms of a | | | | |
| favourable effect on General Health | | | | |
| Questionnaire score, which measures | | | | |
| general well-being in patients with mild | | | | |
| chronic bronchitis.. Hansen, 1994[34] | | | | |
| Bronchitis. No significant result was | 59 | 2 years | 600 mg daily | not |
| found with respect to the frequency of | | | | significant |
| coughs, dyspenea, sleep disorder, | | | | |
| expectorate, morning and afternoon | | | | |
| PEF, and PEF, FEV. Heinig, 1985[200] | | | | |
| Bronchitis. There was a trend toward | 181 | 5 months | 600 mg/day, po | not |
| significantly reduced number of | | | | significant |
| exacerbations in the NAC-treated group | | | | |
| but differences did not reach | | | | |
| conventional levels of statistical | | | | |
| significance. McGavin, 1985[159] | | | | |
| Bronchitis. No significant differences | 9 | 4 weeks | 600 mg/day, po | not |
| were found in lung function, | | | | significant |
| mucociliary clearance curves or sputum | | | | |
| viscosity following treatment with NAC | | | | |
| compared to control or placebo | | | | |
| measurements. Millar, 1985[201] | | | | |
| Bronchitis. Differences between the | 161 | 5 months | 600 mg/day, po | not |
| NAC and placebo groups did not reach | | | | significant |
| conventional levels of statistical | | | | |
| significance for the number of | | | | |
| exacerbations, total days taking an | | | | |
| antibiotic; total days spent in bed, | | | | |
| number of withdrawals, incidence of | | | | |
| side effects, drug compliance, and the | | | | |
| patients' assessment of the treatment. | | | | |
| McGavin, 1985[202] | | | | |
| Bronchitis. NAC delivered by metered | 65 | 16 weeks | 4 mg twice | not |
| dose inhalers did not have any | | | daily, inhaled | significant |
| significant effect on patients' feeling of | | | | |
| well-being, sensation of dyspnea, | | | | |
| intensity of coughing, mucus | | | | |
| production, or expectoration or lung | | | | |
| function. NAC effect on exacerbations | | | | |
| could not be estimated because the | | | | |
| number of reported exacerbations was | | | | |
| too low. Dueholm, 1992[204] | | | | |
| COPD. Long-term oral administration | 44 | 12 months | 600 mg fizzy | Beneficial |
| of NAC attenuates hydrogen peroxide | | | | |
| formation in the airways of COPD | | | | |
| subjects. Kasielski, 2001[210] | | | | |
| COPD. The NAC group had | 60 | 10 days | 600 mg | Beneficial |
| significantly improved mucus viscosity, | | | | |
| effective cough and clinical symptoms. | | | | |
| Verstraeten, 1979[39] | | | | |
| COPD. The major findings of this | 9 | 15 | 1800 mg for 4 | Beneficial |
| study was that short-term, High-dose | | | days and 600 | |
| NAC treatment impoved quadriceps | | | mg the day of | |
| endurance, reduced the disturbance in | | | the test | |
| the prooxidant system and prevented | | | | |
| exercise induced oxidative stress. | | | | |
| Koechlin, 2004. [278] | | | | |
| Smoking. Improved mucus clearance in | 12 | 30 days | 600 mg/day | Beneficial |
| subjects on NAC when compared to | | | | |
| placebo. Olivieri, 1985[36] | | | | |
| Smoking. Observed an inhibitory effect | 41 | 6 months | 1200 mg/day, po | Beneficial |
| of NAC toward the formation of | | | | |
| lipophilic-DNA adducts, which can | | | | |
| modulate certain cancer-associated | | | | |
| biomarkers. VanShooten 2002[35] | | | | |
| Bronchopulmonary displasia. | 22 | 24 hours | 5% by tracheal | Adverse |
| Intratracheal NAC administration | | | administration | |
| neither improved clinical condition nor | | | | |
| hastened recovery in premature infants | | | | |
| with chronic lung disease. | | | | |
| Importantly, NAC administration in | | | | |
| this form led to increased total airway | | | | |
| resistance and cyanotic spells. Bibi, | | | | |
| 1992[203] | | | | |
| Bronchopulmonary dysplasia (BPD) | 391 | 6 Days | 16-32 mg/kg/d | not |
| NAC did not prevent BPD or death in | | | | signficant |
| infants with extremely low birthweight. | | | | |
| Ahola, 2003. [279] | | | | |
| Diaphragm dysfunction. | 4 | 3 weeks | 150 mg/kg, IV | Beneficial |
| Preadministration of IV NAC attenuates | | | | |
| the development of diaphragm fatigue in | | | | |
| normal subjects breathing against high | | | | |
| inspiratory loads. (Relevant to treatment | | | | |
| of patients with ventilatory failure | | | | |
| secondary to respiratory muscle fatigue.) | | | | |
| Travaline, 1997[37] | | | | |
| Mucociliary clearance rates. NAC has | 161 | 60 days | .6 g oral daily | Beneficial |
| a 35% improvement in mucusalary | | | | |
| clearance in subjects classified as slow | | | | |
| clearers and protects against lung | | | | |
| agressors. Todisco, 1985[38] | | | | |
| Elective pulmonary surgery. No | 38 | 5 days | 600 mg/day, po | not |
| benefit was demonstrated either for | | | | significant |
| postoperative pulmonary function or in | | | | |
| the frequency of atelectasis. Jepsen, | | | | |
| 1989[280] | | | | |
| Elective upper laparotomy. In patients | 131 | 6 days | 1200 mg pre- | not |
| subjected to elective upper laparotomy | | | surgery, po; | significant |
| given NAC prophylactically, no | | | 600 mg/day | |
| significant differences with respect to | | | postsurgery, po | |
| postoperative pulmonary function or in | | | | |
| the frequency of atelectasis were | | | | |
| detected between the NAC and placebo | | | | |
| groups. Jepsen, 1989[280] | | | | |
| ARDS. Parenteral NAC treatment | 16 | 3 days | 190 mg/kg/day, | Significant |
| started within 8 h of diagnosis increases | | | IV | (? Clin rel) |
| the intracellular GSH in the | | | | |
| granulocytes of ARDS patients without | | | | |
| decreasing spontaneous oxidant | | | | |
| production by these cells. Laurent, | | | | |
| 1996[215] | | | | |
| ARDS. No improvement could be | 66 | 6 days | 150 mg/kg bolus; | not |
| demonstrated in the PaO₂/FiO₂ ratio in | | | then 20 mg/kg/hr, | significant |
| the study group as compared with the | | | IV | |
| control group on any day. Findings | | | | |
| indicate that NAC acts as an | | | | |
| anticoagulant and perhaps decreases | | | | |
| pulmonary fibrin uptake during ARDS. | | | | |
| Jepsen, 1992[223] | | | | |
| ALI. Mechanical ventilatory support. | 61 | 3 days | 40 mg/kg/day, | Beneficial |
| IV NAC treatment improved systemic | | | IV | |
| oxygenation and reduced the need for | | | | |
| ventilatory support in patients | | | | |
| presenting with mild-to-moderate acute | | | | |
| lung injury. Development of ARDS and | | | | |
| mortality were not significantly reduced. | | | | |
| Suter, 1994[211] | | | | |
| ALI. Pretreatment with NAC prevents | 18 | 12 hours | 72 mg/kg bolus, | Beneficial |
| lung injury by diminishing elastase | | | 72 mg/kg over 12 | |
| activity in ALI The release of | | | hours, IV | |
| mediators, especially myeloperoxidase, | | | | |
| is not affected. De Backer, 1996[214] | | | | |
| ALI. NAC and OTZ both repleted RBC | 48 | 10 days | NAC, 70 mg/kg | Beneficial |
| GSH. The number of days of acute lung | | | q 8 hr, IV; | |
| injury was decreased and there was also | | | OTZ, 63 mg/kg | |
| a significant increase in cardiac index in | | | q 8 hr, IV | |
| both treatment groups. There was no | | | | |
| difference in mortality among groups. | | | | |
| Bernard, 1997[222] | | | | |

TABLE 1g
NAC use in septic shock and infectious disease

| NAC use in septic shock and infectious | | Study | | Beneficial |
|---|---|---|---|---|
| disease | n | length | Dosage | effects |
| Septic shock. NAC provided a transient | 58 | 2 hrs | 150 mg/kg for 15 | Beneficial |
| improvement in tissue oxygenation in | | | min, then 12.5 | |
| about half of the septic shock patients, as | | | mg/hr over 90 min, | |
| indicated by the increase in V02 and | | | IV | |
| gastric intramucosal pH and decrease in | | | | |
| veno-arterial PC⁰². The NAC responders | | | | |
| had a higher survival rate. Spies, | | | | |
| 1994[42] | | | | |
| Septic shock. Short-term IV infusion of | 22 | 24 hours | 150 mg/kg bolus; | Beneficial |
| NAC was well-tolerated, improved | | | then 50 mg/kg | |
| respiratory function and shortened ICU | | | over 4 hr, IV | |
| stay in survivors. Production of IL-8, a | | | | |
| potential mediator of septic lung injury, | | | | |
| was attenuated. Spapen, 1998[43] | | | | |
| Septic shock. After NAC treatment, | 60 | 2 hours | 150 mg/kg over 15 | Beneficial |
| hepatosplanchnic flow and function | | | min before 12.5 | |
| improved. The increase of liver blood | | | mg/kg/hr for 90 | |
| flow index was not caused by | | | min | |
| redistribution to the hepatosplanchnic | | | | |
| area, but by an increase of cardiac index. | | | | |
| Rank, 2000[44] | | | | |
| Cardiac performance in septic shock. | 20 | 48 hours | 150 mg/kg over 15 | Adverse |
| Early administration of NAC (within the | | | min; 50 mg/kg over | |
| first 2 hours of hemodynamic | | | next 4 hrs; 100 | |
| stabilization) resulted in a progressive | | | mg/kg over next 24 | |
| decrease in MAP, cardiac index, and left | | | hrs | |
| ventricular stroke work index relative | | | | |
| both to subject time-zero measurement | | | | |
| and to values for the placebo group (p < | | | | |
| .01, repeated-measures analysis of | | | | |
| variance). Peake, 1996[281] | | | | |
| Influenza. Long term treatment with oral | 262 | 6 months | 1200 mg/day, po | Beneficial |
| NAC during the cold-season appears to | | | | |
| significantly attenuate the frequency and | | | | |
| severity of influenza or influenza-like | | | | |
| episodes in elderly subjects and/or | | | | |
| patients suffering from chronic non- | | | | |
| respiratory diseases. De Flora, 1997[46] | | | | |
| HIV. In subjects with CD4 counts > | 45 | 4 months | 800 mg/day, po | Beneficial |
| 200/ul, NAC treatment resulted in | | | | |
| normalization of plasma cysteine levels | | | | |
| and a lower rate of decline in the CD4 | | | | |
| count. Akerlund, 1996[47] | | | | |
| HIV. Placebo-controlled study | 81 | 8 weeks | 3,200-8,000 mg/day, | Beneficial |
| established the long-term safety of NAC | | | po | |
| administration and demonstrated that | | | | |
| NAC treatment replenishes whole blood | | | | |
| GSH and T cell GSH in HIV infected | | | | |
| individuals. Viral load was not affected. | | | | |
| Replenishment was associated with | | | | |
| increased survival in the open-label | | | | |
| portion of the study. De Rosa, 2000 and | | | | |
| Herzenberg, 1997[48, 49] | | | | |
| HIV. NAC treatment but resulted in a | 69 | 7 months | 0.6-3.6 g depending | Beneficial |
| marked improvement in immune | | | on GSH levels, po | |
| function. It did not consistently alter | | | | |
| viral load. Subjects with and without | | | | |
| antiretroviral therapy (n = 40, n = 29, | | | | |
| respectively) were included. | | | | |
| Breitkreutz, 2000[50] (Also reviewed in | | | | |
| Droge 2000[282]) | | | | |
| HIV. A significant increase in whole | 37 | 4 weeks | OTC, 1500-3000 | Beneficial |
| blood GSH was seen after oral | | | mg/day, po | |
| administration of L-2-oxothiazolidine-4- | | | | |
| carboxylic acid (OTC), a cysteine pro- | | | | |
| drug, in the 1,500 mg and the 3,000 mg | | | | |
| dose groups. Barditch-Crovo, | | | | |
| 1998[283] | | | | |
| HIV. Individuals at the beginning of | 20 | 180 days | 600 mg/day | Beneficial |
| antiretroviral therapy were supplemented | | | | |
| with NAC or placebo. In NAC-treated | | | | |
| patients, hematocrit remained stable and | | | | |
| an increase in CD4 cell count took place | | | | |
| earlier than that in the control group. | | | | |
| Spada, 2002[227] | | | | |
| HIV. In subjects with CD4 counts < | 50 | 10 weeks | 800 mg/day po | Not |
| 200/ul, NAC did not replenish either | | | | significant |
| cysteine or GSH levels in plasma. NAC | | | | |
| did not significantly decrease the risk of | | | | |
| adverse reactions to trimethoprim- | | | | |
| sulphamethoxazole (TMP-SMX) in this | | | | |
| study. Akerlund, 1997[284] | | | | |
| Malaria. In severe encephalopathy | 30 | 20 hours | 300 mg/kg, IV | Beneficial |
| secondary to quinine-treated malaria, | | | | |
| serum lactate levels normalized twice as | | | | |
| quickly after NAC as after placebo. The | | | | |
| NAC-treated patients could be switched | | | | |
| from intravenous to oral therapy earlier | | | | |
| than individuals who received placebo, | | | | |
| but the difference was not significant. | | | | |
| Watt, 2002[51] | | | | |

TABLE 1h
NAC use in multiorgan failure

| | | Study | | Beneficial |
|---|---|---|---|---|
| NAC use in multiorgan failure | n | length | Dosage | effects |
| Hemodynamic effects. NAC treatment | 10 | 45 min | 150 mg/kd | Significant |
| increased cardiovascular index and | | | | (? Clin rel) |
| vasodilation but did not affect oxygen | | | | |
| extraction or lactate concentration. Agusti, | | | | |
| 1997. [45] | | | | |
| Intensive care. There was no statistically | 50 | 3 to 5 days | Intravenous | not |
| significant difference between the two groups | | | admin of 150 | significant |
| regarding outcome as indicated by mortality | | | mg/kg followed | |
| and the required days of inotropic support, | | | by 12/mg/kg | |
| mechanical ventilation, and intensive care. | | | | |
| Molnar, 1998[285] | | | | |

TABLE 1i
NAC use in neurologic and musculoskeletal pathophysiology

| NAC use in neurologic and musculoskeletal | | Study | | Beneficial |
|---|---|---|---|---|
| pathophysiology | n | length | Dosage | effects |
| Alzheimer's Disease. Comparison of interval | 47 | 24 weeks | 50 mg/kg/day | Beneficial |
| change favored NAC treatment as measured by | | | | |
| MMSE, the ADL (Activity of Daily Life) scal | | | | |
| and other psychometric tests. Adair, 2001[60] | | | | |
| Amyotrophic Lateral Sclerosis. Treatment | 110 | 1 year | 50 mg/kg daily | not |
| of subjects with ALS did not result in a major | | | | significant |
| increase in 12 month survival or reduction in | | | | |
| disease progression. Louwerse, 1995[242] | | | | |
| Exercise. NAC attenuated the decrease in | 8 | | 125 mg/kg/hr | Beneficial |
| reduced GSH and the increase in oxidized | | | for 15 min, then | |
| GSH resulting from intense, intermittent | | | 25 mg/kg/hr | |
| exercise. Time to fatigue was unchanged. | | | | |
| Medved, 2003[58] | | | | |
| Exercise. This is the first demonstration that | | | 125 mg/kg/hr | Beneficial |
| NAC infusion during prolonged submaximal | | | for 15 min, | |
| exercise sunstantially enhanced performance in | | | then 25 mg/kg/20 | |
| well trained individuals. Medved, 2004. [286] | | | min | |
| Exercise. In frail geriatric patients responding | 44 | 6 weeks | 1800 mg daily | Beneficial |
| to physical exercise, NAC enhanced knee | | | | |
| extensor strength, increased the sum or all | | | | |
| strength parameters and decreased plasma | | | | |
| TNF-□. Hauer, 2003[59] | | | | |

TABLE 1j
NAC use in otolaryngolic, opthamalogic and dental pathophysiology

| NAC use in otolaryngolic, opthamalogic and | | Study | | Beneficial |
|---|---|---|---|---|
| dental pathophysiology | n | length | Dosage | effects |
| Otitis Media. Instillation of NAC into one ear | 75 | 39 months | 0.05 ml Mucomyst | Beneficial |
| at the time of bilateral insertion of ventilation | | | solution at 1, 3 | |
| tubes (VTs) and on days 3 and 7 afterwards | | | and 7 days | |
| decreased recurrence of otitis media with | | | | |
| effusion, decreased re-insertion of VTs, and | | | | |
| increased time until VT extrusion. The | | | | |
| number of episodes of ear problems and visits | | | | |
| to the ENT clinic were decreased. Ovesen, | | | | |
| 2000[53] | | | | |
| Sjogren's syndrome. NAC treatment | 26 | 6 weeks | 200 mg daily | Beneficial |
| improved ocular soreness (p = 0.004), ocular | | | | |
| irritability (p = 0.006), halitosis (p = 0.033) | | | | |
| and daytime thirst (p = 0.033). Walters, | | | | |
| 1986[56] | | | | |
| Chronic blepharitis. NAC improves tear film | 40 | 4 months | 300 mg/day, po | Beneficial |
| quality as detected by fluorescein break-up | | | | |
| time (FBUT) and mucous fern pattern. Yalcin, | | | | |
| 2002[57] | | | | |
| Prevention of plaque formation. Comparison | 28 | 2 weeks | 10% aqueous | Beneficial |
| of plaque indices for control and test periods | | | solution of | |
| showed a statistically significant (p < .001) | | | NAC buffered | |
| 25.56% reduction on plaque attributable to | | | to pH 6.5 with | |
| NAC. Bowles, 1985[61] | | | flavoring agents | |
| | | | added | |

TABLE 1k
NAC use in dermatologic pathophysiology

| NAC use in dermatologic | | Study | | Beneficial |
|---|---|---|---|---|
| pathophysiology | n | length | Dosage | effects |
| Progressive Systemic Sclerosis. | 22 | I year | 1000- | not |
| Several simple measures of | | | 2000 mg | significant |
| function and skin distensibility | | | daily | |
| were tested. No significant | | | | |
| differences between the NAC | | | | |
| and placebo groups were observed | | | | |
| over the year of the study. | | | | |
| Furst, 1979[287] | | | | |

TABLE 2
NAC treatment: reports of observational studies

| | | | | Clinical |
|---|---|---|---|---|
| | | | | findings |
| Classi- | | Clinical | GSH | plus GSH |
| fication | Disease | findings | measures | measures |
| Hepatic | Acetaminophen | [2, 71, | | [128, 133, |
| Function | Toxicity | 110, | | 134, 136, |
| | | 122, 124, | | 159, 289] |
| | | 126, 131, | | |
| | | 135, 137, | | |
| | | 138, 140, | | |
| | | 141, 146- | | |
| | | 148, 152, | | |
| | | 153, 288] | | |
| | Alcohol | [290] | [168] | [291, 292] |
| | Hepatitis | [161, 293, | | [295- |
| | | 294] | | 299] |
| Renal | Chronic | [300] | | [301] |
| Function | Kidney | | | |
| | Failure | | | |
| | Dialysis | [302] | | |
| | Nephrotoxicity | [3, 190] | | |
| | □-Amanintin | [160] | | |
| Cardio- | Angina | | | |
| vascular | | | | |
| | Arteriosclerosis/ | [303- | [308] | [307, 309] |
| | Cardiac Risk | 307] | | |
| | Myocardial | | | [310] |
| | Infarction | | | |
| | Cardiomyopathy | | | [311] |
| Endocrine | Diabetes | [312- | | [106, 315- |
| | | 314] | | 323] |
| Pulmonary | Reviews | [324- | [327, | [329] |
| | | 326] | 328] | |
| | Bronchopulmonary | [330, | | |
| | | 331] | | |
| | ARDS | | [332] | [333, 334] |
| | Fibrosing | | | [335, 336] |
| | Aveolitis | | | |
| | Chronic Asthma | [23, | | |
| | | 337] | | |
| | Chronic | [338- | | |
| | Bronchitis/ | 340] | | |
| | COPD | | | |
| | Cystic | [341] | | [178, 342- |
| | Fibrosis | | | 346] |
| | Pulmonary | | [347, | [349- |
| | Fibrosis | | 348] | 352] |
| | Smoking | [35] | | [36, 353- |
| | | | | 356] |
| | Lung Cancer | [357] | | |
| Critical | Intensive | | | [358- |
| Care | Care | | | 360] |
| | Sepsis/ | [42, | | [43, 364, |
| | Septic Shock | 361- | | 365] |
| | | 363] | | |
| | Malnutrition | | [173, | [54, 170, |
| | | | 366] | 171] |
| Infection | HIV | [367- | [370- | [379- |
| | | 369] | 378] | 388] |
| | Helicobacter | | [389] | [390] |
| | pylori | | | |
| | Influenza | | [391] | |
| | Malaria | | | |
| | Epilepsy | | [392, | [394, 395] |
| | | | 393] | |
| Gastro- | Inflammatory | | [396] | [397- |
| intestinal | Bowel | | | 399] |
| | Disease | | | |
| | Barrett's | | | [400] |
| | Esophagus | | | |
| | Liver Disease | [401- | [143] | [406, 407] |
| | | 405] | | |
| | Liver | | | [81] |
| | transplantation | | | |
| | Colon Cancer | | | [408- |
| | | | | 411] |
| Optic | Blepharitis | | | |
| | Cataract | [412- | | |
| | | 414] | | |
| | Eale's Disease | | | [415] |
| Skin | Psoriasis | [416] | | [417] |
| | Photodermatosis | | | [418] |
| Immune | Rheumatoid | | | [419- |
| System | Arthritis | | | 421] |
| | Comm. Variable | | | [422] |
| | Immunodeficiency | | | |
| Urogenital | Prostate | [423- | | [426, |
| | | 425] | | 427] |
| | Urinary | | | |
| Aging | | [428- | | [431- |
| | | 430] | | 436] |
| Toxic | Arsenic | [437] | | |
| Agents | Poisoning | | | |
| | Other | [438] | [439- | |
| | Chemicals and | | 441] | |
| | Medications | | | |
| Perinatal | Preeclampsia | [442, 443] | | |
| | Neonates | | | [444- |
| | | | | 446] |
| Metabolism | Phenylketonuria | | | [447] |
| Misc. | | [448- | | [241, 452- |
| Reviews | | 451] | | 454] |

Cancer Treatment Review

1. A method of treatment to prevent development of glutathione deficiency as a consequence of disease, a treatment or a condition comprising administering to a subject at risk of glutathione deficiency as a consequence of disease, or a treatment, or a condition, a therapeutic amount of N-acetylcysteine or a pharmaceutically acceptable salt or derivative thereof sufficient to increase intracellular glutathione levels or decrease oxidative stress and measuring and monitoring the level of glutathone in blood in patients as needed.

2. A method of treatment to restore glutathione levels comprising administering to subjects in need of glutathione level restoration as determined by measurement or by physician a therapeutic amount of N-acetylcysteine or a pharmaceutically acceptable salt or derivative thereof sufficient to increase intracellular glutathione levels or decrease oxidative stress and monitoring restoration by measuring the level of glutathione in blood as needed.

3. A method of treatment to decrease oxidized glutathione levels elevated as a consequence of disease, a treatment or a condition, comprising administering to a subject suffering from oxidative stress a therapeutic amount of N-acetylcysteine or a pharmaceutically acceptable salt or derivative thereof sufficient to decrease oxidized glutathione levels elevated as a consequence of disease, a treatment, or a condition and monitoring the level of oxidized glutathione in blood as needed.
